(19) United States
(12) Patent Application Publication
(10) Pub. No.: US 2002/0022026 A1
(43) Pub. Date: Feb. 21, 2002

(54) Genes associated with mechanical stress, expression products therefrom, and uses thereof

(76) Inventors: Paz Einat, Ness-Ziona (IL); Orit Segev, Rohovot (IL); Rami Skaliter, Ness-Ziona (IL); Elena Feinstein, Rehovot (IL); Alexander Faerman, Bnei Aiish (IL)

Correspondence Address: Susan Lehnhardt, FORMMER LAWRENCE & HAUG LLP, 745 Fifth Avenue, New York, NY, 10151, US

(21) Appl. No.: 09/729,485

(22) Filed: Dec. 4, 2000

Related U.S. Application Data
(63) Continuation-in-part of application No. 09/632,862, filed on Aug. 4, 2000.
(60) Provisional application No. 60/207,821, filed on May 30, 2000.
(60) Provisional application No. 60/084,944, filed on May 11, 1998.
(60) Provisional application No. 60/085,673, filed on May 15, 1998.

(51) Int. Cl. A61K 39/395; C12Q 1/68; C07H 21/04; C12N 9/00; C12P 21/02

(52) U.S. Cl. 424/131.1; 435/6; 435/69.1; 536/23.2; 435/183; 435/320.1

(57) ABSTRACT

The disclosure relates to mechanical stress induced genes, such as those from human and from mice, probes therefor, tests to identify such genes, expression products of such genes, uses for such genes and expression products, e.g., in diagnosis (for instance risk determination), treatment, prevention, or control, of osteoporosis or factors or processes which lead to osteoporosis; and, to diagnostic, treatment, prevention, or control methods or processes, as well as compositions therefor and methods or processes for making and using such compositions.

### CROSS-REFERENCE TO RELATED APPLICATION

This application claims the priority of U.S. Provisional application Serial No. 60/207,821, filed May 30, 2000 and U.S. Ser. No. 09/632, 862, filed Aug. 4, 2000.

Reference is also made to U.S. Provisional application Serial No. 60/084,944, filed May 11, 1998, and the full U.S. utility application, Ser. No. 09/309,862, filed May 11, 1999, naming as inventors Paz Einat, Rami Skaliter, Oma Mor and Sylvie Luria and assigned to the assignee of the present application (Kohn & Associates Attorney Docket No. 0168.00060), and claiming priority from U.S. Provisional application Serial No. 60/084,944 (herein “the May 14, 1999 Einat et al. full U.S. utility application”), and U.S. Ser. No. 09/312,216, filed, May 14, 1999 and U.S. Provisional application Serial No. 60/085,673, filed May 15, 1998. U.S. Provisional application Serial No. 60/085,673, filed May 15, 1998, U.S. Provisional application Serial No. 60/207,821, filed May 30, 2000, U.S. Ser. No. 09/312,216, filed, May 14, 1999, U.S. Provisional application Serial No. 60/084,944, and the May 11, 1999 Einat et al. full U.S. utility application, as well as each document or reference cited in these applications, are hereby expressly incorporated herein by reference. Documents or references are also cited in the following text, either in a Reference List before the claims, or in the text itself; and, each of these documents or references (“herein-cited documents or references”), as well as each document or reference cited in each of the herein-cited documents or references, are hereby expressly incorporated herein by reference. It is explicitly stated that the inventive entity of the May 11, 1999 Einat et al. full U.S. utility application is not another or others as to the inventive entity of this application; and, that the inventive entity of the present application is not another or others as to the inventive entity of the May 11, 1999 Einat et al. full U.S. utility application.

### STATEMENT REGARDING FEDERALLY SPONSORED RESEARCH

Not Applicable.

### FIELD OF THE INVENTION

This invention relates to mechanical stress induced genes, probes therefor, tests to identify such genes, expression products of such genes, uses for such genes and expression products, e.g., in diagnosis (for instance risk determination), treatment, prevention, or control, of osteoporosis or factors or processes which lead to osteoporosis, osteopenia, osteopetrosis, osteosclerosis, osteoarthritis, periodontosis and bone fractures; and, to diagnosis, treatment, prevention, or control methods or processes, as well as compositions therefor and methods or processes for making and using such compositions.

### BACKGROUND OF THE INVENTION

Bone is composed of a collagen-rich organic matrix impregnated with mineral, largely calcium and phosphate. Two major forms of bone exist, compact cortical bone forms the external envelopes of the skeleton and trabecular or medullary bone forms plates that traverse the internal cavities of the skeleton. The responses of these two forms to metabolic influences and their susceptibility to fracture differ.

Bone undergoes continuous remodeling (turnover, renewal) throughout life. Mechanical and electrical forces, hormones and local regulatory factors influence remodeling. Bone is renewed by two opposing activities that are coupled in time and space (Parfitt 1979). These activities—resorption and formation—are contained within a temporary anatomic structure known as a bone-remodeling unit (Parfitt 1981). Within a given bone-remodeling unit, old bone is resorbed by osteoclasts. The resorbed cavity created by the osteoclasts is subsequently filled with new bone by osteoblasts, which synthesize the organic matrix of bone.

Peak bone mass is mainly genetically determined, though dietary factors and physical activity can have positive effects. Peak bone mass is attained at the point when skeletal growth ceases, after which time bone loss starts.

In contrast to the positive balance that occurs during growth, in osteoporosis, the resorbed cavity is not completely refilled by bone (Parfitt 1988). Osteoporosis, or porous bone, is a progressive and chronic disease characterized by low bone mass and structural deterioration of bone tissue, leading to bone fragility and an increased susceptibility to fractures of the hip, spine, and wrist (diminishing bone strength).

Bone loss occurs without symptoms. The Consensus Development Conference (Am. J. Med. 94:646-50, 1993) defined osteoporosis as “a systemic skeletal disease characterized by low bone mass and microarchitectural deterioration of bone tissue, with a consequent increase in bone fragility and susceptibility to fracture.”

Common types of osteoporosis include postmenopausal osteoporosis; and senile osteoporosis, which generally occurs in later life, e.g., 70+ years; see, e.g., U.S. Pat. No. 5,691,153. Osteoporosis is estimated to affect more than 25 million people in the United States (Rosen 1997); and, at least one estimate asserts that osteoporosis affects 1 in 3 women (Keen et al. 1997). Moreover, life expectancy has increased, and in the western world, 17% of women are now over 50 years of age; and, a woman can expect to live one third (⅓) of her life after menopause. Thus, some estimate that 1 out of every 2 women and 1 out of 5 men will eventually develop osteoporosis; and, that 75 million people in the US, Japan and Europe have osteoporosis. The World Summit of Osteoporosis Societies estimates that more than 200 million people worldwide are afflicted with the disease. The actual incidence of the disease is difficult to estimate since the condition is often asymptomatic until a bone fracture occurs. It is believed that there are over 1.5 million osteoporosis-associated bone fractures per year in the U.S. of which 300,000 are hip fractures that usually require hospitalization and surgery and may result in lengthy or permanent disability or even death. (See Spangler et al. “The Genetic Component of Osteoporosis Mini-review”; http://www.csa.com.osteointro.html).

Osteoporosis is also a major health problem in virtually all societies (Eisman 1996; Wark 1996; U.S. Pat. No. 5,834,200). There is a 20-30% mortality rate related to hip fractures in elderly women (U.S. Pat. No. 5,691,153); and, such a patient with a hip fracture has a 10-15% greater chance of dying than others of the same age. Further, although men suffer fewer hip injuries than women, men are 25% more likely than women to die within one year of the injury. See Sprangler et al., supra. Also, about 20% of the patients who were living independently before a hip fracture still remain confined in a long-term health care facility one year later. The treatment of osteoporosis and related fractures can cost over $10 billion annually.

Treatment for osteoporosis helps stop further bone loss and fractures. Common therapeutics include HRT (hormone replacement therapy), bisphosphonates, e.g., alendronate (Fosamax), as well as, estrogen and estrogen receptor modulators, progestin, calcitonin, and vitamin D. While there may be numerous factors that determine whether any particular person will develop osteoporosis, a step towards prevention, control or treatment of osteoporosis is determining whether one is at risk for osteoporosis. Genetic factors also play an important role in the pathogenesis of osteoporosis (Ralston 1997; see also Keen et al. 1997; Eisman 1996; Rosen 1997; Cole 1998, Johnston et al. 1995; Gong et al. 1996; Wasnich 1996 inter alia). Some attribute 50-60% of total bone variation (Bone Mineral Density; BMD), depending upon the bone area, to genetic effects (Livshits et al. 1996). However, up to 85%-90% of the variance in bone mineral density may be genetically determined.

Studies have shown from family histories, twin studies, and racial factors, that there may be a predisposition for osteoporosis (see, e.g., Jouanny et al. 1995; Garnero et al. 1996; Cummings 1996; Lonzer et al. 1996). Several candidate genes may be involved in this, most probably multigenic, process.

Cytokines are powerful regulators of bone resorption and formation under control of estrogen/testosterone, parathyroid hormone and 1,25(OH)2D3. Some cytokines primarily enhance osteoclastic bone resorption e.g. IL-1, TNF (Tumor Necrosis Factor) and IL-6 (Interleukin-6), while others primarily stimulate bone formation e.g. TGF-β (Transforming Growth Factor), IGF (Insulin-like Growth Factor) and PDGF (Platelet Derived Growth Factor).

There is need for clinical and epidemiological research for the prevention and treatment of osteoporosis for gaining deeper knowledge of factors controlling bone cell activity and regulation of bone mineral and matrix formation and remodeling.

Bone develops via a number of processes. Mesenchymal cells can differentiate directly into bone, as occurs in the flat bones of the craniofacial skeleton; this process is termed intramembranous ossification. Alternatively, cartilage may provide a template for bone morphogenesis, as occurs in the majority of human bones. The cartilage template is replaced by bone in a process known as endochondral ossification (see Reddi, 1981 #2). Bone is also continuously modeled during growth and development and remodeled throughout the life of the organism in response to physical and chemical signals. Development and maintenance of cartilage and bone tissue during embryogenesis and throughout the life-time of vertebrates is very complex. It is widely accepted that a multitude of factors, from systemic hormones to local regulatory factors such as the members of the TGF-β superfamily, cytokines and prostaglandins, act in concert to regulate the continuous processes of bone formation and bone resorption. Disturbance of the balance between osteoblastic bone deposition and osteoclastic bone resorption is responsible for many skeletal diseases.

Diseases of bone loss are a major public health problem especially for women in all Western communities. The most common cause of osteopenia is osteoporosis; other causes include osteomalacia and bone disease related to hyperparathyroidism. Osteopenia has been defined as the appearance of decreased bone mineral content on radiography, but the term more appropriately refers to a phase in the continuum from decreased bone mass to fractures and infirmity. It is estimated that 30 million Americans are at risk for osteoporosis, the most common among these diseases, and there are probably 100 million people similarly at risk worldwide (Melton, 1995 #3). These numbers are growing as the elderly population increases. Despite recent successes with drugs that inhibit bone resorption, there is a clear need for specific anabolic agents that will considerably increase bone formation in people who have already suffered substantial bone loss. There are no such drugs currently approved.

Mechanical stimulation induces new bone formation in vivo and increases osteoblastic differentiation and metabolic activity in culture. Mechanotransduction in bone tissue involves several steps: 1) mechanochemical transduction of the signal, 2) cell-to-cell signaling, and 3) increased number and activity of osteoblasts. Cell-to-cell signaling after mechanical stimulus involves prostaglandins, especially those produced by COX-2 and nitric oxide. Prostaglandins induce new bone formation by promoting both proliferation and differentiation of osteoprogenitor cells. In a search for agents that enhance osteoblast proliferation/differentiation and bone formation, mechanical force has been employed in the present invention as an inducer of osteogenesis and our proprietary gene discovery methodology carried out to detect genes that are specifically expressed in very early osteo/chondro-progenitor cells.

### OBJECTS AND SUMMARY OF THE INVENTION

The present invention provides mechanical stress induced genes, expression products of such genes, uses for such genes and expression products for treatment, prevention, control, of osteoporosis or factors or processes which lead to osteoporosis, osteopenia, osteopetrosis, osteosclerosis, osteoarthritis, periodontosis and bone fracture. The invention further provides diagnostic, treatment, prevention, control methods or processes as well as compositions.

The invention additionally provides an isolated nucleic acid molecule encoding the protein 608 or a functional portion thereof or a polypeptide, which is at least substantially homologous or identical thereto. The invention comprehends an isolated nucleic acid molecule encoding human protein 608 (or “OCP”) or a functional portion thereof.

The invention further encompasses methods for preventing, treating or controlling osteoporosis or low bone density or other factors causing or contributing to osteopenia, osteopetrosis, osteosclerosis, osteoarthritis, periodontosis or symptoms thereof, or other conditions involving mechanical stress or a lack thereof, comprising administering an inventive polypeptide or portion thereof; and accordingly, the invention comprehends uses of polypeptides in preparing a medicament or therapy for such prevention, treatment or control.

The invention also comprehends a method for preventing, treating or controlling osteoporosis or low bone density or other factors causing or contributing to osteoporosis or symptoms thereof or other conditions involving mechanical stress or a lack thereof, comprising administering a composition comprising a gene or functional portion thereof, an antibody or portion thereof elicited by such an expression product or portion thereof; and, the invention thus further comprehends uses of such genes, expression products, antibodies, portions thereof, in the preparation of a medicament or therapy for such control, prevention or treatment.

These and other embodiments are disclosed or are obvious from and encompassed by, the Detailed Description which follows the Brief Description of the Figures below.

### BRIEF DESCRIPTION OF THE FIGURES

The following Detailed Description, given by way of example, but not intended to limit the invention to specific embodiments described, may be understood in conjunction with the accompanying Figures, in which:

FIG. 1 describes the cDNA sequence for full rat 608 gene (SEQ ID NO:1).

FIG. 2 describes PcDNA3.1-608 construct.

FIG. 3 describes OCP rat amino acid sequence (SEQ ID NO:2).

FIG. 4 describes TNT (transcription - translation) assays.

FIG. 5 describes the structure of Bac 23-261L4.

FIG. 6 describes the structure of Bac 23-241H7. (Note that the region upstream nt. no. 453 was not sequenced and probably carries the rat OCP 5'UTR.

FIG. 7 describes sequence analysis of m608p-Lexicon clone (SEQ ID NO:3) - Partial re-sequence. (1) Re-sequenced regions are underlined. These regions are different at some points from the sequence sent by Lexicon; (2) Putative exons are in Bold lettering; (3) ATG-First ATG of coding region (in Italics).

FIG. 8 describes mouse OCP exon & intron map.

FIG. 9 describes OCP map of exon-intron borders.

FIG. 10 describes sequence alignment between genomic human OCP (SEQ ID NO:4) and cDNA rat (SEQ ID NO:5) - 2 exons.

FIG. 11 describes human OCP exon and intron list.

FIG. 12 describes OCP human cDNA sequence (SEQ ID NO:6).

FIG. 13 shows the % identity between human, mouse and rat cDNA and protein.

FIG. 14 shows the alignment of rat, mouse, and human OCP cDNA coding regions (rat_cDNA: SEQ ID NO:7, human_{—}5+3 corrected: SEQ ID NO:8, and mus_cDNA_{—}5: SEQ ID NO:9).

FIG. 15 shows the alignment of rat, human and mouse OCP proteins (rat: SEQ ID NO:10, human_{—}5+3_corrected: SEQ ID NO:11, and mouse_{—}5_corrected: SEQ ID NO:12).

FIG. 16 shows the alignment of rat and human OCP proteins (rat: SEQ ID NO:13 and human_{—}5+3_corrected: SEQ ID NO:14).

FIG. 17 describes OCP partial mouse protein (236 aa) (SEQ ID NO:15).

FIG. 18 describes OCP human protein (2587 aa) (SEQ ID NO:16).

FIG. 19 describes the OCP protein structure from the OCP gene.

FIG. 20 shows a list of expression patterns of OCP in primary cells and various other cell lines. Northern blot of poly A+RNA from rat primary calvaria cells and MC3T3 cells is shown. As can be seen, the main 8.9Kb transcript is present only in calvaria cells. RT-PCR assays with specific OCP primers were performed on total RNA from various lines as indicated on the right side of the figure. In all assays similar amounts of GapDH RT-PCR products were detected in all RNA samples. In addition, no GapDH products were detected in any RNA samples, when RT was omitted. (−) represents no expression of OCP, while (+) represents expression. When (−+) are indicated, the expression of OCP is induced only upon specific conditions.

FIG. 21 shows the effects of mechanical stress on MC3T3 pre-osteoblastic cells. RT-PCR for OCP, Cbfal, Osteopontin (OPN) and GAPDH transcripts as indicated. The results shown are representative of three experiments using total cellular RNA from MC3T3 cells that did not undergo mechanical stress (1), and mechanical stimulated MC3T3 cells (2). The RT-PCR products that are marked, were visualized by staining with ethidium bromide.

FIG. 22 describes OCP (608) expression in early stages of osteoblast differentiation.

FIG. 23 shows that OCP is an early marker of endochondral ossification.

FIG. 24 shows that OCP is induced during osteoblastic differentiation of bone marrow stroma cells.

FIG. 25 describes in vivo regulation of OCP expression by various treatments. The results shown are representative of three experiments using total cellular RNA from treated two-months old mice. The different treatments are indicated. The RT-PCR products are marked. Control mice did not undergo any treatment. In each treatment group the left lane represents negative control without the addition of RT, the central lane represents the OCP RT-PCR product and the right lane represents the GapDH RT-PCR product.

FIG. 26 describes a low power microphotograph of the fractured bone one week after the operation. Note that well-developed woven bone and fibrocartilagenous callus formed at the fracture site. Bone marrow tissue was mainly destroyed by insertion of the wire used for the fracture immobilization. Marked areas are presented at higher magnification on the following figures.

FIG. 27 shows microphotographs of the central part of callus, brightfield (left) and darkfield (right). Cells expressing the OCP gene can be seen in fibrous part of callus. There is no hybridization signal from chondrocytes.

FIG. 28 shows microphotographs of the callus area marked by 2 in FIG. 26.

Brightfield (left) and darkfield (right). Cells expressing the OCP gene can be seen in highly vascularized subperiosteal area bordering the cartilagenous part of the callus.

FIG. 29 shows microphotographs of the highly vascularized endosteal tissue. This was developed in reaction to the wire insertion (area 3 on FIG. 26), brightfield (upper) and darkfield (lower). This tissue contains many cells expressing the OCP gene.

FIG. 30 shows high power microphotograph showing perivascular cells. The perivascular cells express the 608 gene within lacuna of woven bone arrowheads.

FIG. 31 shows high power microphotograph of periosteum covering the woven bone.

Multiple cells display expression of the 608 gene in periosteum. Arrowheads point to two 608 expressing cells within the woven bone.

FIG. 32 shows brigthfield (left and darkfield (right) microphotographs of section of fractured bone healed for 4 weeks. Multiple cells in periosteal tissue—area of active remodeling of the cancellous bone—covering the callus show hybridization signal.

FIG. 33 shows the boxed area of FIG. 32 presented at higher magnification. Several OCP expressing cells are concentrated in vascular tissue that fills the cavities resulting from osteoclast activity (marked by astrisks).

FIG. 34 shows induction of osteoblastic differentiation by transfected OCP.

FIG. 35 describes the transient transfections of OCP deletion constructs to calvaria cells. Two OCP deletion constructs (OCP-403, OCP-760) and OCP full length construct were transiently transfected to primary calvaria cells. ALP staining is presented. As can be seen all deletion constructs show increased osteoblastic colony no. and colony size compare with transient transfection of the control pCDNA vector.

FIG. 36 shows an increased osteoblasts differentiation in OCP-transfected ROS cells.

RT-PCR assays with OCP, Cbfal, ALP, BSP & GapDH specific primers as indicated above. The results shown are representative of two experiments using total cellular RNA from (1)- the stable OCP-expressed ROS cell line, and (2)- the control ROS cell line (stable transfection with pCDNA. The OCP RT-PCR product is 1020 bp, the Cbfal product is 289 bp, the ALP product is 226 bp, the BSP product is 1048 bp and the GapDH (control) product is 450 bp long. M—represents protein markers.

FIG. 37 shows an increased osteoblasts proliferation in OCP-transfected ROS cells.

FIG. 38 describes an induction of bone formation ex vivo.

FIG. 39 shows the structure of the Osteocalcin promoter - OCP gene.

FIG. 40 shows autoradiograms of the Southern blot analysis of placenta DNAs. “A” shows the results of Southern blot done on the DNAs from all embryos that developed. (Note that sample 10 is missing due to lack of embryo in the sample.) “F” is the injected fragment, served as positive control for the expected size. The arrow marks the expected fragment. “B” shows a section of the autoradiogram of “A” which was exposed to the sample for additional time. These autoradiograms show that both embryos 20 and 21 are transgenic. “F” is the injected fragment, served as positive control for the expected size. The arrow marks the expected fragment. “C” shows a repetition of the Southern blot on DNA from three selected embryos, i.e. embryos 11, 20 and 21. Embryos 20 and 21 are again detected as transgenic. Embryo 11, which gave an obscured signal on the longer exposure of “A” (not shown), is also detected as transgenic in “C.” “F” is a genomic DNA from a stable transgenic line produced later. The correct fragment is indicated by an arrow. The more intense fragment found below is a non-specific fragment occasionally observed with the SV40 probe.

FIG. 41 shows the exogenic OCP expression in transgenic embryos. RT-PCR for exogenic OCP transcripts was performed. The results are representative of three experiments using total cellular RNA from embryos tails. The RT-PCR products that are marked, were visualized by staining with ethidium bromide. GapDH primers were used to show that differences in OCP transcript abundance did not reflect variation in the efficiency of the RT reaction.

FIG. 42 describes the characterization of osteocalcin promoter of OCP transgenic embryos (E17 embryos). Calvaria, tibia & femur lengths were measured in μm. All the measurments include only the calcified regions that were stained by Alizarin Red.

FIG. 43 shows OC-OCP transgenic embryos long bones using Alizarin Red staining.

FIG. 44 describes Alizarin Red staining of calvaria bones from transgenic and control embryos. Higher calcification (represented by Alizarin Red staining) was detected when transgenic embryos calvaria bones were stained in comparison with their littermate. In addition, the calvaria bones of the transgenic embryos were measured and were found to be longer and wider.

FIG. 45 shows clone 14C10 structure compared to the Lexicon clone structure.

FIG. 46 shows the structure of the pMCSIEm608prm5.5 construct.

FIG. 47 shows the sequence of the mouse's OCP promoter region (proximal 5.5Kb fragment) (SEQ ID NO:17).

FIG. 48 shows the sequence of the 5′ end of clone p14C10 (SEQ ID NO:18).

FIG. 49 shows the proximal part of the regulatory region of the human and mouse OCP Gene.

FIG. 50 shows the sequences of the primer (SEQ ID NO:19) and the QB3 (CMF608) (SEQ ID NO:20).

### DETAILED DESCRIPTION OF THE INVENTION

The present invention is related to the discovery of a novel gene, CMF608 (renamed “OCP”.), that was found to be upregulated by mechanical stress on primary cavaria cells, and describe several functional features that identify it as the most specific early marker of osteo- or chondro-progenitor cells as well as an inducer of osteoblast proliferation and differentiation.

As used herein, the same gene of the invention may be referred to either as “608” or “OCP”. RNA refers to RNA isolated from cell cultures, cultured tissues or cells or tissues isolated from organisms which are stimulated, differentiated, exposed to a chemical compound, are infected with a pathogen or otherwise stimulated. As used herein, translation is defined as the synthesis of protein on an mRNA template.

As used herein, stimulation of translation, transcription, stability or transportation of unknown target mRNA or stimulating element, includes chemically, pathogenically, physically, or otherwise inducing or repressing an mRNA population from genes which can be derived from native tissues and/or cells under pathological and/or stress conditions. In other words, stimulating the expression of a gene's mRNA with a stress inducing element or “stressor” can include the application of an external cue, stimulus, or stimuli which stimulates or initiates translation of a mRNA stored as untranslated mRNA in the cells from the sample. The stressor may cause an increase in stability of certain mRNAs, or induce the transport of specific mRNAs from the nucleus to the cytoplasm. The stressor may also induce gene transcription. In addition to stimulating translation of mRNA from genes in native cells/tissues, stimulation can include induction and/or repression of genes under pathological and/or stress conditions. The present method utilizes a stimulus or stressor to identify unknown target genes which are regulated at the various possible levels by the stress inducing element or stressor.

More in particular, with respect to the herein mentioned nucleic acid molecules and polypeptides, e.g., the aforementioned nucleic acid molecules (rat 608 and human 608 genes) and polypeptides expressed from them, the invention further comprehends isolated and/or purified nucleic acid molecules and isolated and/or purified polypeptides having at least about 70%, preferably at least about 75% or about 77% identity or homology (“substantially homologous or identical”), advantageously at least about 80% or about 83%, such as at least about 85% or about 87% homology or identity (“significantly homologous or identical”), for instance at least about 90% or about 93% identity or homology (“highly homologous or identical”), more advantageously at least about 95%, e.g., at least about 97%, about 98%, about 99% or even about 100% identity or homology (“very highly homologous or identical” to “identical”; or from about 84-100% identity considered “highly conserved”). The invention also comprehends that these nucleic acid molecules and polypeptides can be used in the same fashion as the herein or aforementioned nucleic acid molecules and polypeptides.

Nucleotide sequence homology can be determined using the “Align” program of Myers and Miller, (CABIOS 4:11-17, 1988) and available at NCBI. Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence similarity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur and Lipman, 1983 PNAS USA 80:726), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence (see also alignment used in Figures).

RNA sequences within the scope of the invention can be derived from DNA sequences, by substituting thymidine (T) in the DNA sequence with uracil (U).

Additionally or alternatively, amino acid sequence similarity or identity or homology can be determined using the BlastP program (Altschul et al., Nucl. Acids Res. 25:3389-3402) and available at NCBI. The following references provide algorithms for comparing the relative identity or homology of amino acid residues of two proteins, and additionally or alternatively with respect to the foregoing, the teachings in these references can be used for determining percent homology or identity: Smith et al. Advances in Applied Mathematics 2:482-489 (1981); Smith et al. Nucl. Acids Res. 11:2205-2220 (1983); Feng et al. J. Molec. Evol., 25:351-360 (1987); Higgins et al. CABIOS, 5: 151-153 (1989); Thompson et al. Nucl. Acids Res. 22:4673-480 (1994); and, Devereux et al. Nucl. Acids Res., 12:387-395 (1984).

As to uses, the inventive genes and expression products as well as genes identified by the herein disclosed methods and expression products thereof (including “functional” variations of such expression products, and truncated portions of herein defined genes such as portions of herein defined genes which encode a functional portion of an expression product) are useful in treating, preventing or controlling or diagnosing mechanical stress conditions or absence or reduced mechanical stress conditions.

For instance, 608 expression appears to cause proliferation and differentation of osteoblasts and chondrocytes. The expression product of 608, or cells or vectors expressing 608 may cause cells to selectively proliferate and differentiate and thereby increase or alter bone density. Detecting levels of 608 mRNA or expression and comparing it to “normal” non-osteopathic levels may allow one to detect who may be at risk for osteoporosis or lower levels of osteoblasts and chondrocytes.

The medicament or treatment can be any conventional medicament or treatment for osteoporosis. Alternatively or additionally, the medicament or treatment can be the particular protein of the gene detected in the inventive methods, or that which inhibits that protein, e.g., binds to it. Similarly, additionally or alternatively, the medicament or treatment can be a vector which expresses the protein of the gene detected in the inventive methods or that which inhibits expression of that gene; again, for instance, that which can bind to it and/or otherwise prevents its transcription or translation. The selection of administering a protein or that which expresses it, or of administering that which inhibits the protein or the gene expression, can be done without undue experimentation, e.g., based on down regulation or up regulation as determined by inventive methods (e.g., in the osteoporosis model).

In the practice of the invention, one can employ general methods in molecular biology: Standard molecular biology techniques known in the art and not specifically described are generally followed as in Sambrook et al., Molecular Cloning: A Laboratory Manual, Cold Spring Harbor Laboratory, New York (1989, 1992), and in Ausubel et al., Current Protocols in Molecular Biology, John Wiley and Sons, Baltimore, Md. (1989).

PCR comprising the methods of the invention is performed in a reaction mixture comprising an amount, typically between <10 ng-200 ng template nucleic acid; 50-100 pmoles each oligonucleotide primer; 1-1.25 mM each deoxynucleotide triphosphate; a buffer solution appropriate for the polymerase used to catalyze the amplification reaction; and 0.5-2 Units of a polymerase, most preferably a thermostable polymerase (e.g., Taq polymerase or Tth polymerase).

Antibodies may be used in various aspects of the invention, e.g., in detection or treatment or prevention methods. Antibodies may be monoclonal, polyclonal or recombinant to be used in the immunoassays or other methods of analysis necessary for the practice of the invention. Conveniently, the antibodies may be prepared against the immunogen or portion thereof for example a synthetic peptide based on the sequence, or prepared recombinantly by cloning techniques or the natural gene product and/or portions thereof may be isolated and used as the immunogen. The genes are identified as set forth in the present invention and the gene product identified. Immunogens can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering - A Practical Guide, W. H. Freeman and Co., 1992. Antibody fragments may also be prepared from the antibodies and include Fab, F(ab1)2, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

For producing monoclonal antibodies the technique involves hyperimmunization of an appropriate donor with the immunogen, generally a mouse, and isolation of splenic antibody producing cells. These cells are fused to a cell having immortality, such as a myeloma cell, to provide a fused cell hybrid that has immortality and secretes the required antibody. The cells are then cultured, in bulk, and the monoclonal antibodies harvested from the culture media for use.

For producing recombinant antibodies (see generally Huston et al, 1991; Johnson and Bird, 1991; Mernaugh and Mernaugh, 1995), messenger RNAs from antibody producing β lymphocytes of animals, or hybridoma are reverse -transcribed to obtain complimentary DNAs (cDNAs). Antibody cDNA, which can be full or partial length, is amplified and cloned into a phage or a plasmid. The CDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnston & Thorpe, Immunochemistry in Practice, Blackwell Scientific Publications, Oxford, 1982.) The binding of antibodies to a solid support substrate is also well known in the art. See for a general discussion Harlow & Lane Antibodies: A Laboratory Manual, Cold Spring Harbor Laboratory Publications, N.Y., (1988) and Borrebaeck, Antibody Engineering - A Practical Guide, W. H. Freeman and Co., (1992). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹³C and iodination.

Antibodies can also be used as an active agent in a therapeutic composition and such antibodies can be humanized, for instance, to enhance their effects. See, Huls et al., Nature Biotech. 17:1999.

The expression product from the gene or portions thereof can be useful for generating antibodies such as monoclonal or polyclonal antibodies which are useful for diagnostic purposes or to block activity of expression products or portions thereof or of genes or a portion thereof, e.g., as a therapeutic. Monoclonal antibodies are immunoglobulins produced by hybridoma cells. A monoclonal antibody reacts with a single antigenic determinant and provides greater specificity than a conventional, serum-derived antibody. Furthermore, screening a large number of monoclonal antibodies makes it possible to select an individual antibody with desired specificity, avidity and isotype. Hybridoma cell lines provide a constant, inexpensive source of chemically identical antibodies and preparations of such antibodies can be easily standardized. Methods for producing monoclonal antibodies are well known to those of ordinary skill in the art, e.g. U.S. Pat. No. 4,196,265 and other documents cited herein, e.g., supra.

The genes of the present invention or portions thereof, e.g., a portion thereof which expresses a protein which function the same as or analogously to the full length protein, or genes identified by the methods herein can be expressed recombinantly, e.g., in E. coli or in another vector or plasmid for either in vivo expression or in vitro expression. The methods for making and/or administering a vector or recombinant or plasmid for expression of gene products of genes of the invention or identified by the invention or a portion thereof either in vivo or in vitro can be any desired method, e.g., a method which is by or analogous to the methods disclosed in: U.S. Pat. Nos. 4,603,112, 4,769,330, 5,174,993, 5,505,941, 5,338,683, 5,494,807, 4,722,848, WO 94/16716, WO 96/39491, Paoletti, Proc. Natl. Acad. Sci. USA 93:11349-11353 1996; Moss, Proc. Natl. Acad. Sci. USA 93:11341-11348 1996, Smith et al., U.S. Pat. No. 4,745,051 (recombinant baculovirus), Richardson, C. D. (Editor), Methods in Molecular Biology 39, “Baculovirus Expression Protocols” (1995 Humana Press Inc.), Smith et al., Mol. Cell. Biol. 1983 3: 2156-2165; Pennock et al., Mol. Cell. Biol. 1984 4:399-406; EPA 0 370 573, U.S. application Ser. No. 920,197, filed Oct. 16, 1986, EP Patent publication No. 265785, U.S. Pat. No. 4,769,331 (recombinant herpesvirus), Roizman, Proc. Natl. Acad. Sci. USA 93:11307-11312, Andreansky et al. Proc. Natl. Acad. Sci. USA 93:11313-11318; Robertson et al.; Proc. Natl. Acad. Sci. USA 93:11334-11340; Frolov et al. Proc. Natl. Acad. Sci. USA 93:11371-11377, October 1996, Kitson et al., J. Virol. 65:3068-3075, 1991; U.S. Pat. Nos. 5,591,439, 5,552,143 (recombinant adenovirus), Grunhaus et al., 1992, Sem. Virol. 3: 237-52, 1993, Ballay et al. EMBO J. 4:3861-65, Graham, Tibtech 8:85-87 1990, Prevec et al., J. Gen Virol. 70:429-434, PCT WO91/11525, Felgner et al. (1994), J. Biol. Chem. 269:2550-2561, Science, 259:1745-49, 1993 and McClements et al.; Proc. Natl. Acad. Sci. USA 93:11414-11420 1996, and U.S. Pat. Nos. 5,591,639, 5,589,466, and 5,580,859 relating to DNA expression vectors, inter alia. See also WO 98/33510; Ju et al., Diabetologia, 41:736-739, 1998 (lentiviral expression system); Sanford et al., U.S. Pat. No. 4,945,050 (method for transporting substances into living cells and tissues and apparatus therefor); Fischbach et al. (Intracel), WO 90/01543 (method for the genetic expression of heterologous proteins by cells transfected); Robinson et al., seminars in IMMUNOLOGY, vol. 9, pp.271-283 (1997) (DNA vaccines); Szoka et al., U.S. Pat. No. 4,394,448 (method of inserting DNA into living cells); and McCormick et al., U.S. Pat. No. 5,677,178 (use of cytopathic viruses for therapy and prophylaxis of neoplasia).

The expression product generated by vectors or recombinants in this invention optionally can also be isolated and/or purified from infected or transfected cells; for instance, to prepare compositions for administration to patients. However, in certain instances, it may be advantageous to not isolate and/or purify an expression product from a cell; for instance, when the cell or portions thereof enhance the effect of the polypeptide.

An inventive vector or recombinant expressing a gene identified herein or from a method herein or a portion thereof can be administered in any suitable amount to achieve expression at a suitable dosage level, e.g., a dosage level analogous to the herein mentioned dosage levels (wherein the gene product is directly present). The inventive vector or recombinant can be administered to a patient or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu. In plasmid compositions, the dosage should be a sufficient amount of plasmid to elicit a response analogous to compositions wherein gene product or a portion thereof is directly present; or to have expression analogous to dosages in such compositions; or to have expression analogous to expression obtained in vivo by recombinant compositions. For instance, suitable quantities of plasmid DNA in plasmid compositions can be 1 μg to 100 mg, preferably 0.1 to 10 mg, e.g., 500 micrograms, but lower levels such as 0.1 to 2 mg or preferably 1-10 μg may be employed. Documents cited herein regarding DNA plasmid vectors can be consulted for the skilled artisan to ascertain other suitable dosages for DNA plasmid vector compositions of the invention, without undue experimentation.

Compositions for administering vectors can be as in or analogous to such compositions in documents cited herein or as in or analogous to compositions herein described, e.g., pharmaceutical or therapeutic compositions and the like (e.g., see infra).

Thus, the invention comprehends in vivo gene expression which is sometimes termed “gene therapy”. Gene therapy can refer to the transfer of genetic material (e.g DNA or RNA) of interest into a host to treat or prevent a genetic or acquired disease or condition phenotype. The particular gene that is to be used or which has been identified as the target gene is identified as set forth herein. The genetic material of interest encodes a product (e.g. a protein, polypeptide, peptide or functional RNA) whose production in vivo is desired. For example, the genetic material of interest can encode a hormone, receptor, enzyme, polypeptide or peptide of therapeutic value. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, homologous recombination, etc.) and, an expression system as needed and then the modified cells are expanded in culture and returned to the host/patient. These genetically reimplanted cells have been shown to produce the transfected gene product in situ. In in vivo gene therapy, target cells are not removed from the subject rather the gene to be transferred is introduced into the cells of the recipient organism in situ, that is within the recipient. Alternatively, if the host gene is defective, the gene is repaired in situ (Culver, 1998. These genetically altered cells have been shown to produce the transfected gene product in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore as used herein the expression vehicle may, as needed, not include the 5′UTR and/or 3′UTR shown in sequences herein and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene as described herein.

Vectors can be introduced into cells or tissues by any one of a variety of known methods within the art. Such methods can be found generally described in Sambrook et al., Molecular Cloning: A Laboratory Manual, Cold Springs Harbor Laboratory, N.Y. (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al (1986), as well as other documents cited herein (see supra) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur. Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Delivery of gene products (products from herein defined genes: genes identified herein or by inventive methods or portions thereof) and/or antibodies or portions thereof and/or agonists or antagonists (collectively or individually “therapeutics”), and compositions comprising the same, as well as of compositions comprising a vector expressing gene products, can be done without undue experimentation from this disclosure and the knowledge in the art.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators, e.g., of osteoporosis, for instance, improvement in bone density, as are selected as appropriate measures by those skilled in the art.

It is noted that humans are treated generally longer than the mice or other experimental animals exemplified herein which treatment has a length proportional to the length of the disease process and drug effectiveness. The doses may be single doses or multiple doses over a period of several days, but single doses are preferred. Thus, one can scale up from animal experiments, e.g., rats, mice, and the like, to humans, by techniques from this disclosure and the knowledge in the art, without undue experimentation.

The present invention provides an isolated nucleic acid molecule comprising nucleotides having a sequence set forth in SEQ ID NO:1, SEQ ID NO:3, SEQ ID NO:6 or SEQ ID NO:20, supplements thereof and a polynucleotide having a sequence that differs from SEQ ID NO:1, SEQ ID NO:3, SEQ ID NO:6 or SEQ ID NO:20 due to the degeneracy of the genetic code or a functional portion thereof or a polynucleotide which is at least substantially homologous or identical thereto. In a prefered embodiment, the nucleic acid molecule comprises a polynucleotide having at least 15 nucleotides from SEQ ID NO:1, SEQ ID NO:3, SEQ ID NO:6 or SEQ ID NO:20, prefereably at least 50 nucleotides and more preferably at least 100 nucleotides.

The present invention also provides a composition comprising the isolated nucleic acid molecule, a vector comprising the isolated nucleic acid molecule of claim, a composition comprising said vector and a method for preventing, treating or controlling osteoporosis, osteopenia, osteopetrosis, osteosclerosis, osteoarthritis, periodontosis, bone fractures or low bone density or or other conditions involving mechanical stress or a lack thereof in a subject, comprising administering the inventive composition, or the inventive vector, and a method for preparing a polypeptide comprising expressing the isolated nucleic acid molecule or comprising expressing the polypeptide from the vector.

The present invention further provides a method for preventing, treating or controlling osteoporosis, osteopenia, osteopetrosis, osteosclerosis, osteoarthritis, periodontosis, bone fractures or low bone density or other factors causing or contributing to osteoporosis or symptoms thereof or other conditions involving mechanical stress or a lack thereof in a subject, comprising administering an isolated nucleic acid molecule or functional portion thereof or a polypeptide comprising an expression product of the gene or functional portion of the polypeptide or an antibody to the polypeptide or a functional portion of the antibody. In one embodiment of the invention, the isolated nucleic acid molecule encodes a 10 kD to 30 kD N-terminal cleavage product of the 608 protein, preferably, the N-terminal cleavage product comprises of a polypeptide of about 25 kD.

The present invention provides an isolated polypeptide encoded by the inventive polynucleotide. In one embodiment of the invention, the polypeptide is identified as protein 608 or a functional portion thereof or a polypeptide which is at least substantially homologous or identical thereto. In another embodiment of the invention, the polypeptide is a human protein 608 or a functional portion thereof. Preferably, the functional portion comprises a N-terminal polypeptide having a molecular weight of 10 kD to 30 kD. More preferably, the the functional portion comprises an N-terminal polypeptide having a molecular weight of about 25 kD.

The present invention also provides a composition comprising one or more isolated polypeptides, an antibody elicited by the polypeptide or a functional portion thereof, a composition comprising the antibody or a functional portion thereof, and a method for treating or preventing osteoporosis, or for fracture healing, bone enlongation, or periodontosis in a subject, comprising administering to the subject a N-terminal polypeptide having a molecular weight of between 10 kD and 30 kD, preferably about 25 kD.

The present invention provides for a method of treating or preventing osteoarthritis, osteopetrosis, or osteosclerosis, comprising administering to a subject an effective amount of a chemical or a neutralizing monoclonal antibodies which inhibit the activity of the N-terminal polypeptide having a molecular weight of between 10 kD and 30 kD, preferably about 25 kD.

As used herein, the term “subject” includes, but not limited to human, bovine, pig, mouse, rat, goat, sheep and horse.

Those skilled in the art will recognize that the components of the compositions should be selected to be chemically inert with respect to the gene product and optional adjuvant or additive. This will present no problem to those skilled in chemical and pharmaceutical principles, or problems can be readily avoided by reference to standard texts or by simple experiments (not involving undue experimentation), from this disclosure and the documents cited herein.

A better understanding of the present invention and of its many advantages will be had from the following examples, given by way of illustration and as a further description of the invention.

### EXPERIMENTAL DETAILS

TGF-β1 is known as a principal inducer of connective tissue growth factor (CTGF, cef10, fisp12, cyr61, βIG-M1, β IG-M2, non-protooncogene) expression. The latter contains four distinct structural modules, each of them being homologous to distinct domains in other extracellular proteins such as Von Willebrand factor, slit, thrombospondins, fibrillar collagens, IGF-binding proteins and mucins. CTGF expression is induced not only by TGF-↑1, but also by BMP2 (bone morphogenic factor 2), and during wound repair. In embryogenesis, its expression is found in developing cartilaginous elements, including limbs, ribs, prevertebrae, chondrocranium and craniofascial elements (Meckel's cartilage). Thus, CTGF transcription correlates with differentiation of chondrocytes of both mesodermal and ectodermal origin. In culture, CTGF is expressed in chondrocytes but not in osteoblasts. Possible role in endochondral ossification is suspected because of responsiveness to BMP2. In fibroblasts, CTGF expression causes upregulation of alpha-1-collagen, alpha-5-integrin and fibronectin.

### EXAMPLE 1

### CMF608 Gene Expression by In Situ Hybridization

The pattern of expression of CMF608 gene was studied by in situ hybridization on sections of bones from ovariectomized and sham-operated rats. Female Wistar rats weighting 300-350 g were subjected to ovariectomy under general anesthesia. Control rats were operated in the same way but ovaries were not excised - sham operation.

Three weeks after the operation rats were sacrificed and tibia were excised together with the knee joint. Bones were fixed for three days in 4% paraformaldehyde and then decalcified for four days in solution containing 5% formic acid and 10% formalin. Decalcified bones were postfixed in 10% formalin for three days and embedded into paraffin.

To study the pattern of expression of the CMF608 gene in bone development, the model of ectopic bone formation was employed. Rat bone marrow cells were seeded into cylinders of demineralized bone matrix prepared from rat tibiae. Cylinders were implanted subcutaneously into adult rats. After three weeks rats were sacrificed and implants were decalcified and embedded into paraffin as described above for tibial bones.

The 6 μm sections were prepared and subjected to in situ hybridization procedure. After hybridization sections were dipped into nuclear track emulsion and exposed for three weeks at 4° C. Autoradiographs were developed, stained with hematoxylin-eosin and studied under microscope using brightfield and darkfield illumination.

For further assessment of cell and tissue specificity of CMF608 gene expression, in situ hybridization study was performed on sections of multitissue block containing multiple samples of adult rat tissues. The developmental pattern of CMF608 expression was studied on sagittal sections of mouse embryos of 12.5, 14.5 and 16.5 days postconception (dpc) stages.

Microscopic study of hybridized sections of long bones revealed a peculiar pattern of CMF608 probe hybridization. The hybridization signal can be seen mainly in fibroblast-like cells found in several locations throughout the sections. Prominent accumulations of these cells can be seen in the area of periosteal modeling in metaphysis, and also in regions of active remodeling of compact bone in diaphysis: at the boundary between bone marrow and endosteal osteoblasts and in periosteum, also in close contact with osteoblasts. Perivascular connective tissue filling Volkmann's canals in compact bone in diaphysis and epiphysis also contains expressing cells. No hybridization is found within cancellous bone and in bone marrow. This pattern of hybridization suggests that cells showing expression of CMF608 are associated with areas of remodeling of preexisting bone and are not involved in primary endochondral ossification.

At the growth plate level, expressing cells can be seen in the perichondral fibrous ring of LaCroix. Some investigators regard this fibrous tissue as the aggregation of residual mesenchymal cells able to differentiate into both osteoblasts and chondrocytes. In this respect it is noteworthy that single cells expressing CMF608 can be seen in epiphyseal cartilage. These expressing cells are rounded cells within the lateral segment of epiphysis (sometimes in close vicinity to the LaCroix ring) and flattened cells covering the articulate surface. Most cells in articulate cartilage and all chondrocytes on the growth plate do not show expression of CMF608. Ovariectomy did not alter the intensity and pattern of CMF608 expression in bone tissue.

In ectopic bone sections, hybridization signal for CMF608 can be seen in some fibroblast-like cells either scattered within unmineralized connective tissue matrix or concentrated at the boundary between this tissue and osteoblasts of immature bone.

Pattern of expression of CMF608 gene revealed by in situ hybridization in bone and cartilage allows to speculate that its expression marks some skeletal tissue elements able to differentiate into two skeletal cell types—osteoblasts and chondrocytes. The terminal differentiation of these cells appears to be accompanied by down-regulation of CMF608 expression. The latter suggestion is supported by peculiar temporal pattern of CMF608 expression in primary cultures of osteogenic cells isolated from calvaria bones of rat fetuses. In these cultures expression was revealed by in situ hybridization in vast majority of cells after one and two weeks of incubation in vitro. Three and four week old cultures showing signs of ossification contain no expressing cells. Significantly, no hybridization signal was found on sections of multitissue block hybridized to CMF608 probe suggesting high specificity of this gene expression for the skeletal tissue in adult organism.

In situ hybridization study of embryonic sections demonstrated that at 12.5 dpc weak hybridization signal can be discerned in some mesenchymal cells in several locations throughout the embryonic body. The most prominent signal is found in the head: in loose mesenchymal tissue surrounding the olfactory epithelium and underlying the surface epithelium of nose tip. Other mesenchymal cells in the head also show hybridization signal: in non-cartilaginous part of basisphenoid bone primordium and in mesenchyme surrounding the dental laminae (tooth primordia) in the mandible.

In the trunk, expression can be detected in less developed vertebrae primordia in the thoraco-lumbar region. The hybridization signal here marks the condensed portion of sclerotomes. Another area of the trunk showing hybridization signal is comprised of a thin layer of mesenshymal cells in the anterior part of thoracic body wall.

At later stages of development -14.5 and 16.5 dpc probe CMF608 gave no hybridization signal. Thus, it appears that during embryonic development CMF608 gene is transiently expressed by at least some mesenchymal and skeleton-forming cells. This expression is down-regulated at later stages of development. More detailed study of late embryonic and postnatal stages of development reveals the timing of appearance of CMF608 expressing cells in bone tissue.

### EXAMPLE 2

### Isolation of Rat OCP

In order to search for a stimulator of bone formation following mechanical force, the inventors used primary rat calvaria cells grown on elastic membranes that were stretched for 20 minutes. Genes expression patterns were compared before and after the application of mechanical force.

The expression of the novel gene OCP was found to be upregulated approximately 3-fold by mechanical force. This was detected both by microarray analysis and by Northern blot analysis using poly (A)+ RNA from rat calvaria cells before and after the mechanical stress. In rat calvaria primary cells and in rat bone extract this gene was expressed as a main RNA species of approximately 8.9 Kb and a minor RNA transcript of approximately 9 Kb. The hybridization signal was not detected in any other rat RNA from various tissue sources, including testis, colon, intestine, kidney, stomach, thymus, lung, uterus, heart, brain, liver, eye, and lymph node (data not shown).

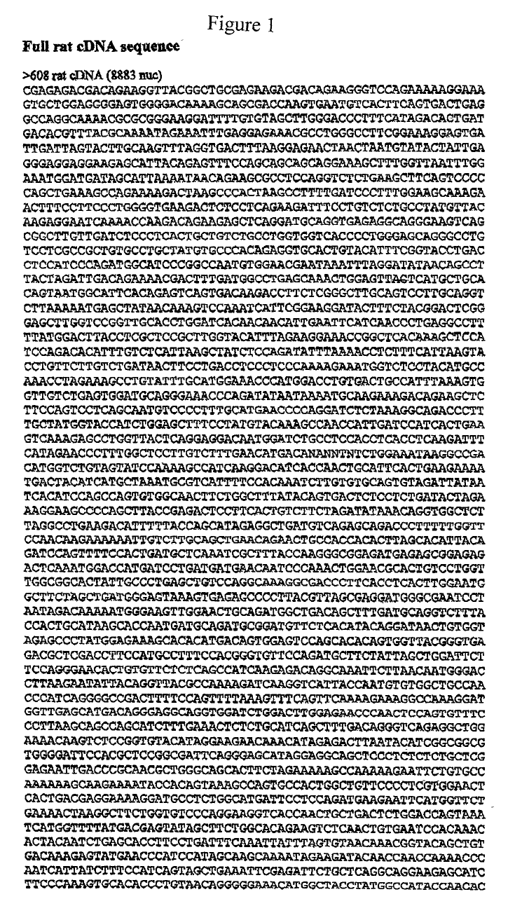

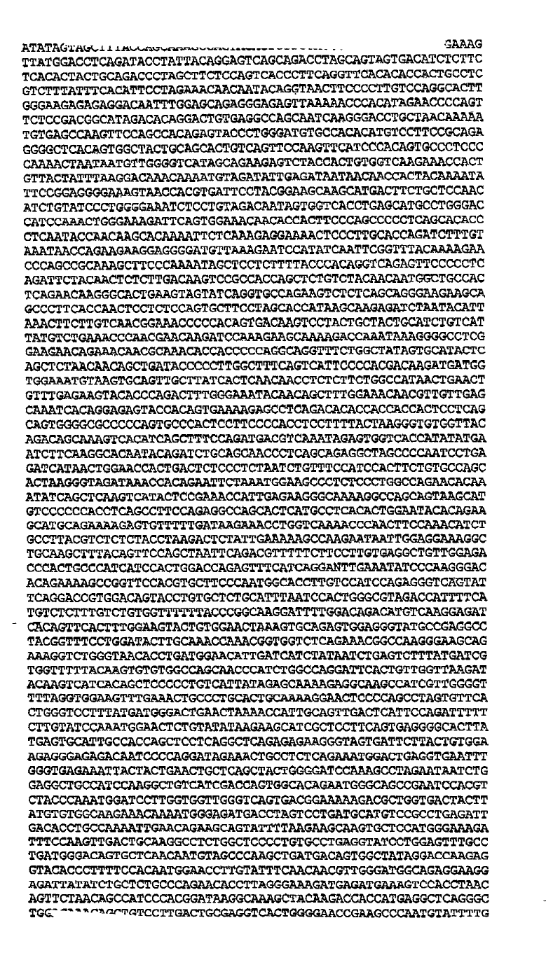

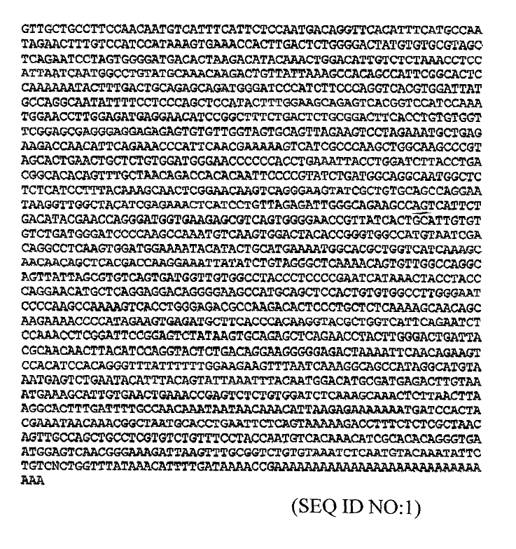

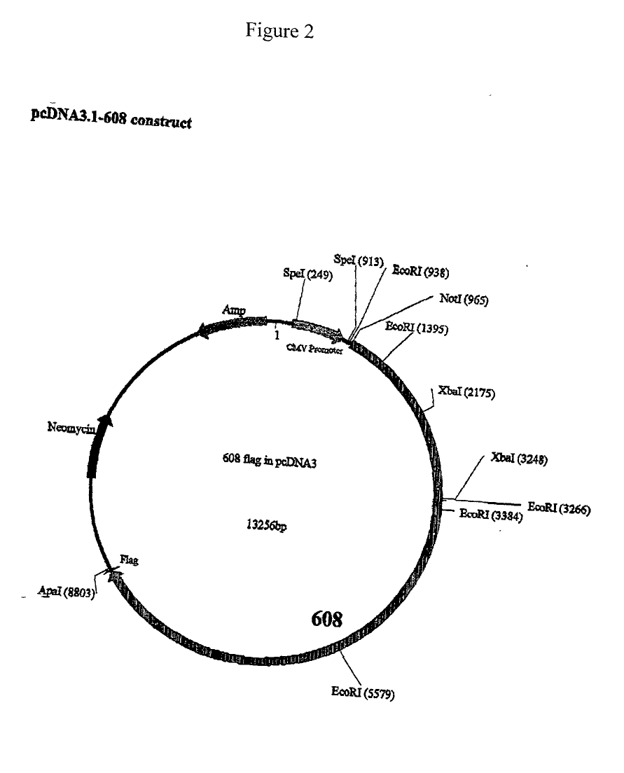

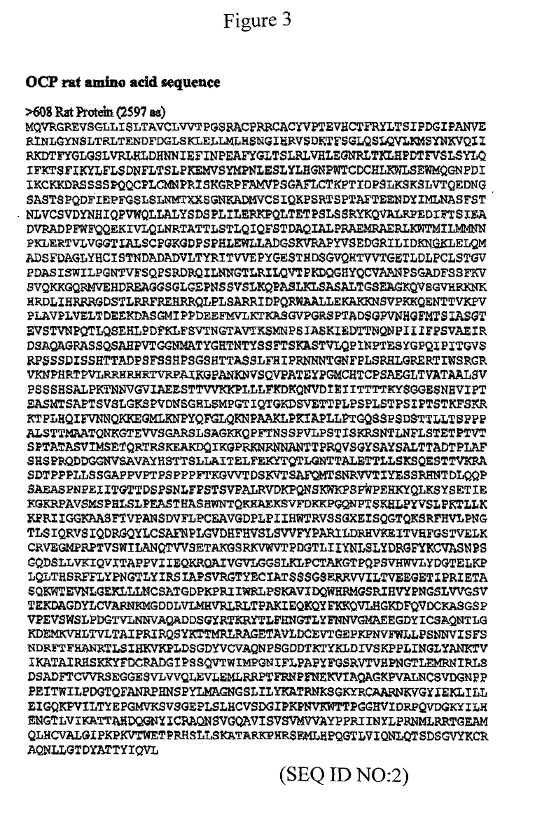

The partial OCP rat cDNA clone ( 4007 bp long) that was isolated from a rat calvaria cDNA phage library was found to contain a 3356 bp open reading frame closed at the 3′ end. Comparison to public mouse databases revealed no sequence homologues. A complete OCP rat cDNA clone was isolated from the rat calvaria cDNA library by a combination of 5′ RACE technique (clontech), RT-PCR of 5′ cDNA fragments and ligation of the latter products to the original 3′ clone. The full rat cDNA clone that was generated (shown in FIG. 1 and pCDNA3.1-608, in FIG. 2) was sequenced, and no mutations were found. The full sequence stretch is 8883 bp long and contains an ORF (nt 575-8366) for a 2597 amino acid protein (shown in FIG. 3). The CDNA does not contain a polyadenylation site, but holds a 3′ poly A stretch.

The inventors found that CMF608 encodes a large protein that is most probably a part of the extra-cellular matrix. The gene may be actively involved in supporting osteoblast differentiation. Another option is that it marks regions were remodeling takes place. Such an hypothesis is also compatible with a role in directing osteoclast action and thus it may be a target for inhibition by small molecules.

In normal bone formation, activation of osteoblasts leads to secretion of various factors that attract osteoclast precursors or mature osteoclasts to the sites of bone formation to initiate the process of bone resorption. In normal bone formation both functions are balanced. Imbalance to any side causes either osteoporosis (osteoblast function overwhelms) or osteoporosis (osteoclast function overwhelms).

Among known osteoblast activators—mechanical force stimulation—is actually applied in the present model. As proof of principle, increased expression of several genes known to respond to mechanical stress by transcriptional upregulation were found. They include tenascin, endothelin and possibly trombospondin. Upregulation of water channel encoding message is likely related to this mechanism too.

### EXAMPLE 3

### Full-Length OCP cDNA Construction and Expression

TNT (transcription - translation) assays were performed as described (Promega - TNT coupled reticulocyte lysate systems), using specific fragments taken from various regions of the gene. The following fragments were tested:

TABLE 1
TNT products

| | | Size of fragment | Size of translation | Promoter |
|---|---|---|---|---|
| Frg. | Location | (bp) | product (kD) | used |
| 1 | 134-2147 | 2013 | 73 | T7 |
| 2 | 3912-5014 | 1102 | 40 | ″ |
| 3 | 574-1513 | 939 | 34 | ″ |

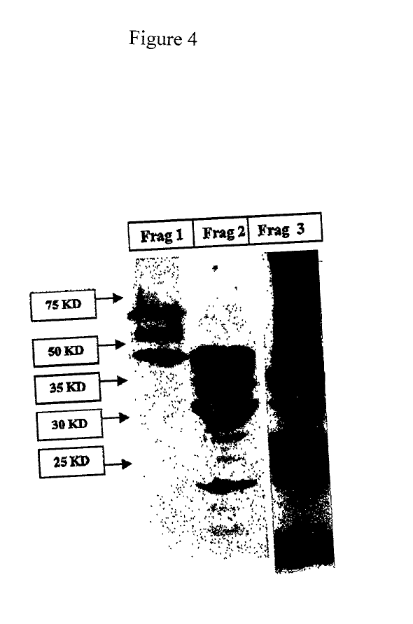

In all assays a clear translation product was observed (see FIG. 4).

### EXAMPLE 4

### The Mouse OCP Gene

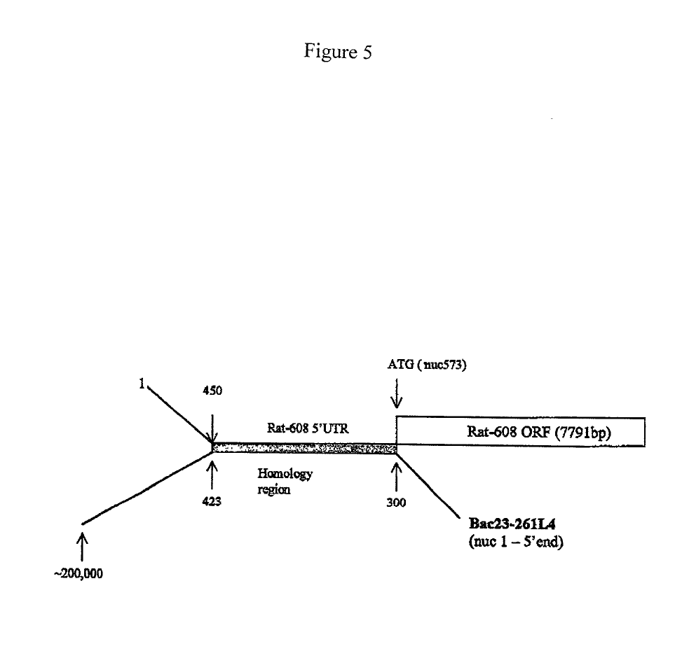

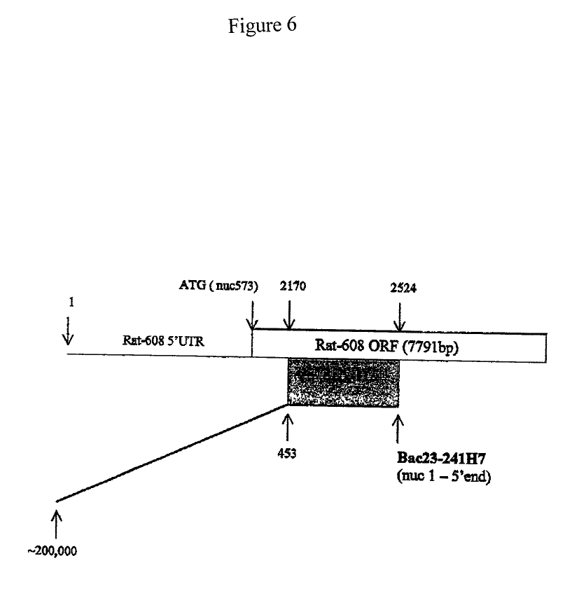

Two mouse genomic Bac clones containing the mouse OCP gene promoter region and part of the coding region were identified, based on their partial homology to the 5′UTR region of the rat-608 cDNA. These clones (23-261L4 and 23-241H7 with ˜200Kb average insert length) were bought from TIGR (FIGS. 5 & 6).

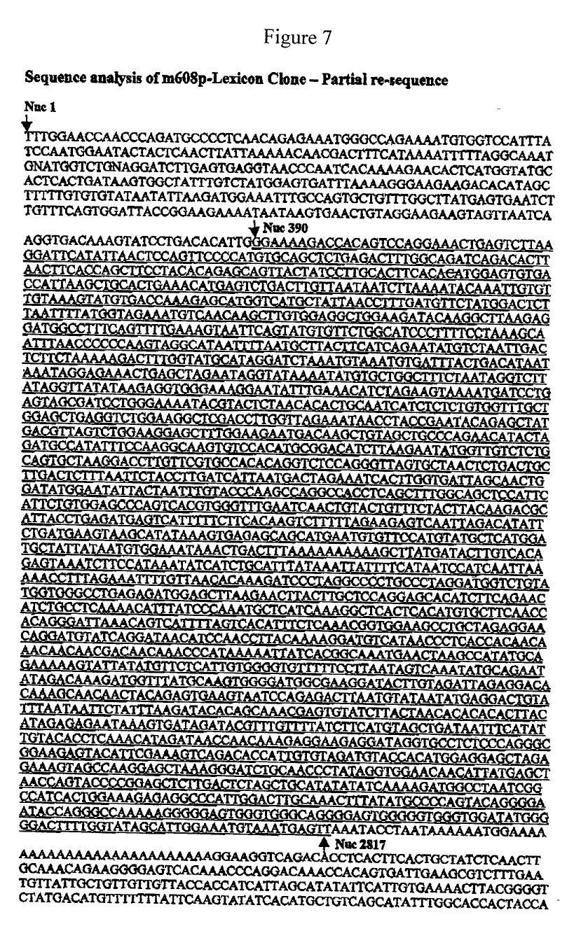

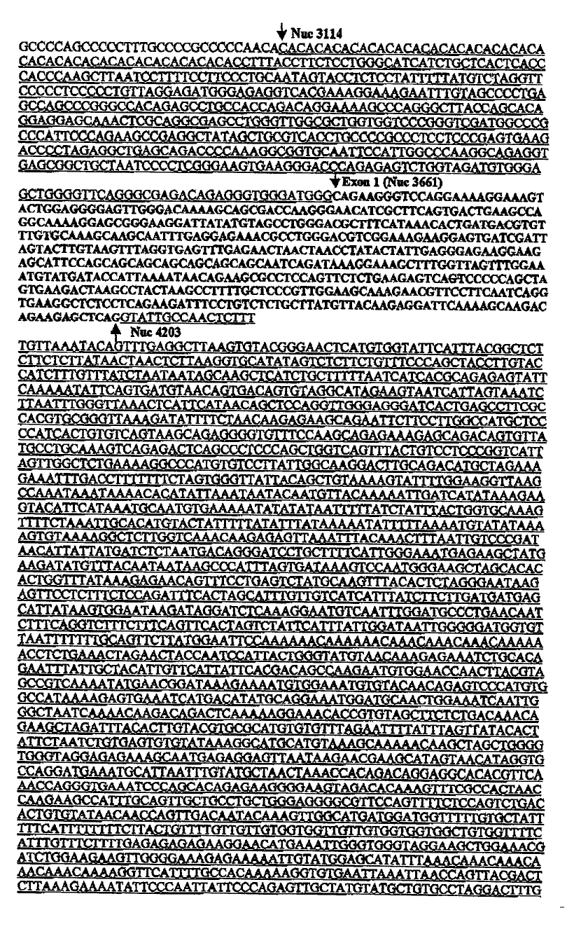

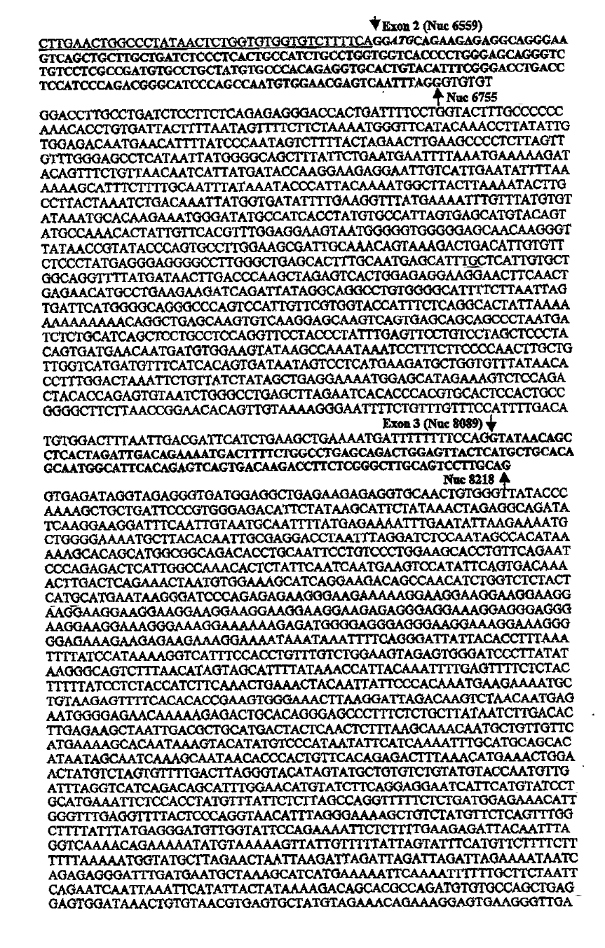

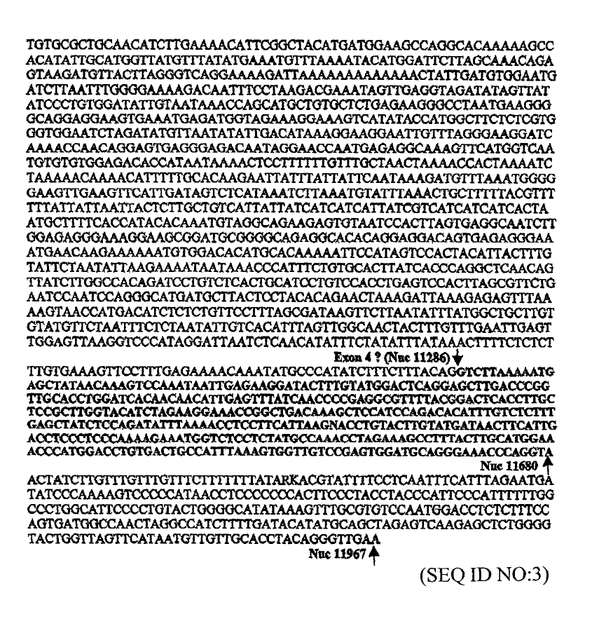

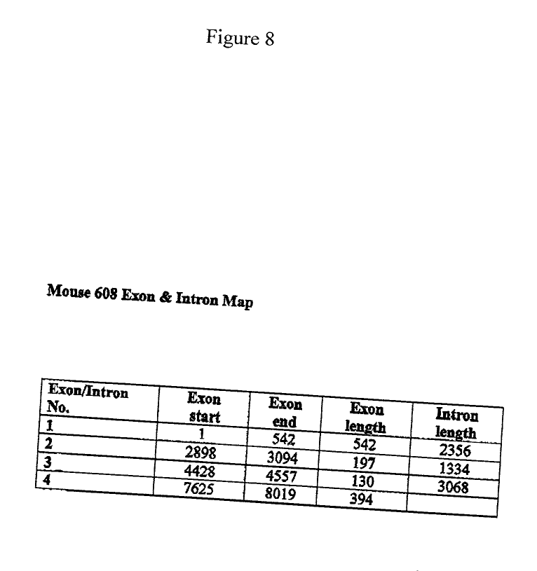

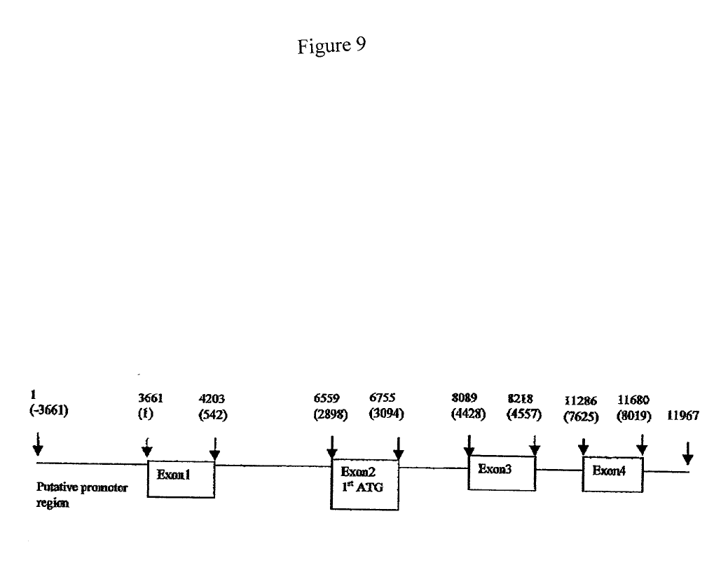

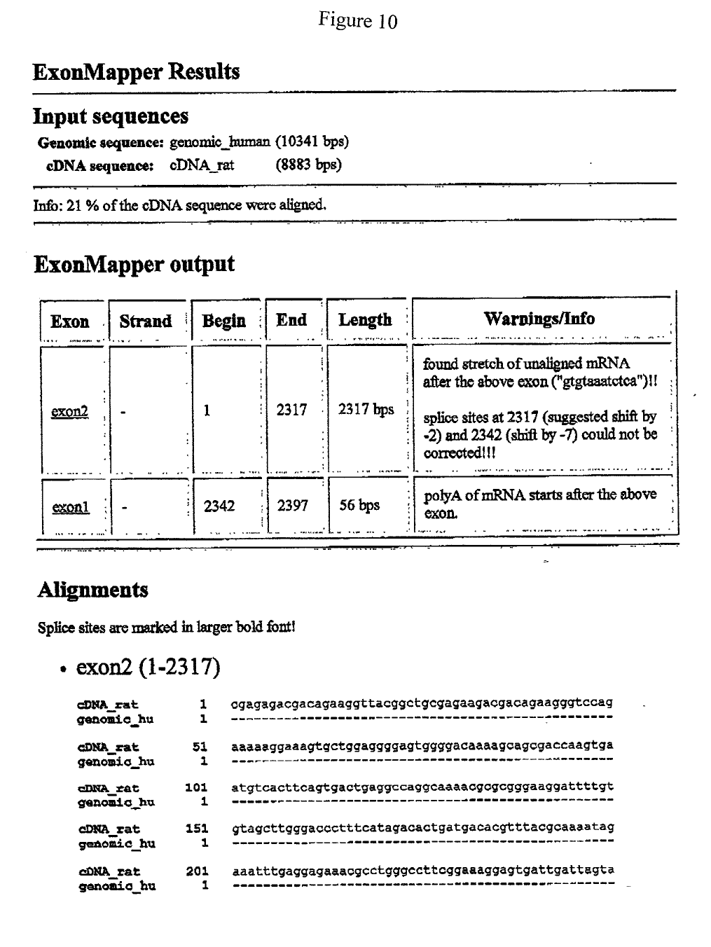

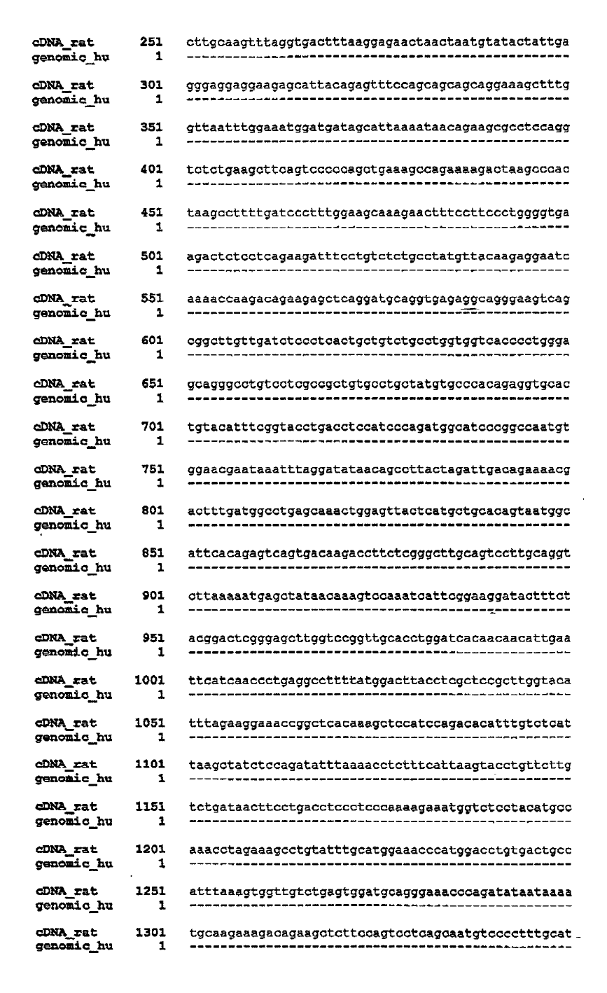

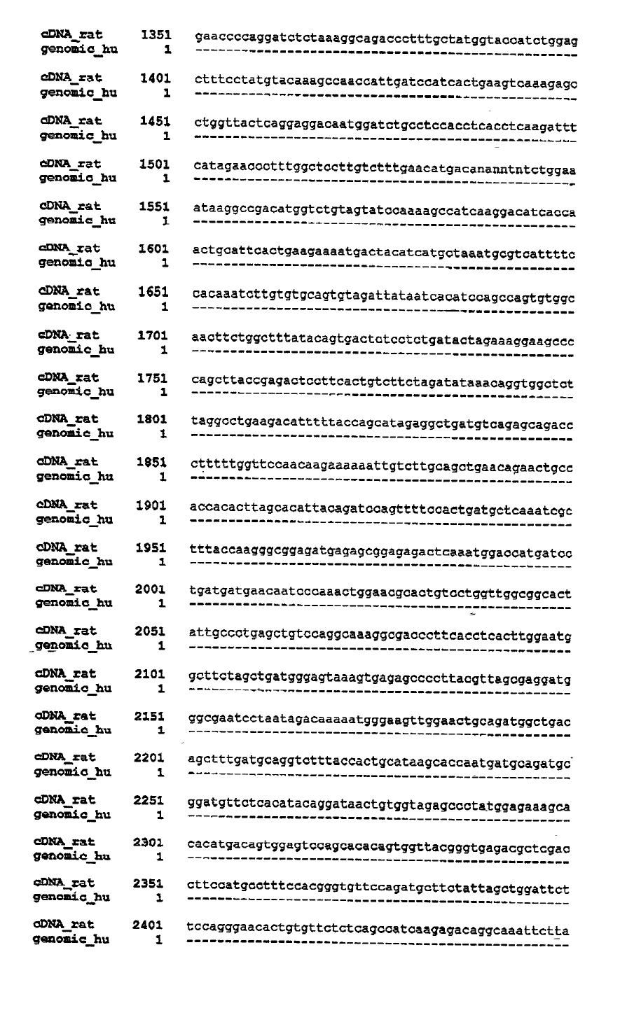

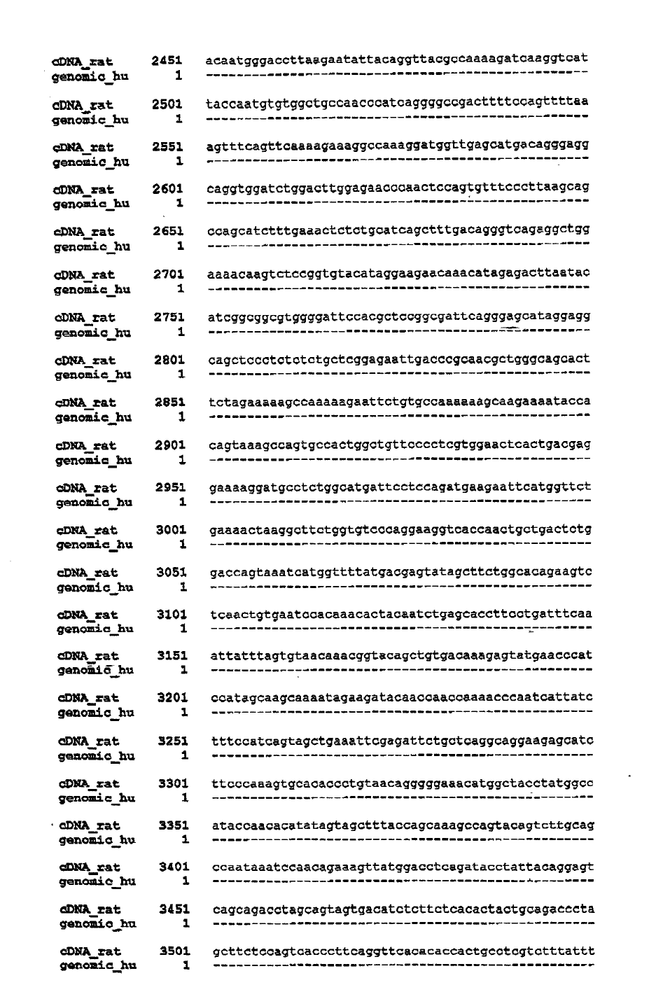

Specific primers for the amplification of a part of the mouse-OCP promoter region were designed and used for PCR screening of a mouse genomic phage library (performed by Lexicon Genetics Inc. for the Applicants). One phage clone containing part of the genomic region of the mouse 608 gene was detected and completely sequenced. The length of this clone was reported to be 11,963 bp. Parts of the physical “Lexicon” clone were re-sequenced by the inventors and corrections were made. The resequenced clone (shown in FIG. 7) is 11967 bp long. Exon-location prediction (shown in FIG. 8) was performed by the Applicant company's Bioinformatics unit based on the alignment of the mouse genomic and the rat cDNA sequences (FIGS. 9 and 10, respectively).

### EXAMPLE 5

### The Human OCP Gene

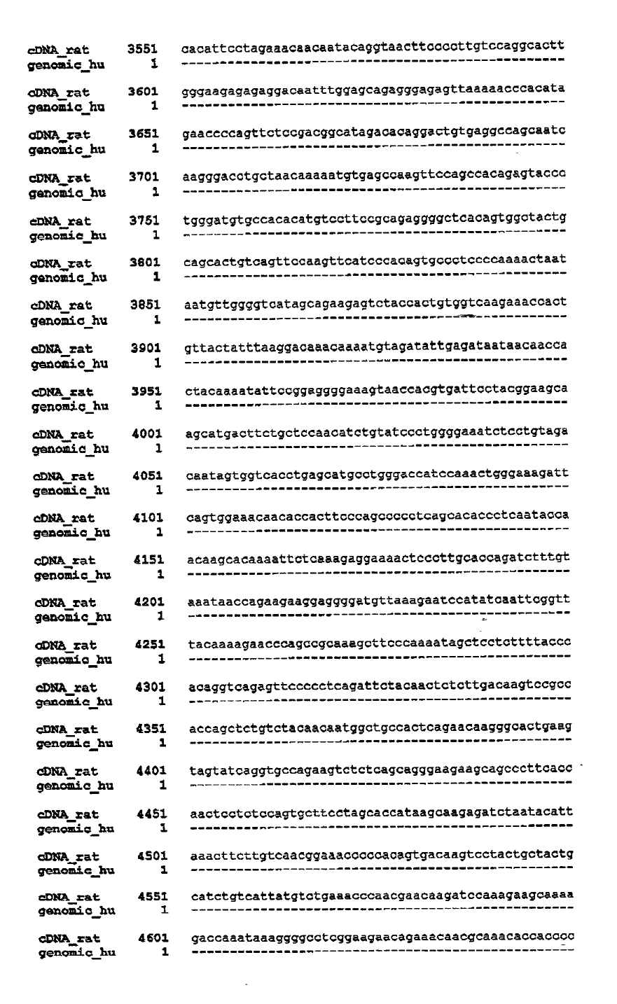

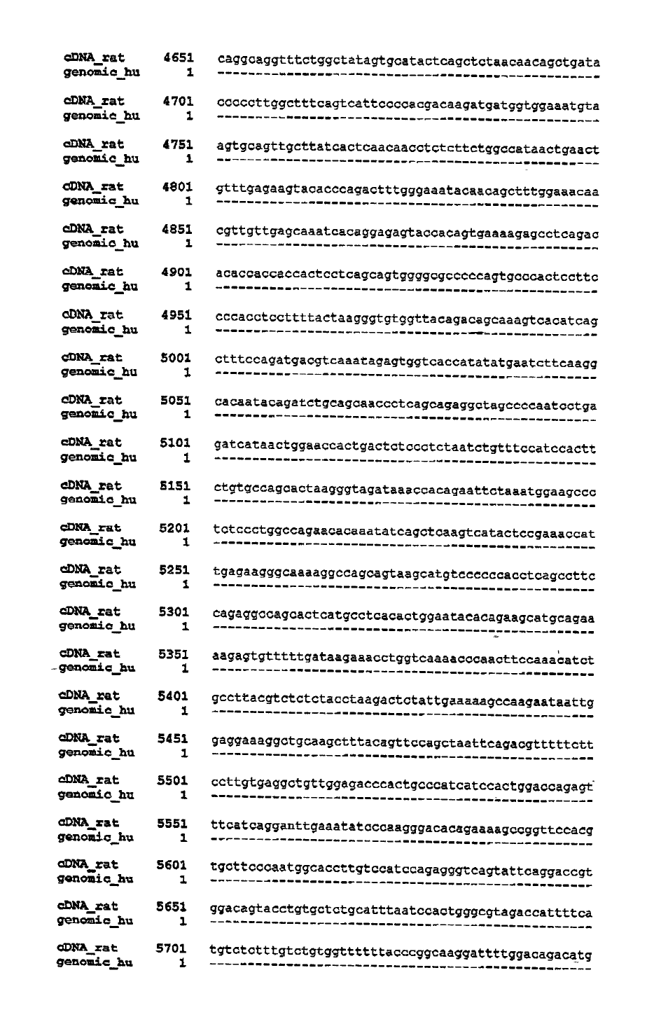

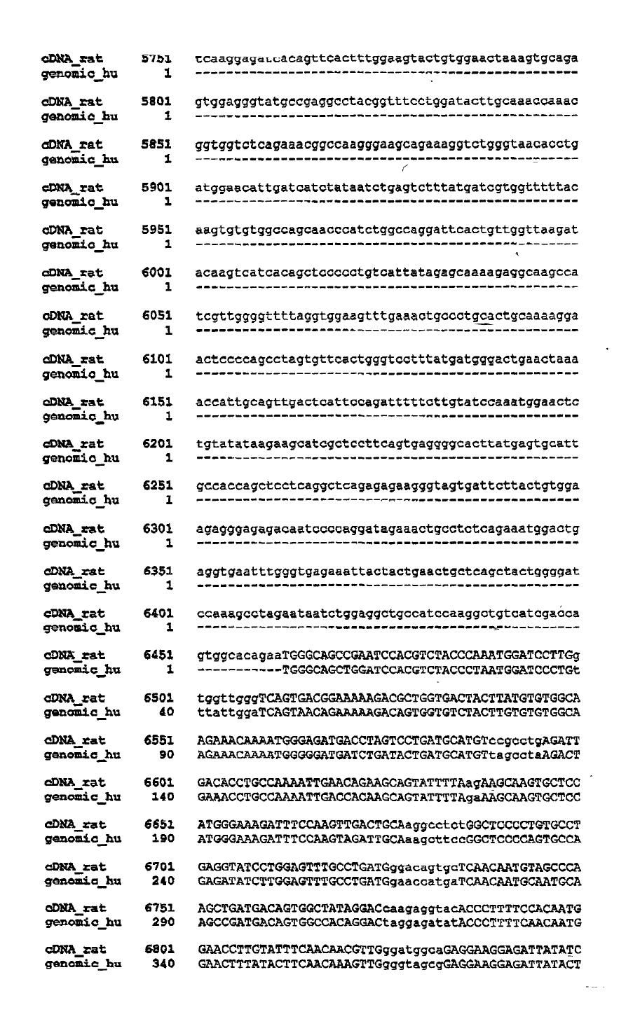

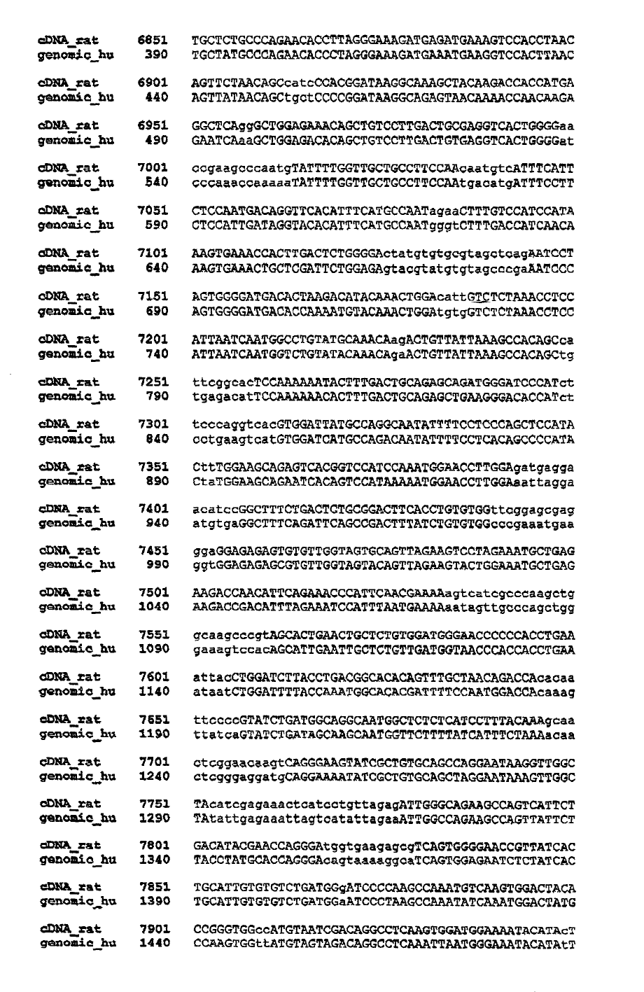

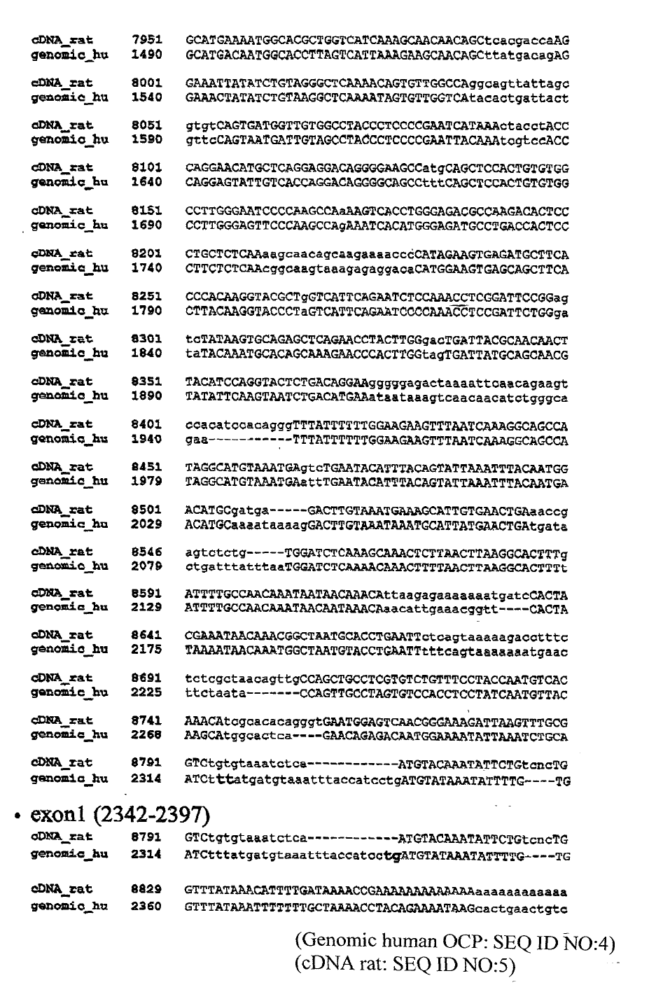

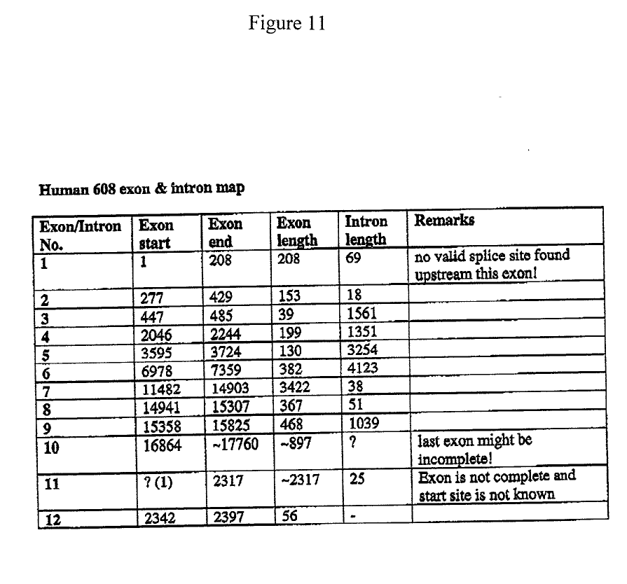

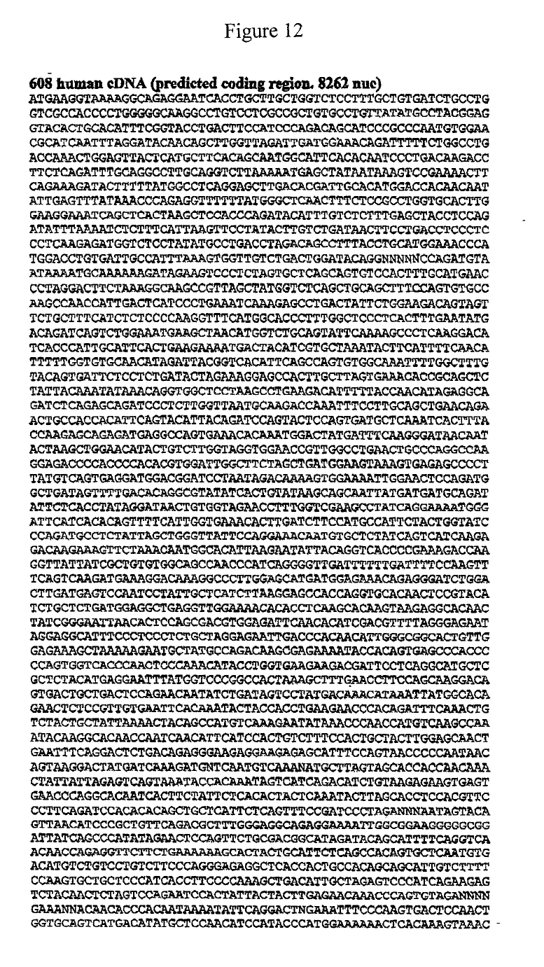

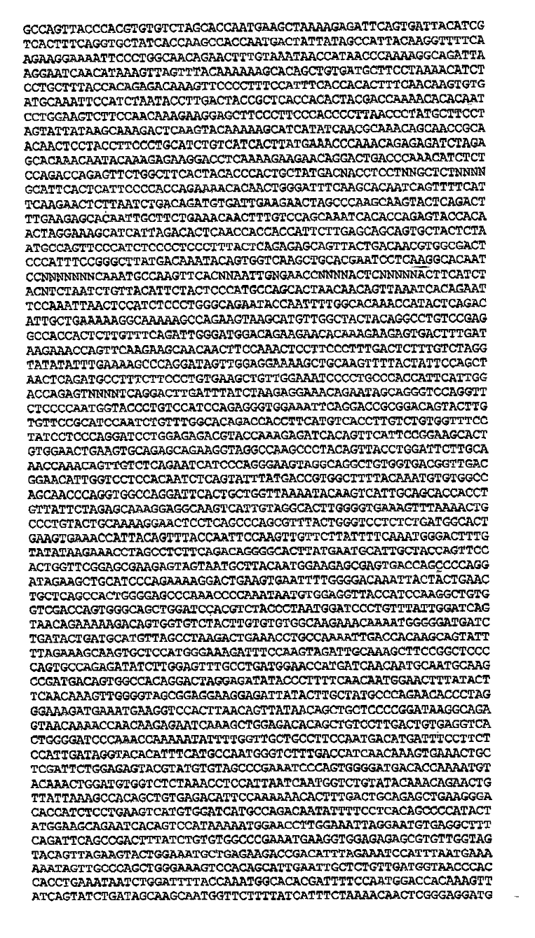

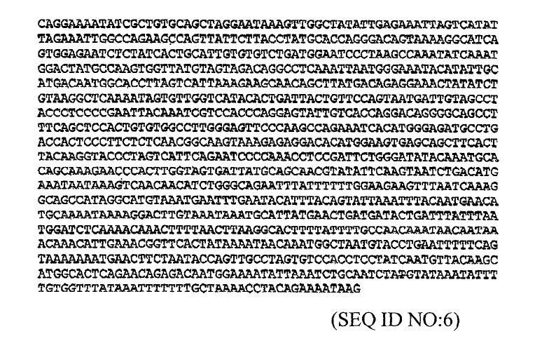

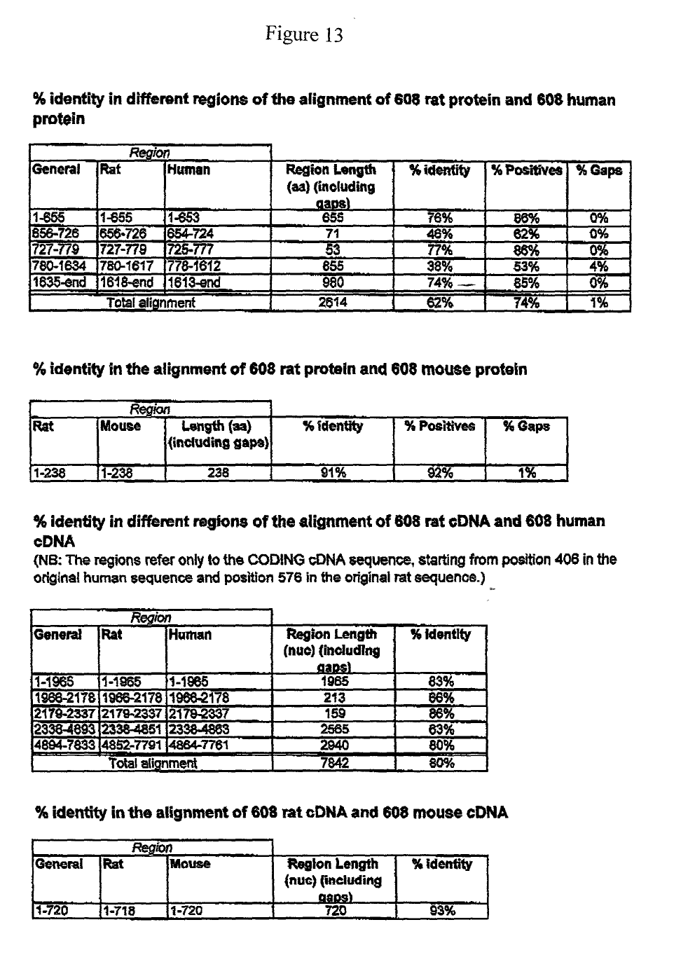

On the nucleotide level, the rat OCP cDNA sequence is homologous to the human genomic DNA sequence located on chromosome 3. Based on the homology and bioinformatic analysis (shown in FIGS. 10 and 11), a putative cDNA sequence was generated (FIG. 12). The highest similarity is evident between nt 1-1965 (1-655 a.a), 2179-2337 (727-779 a.a) and 4894-7833 (1635 a.a.-end) as presented in the table shown in FIG. 13. On the protein level, no homologues were found in the data bank.

### EXAMPLE 6

### The Deduced OCP Protein

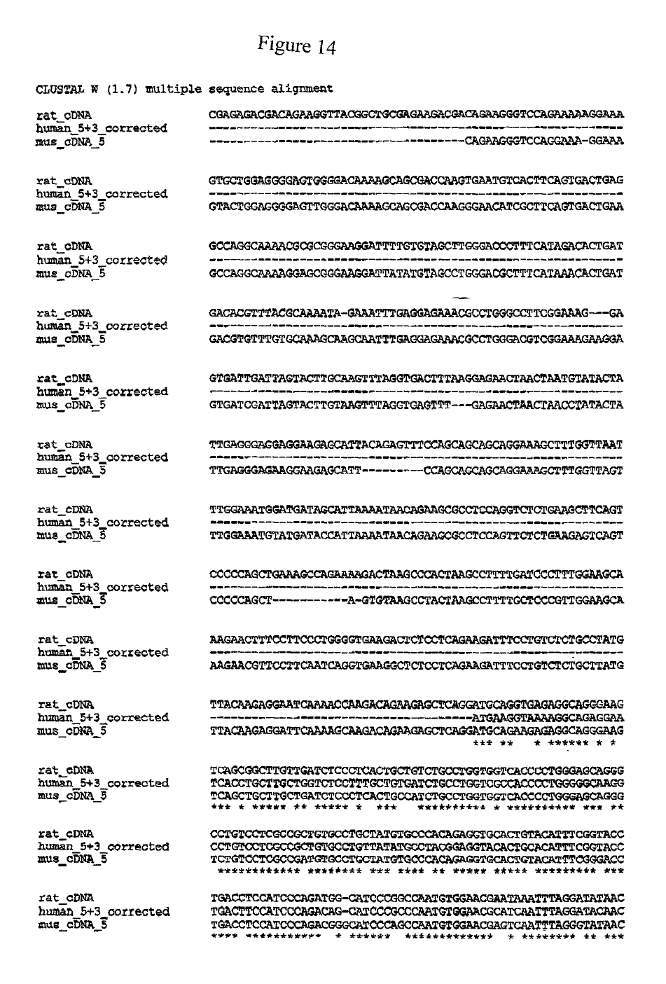

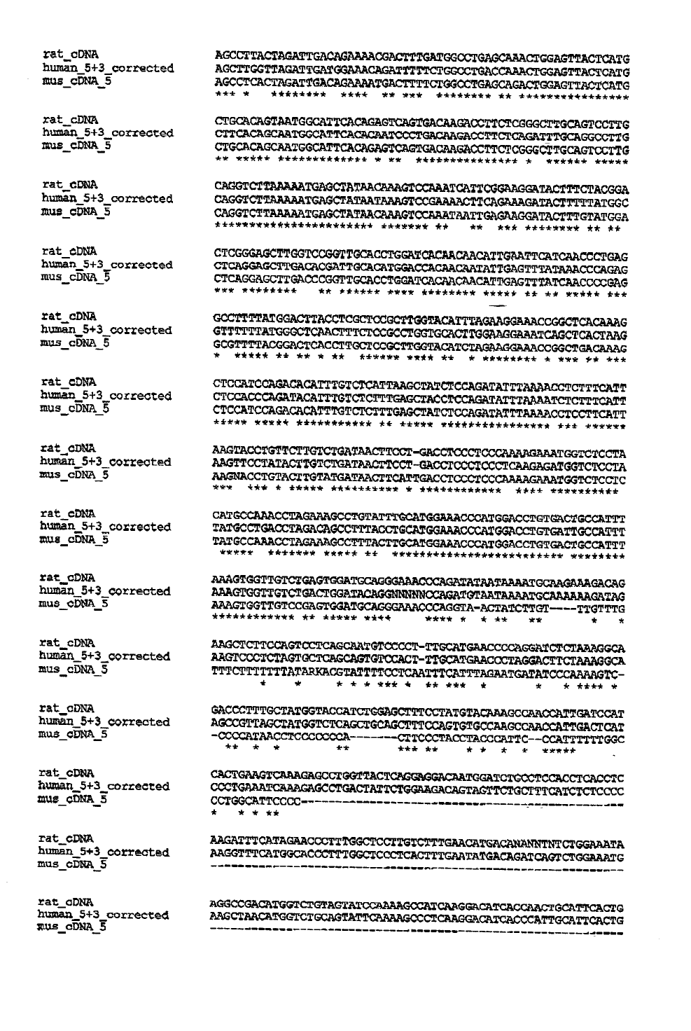

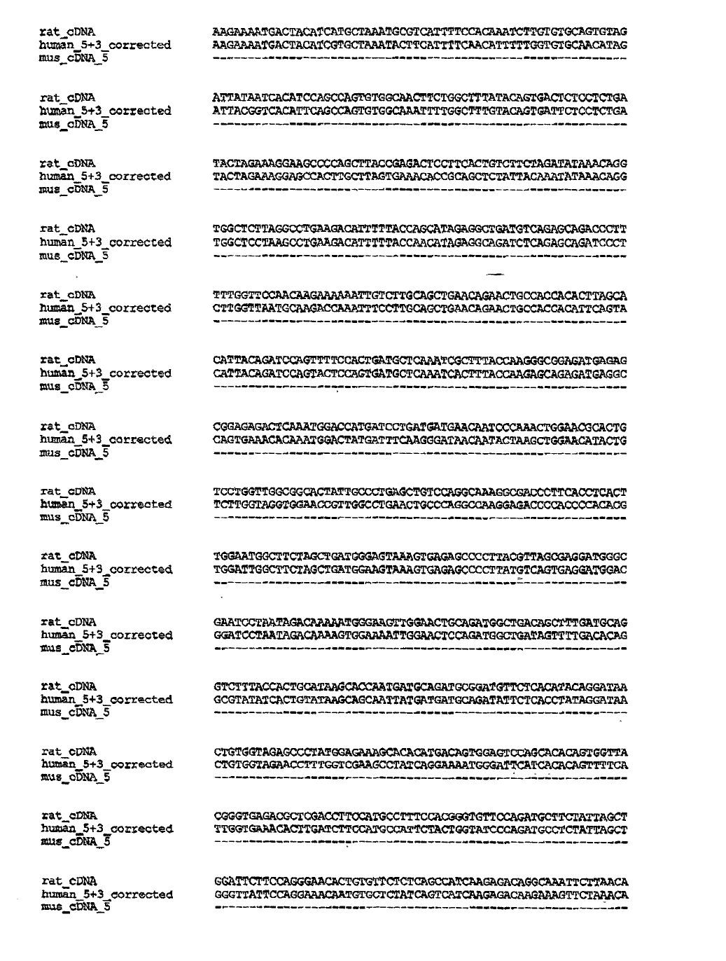

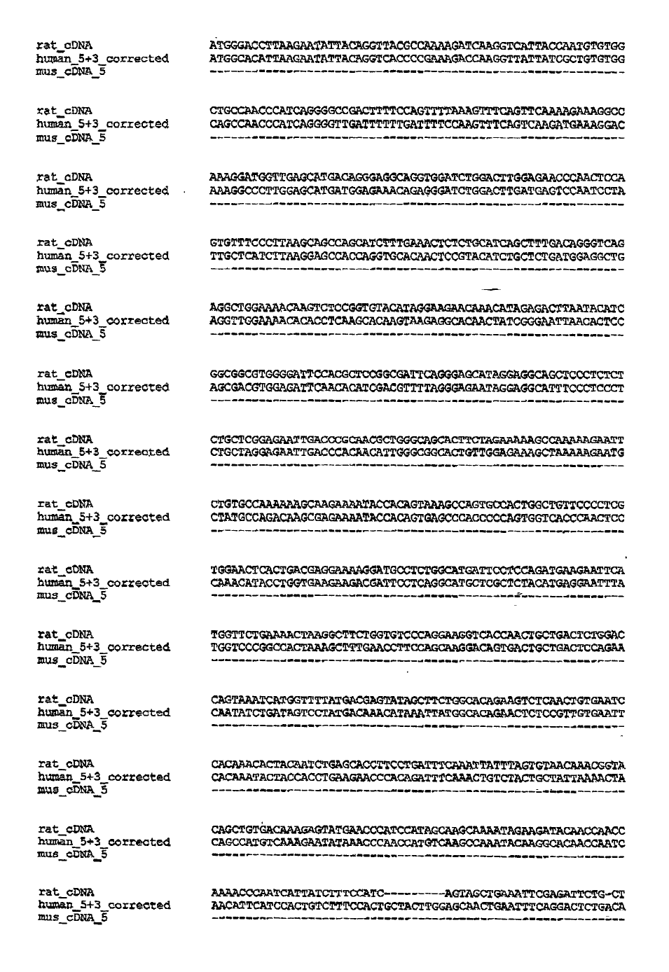

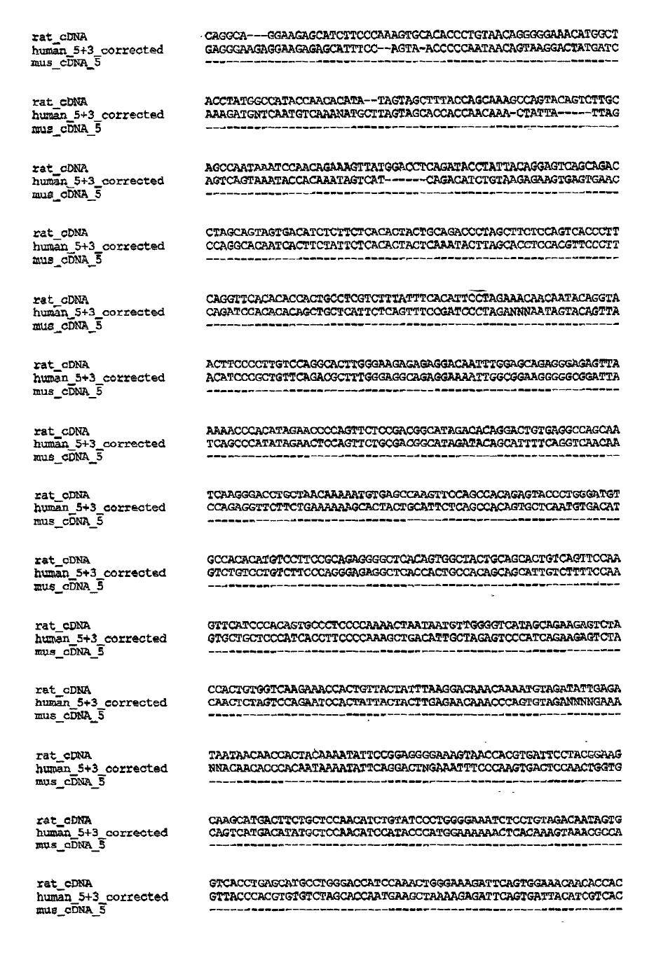

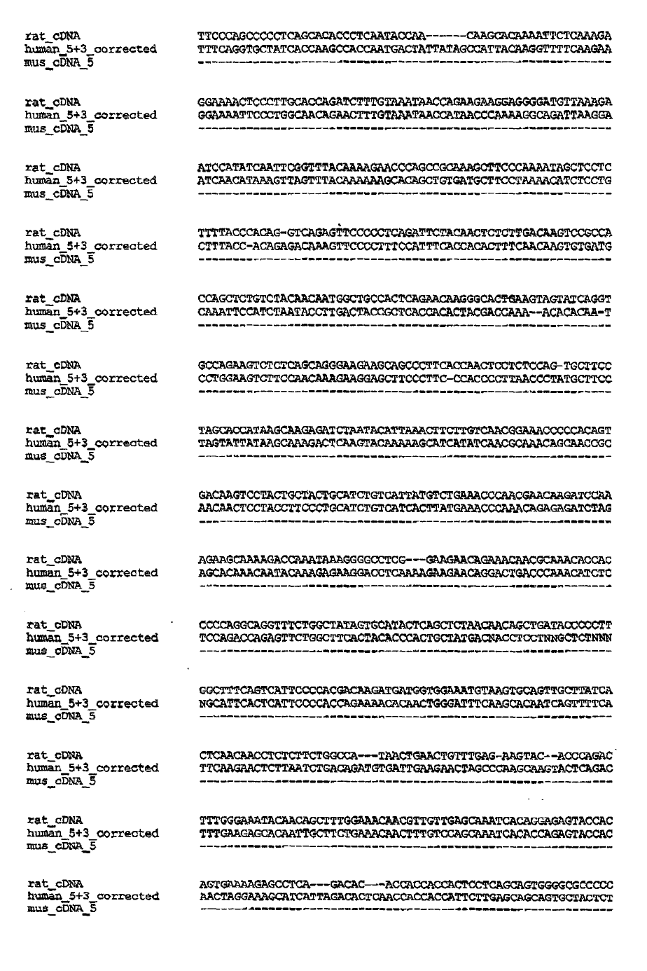

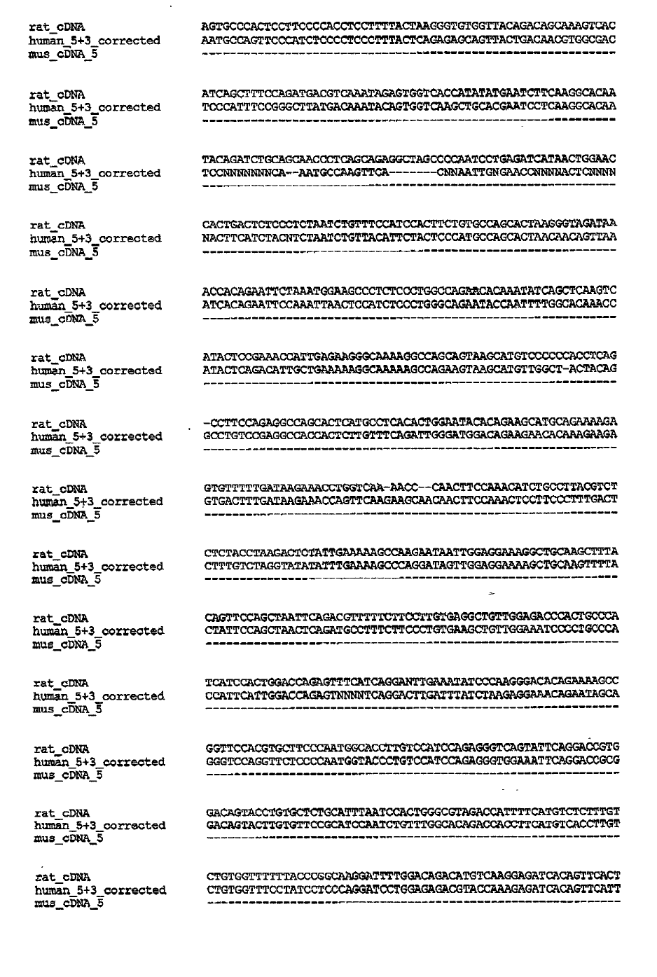

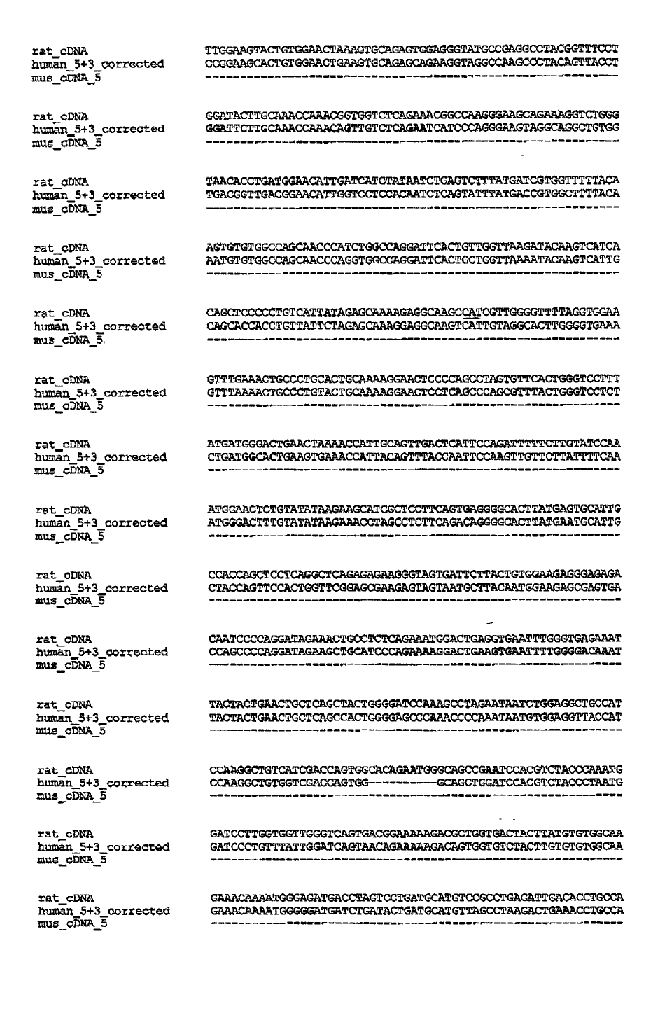

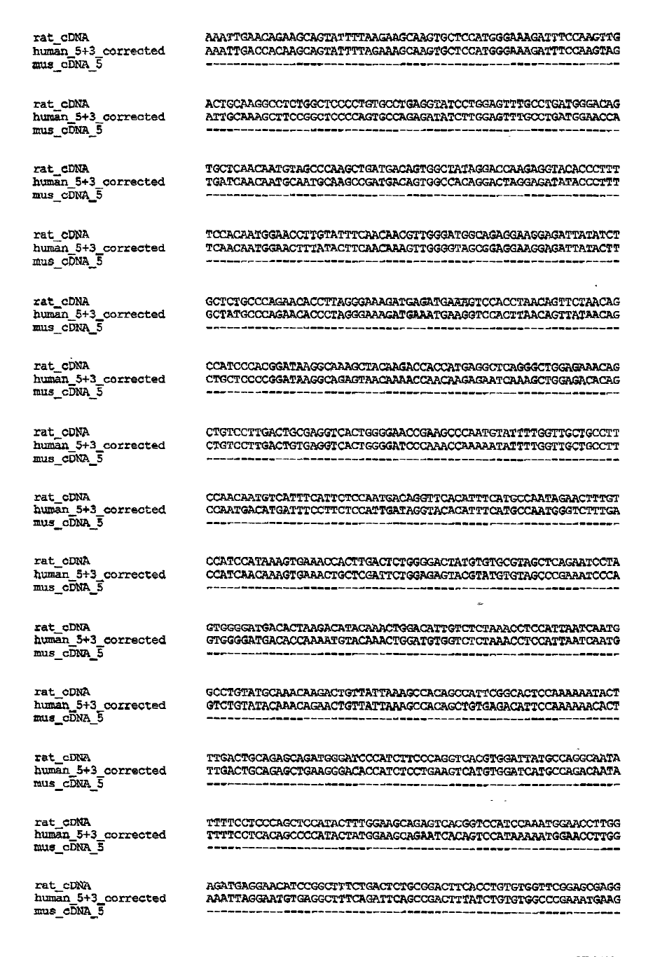

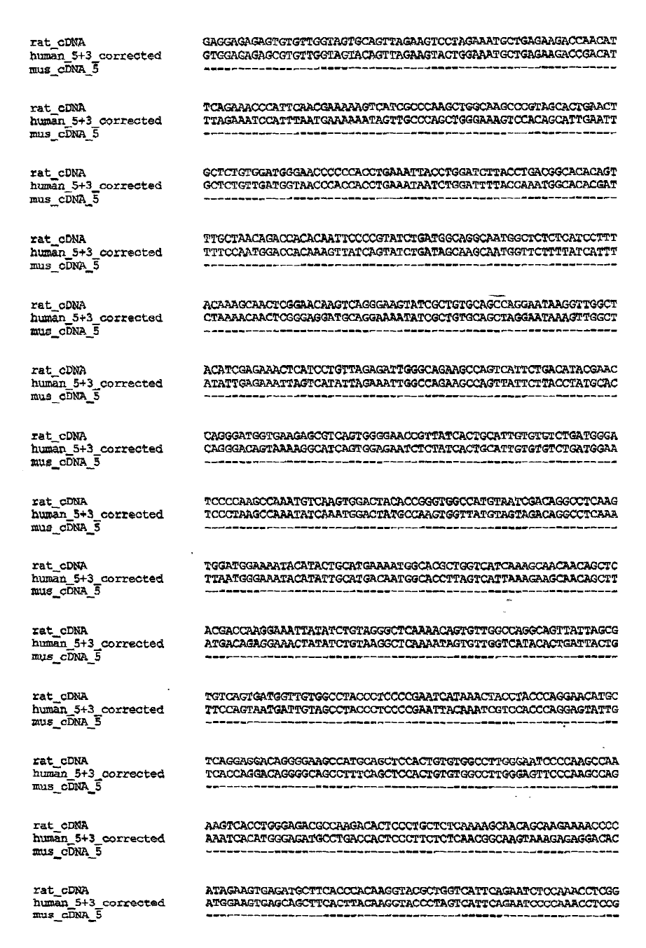

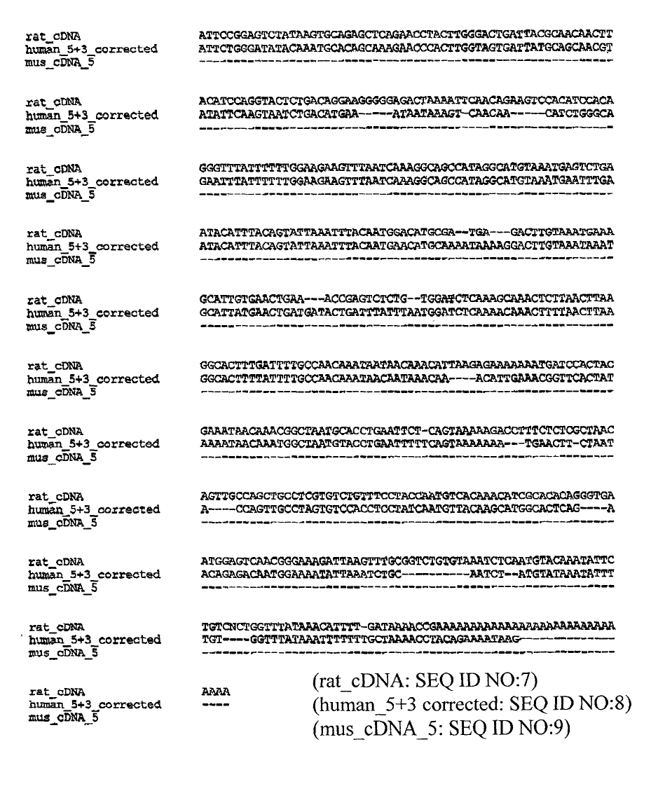

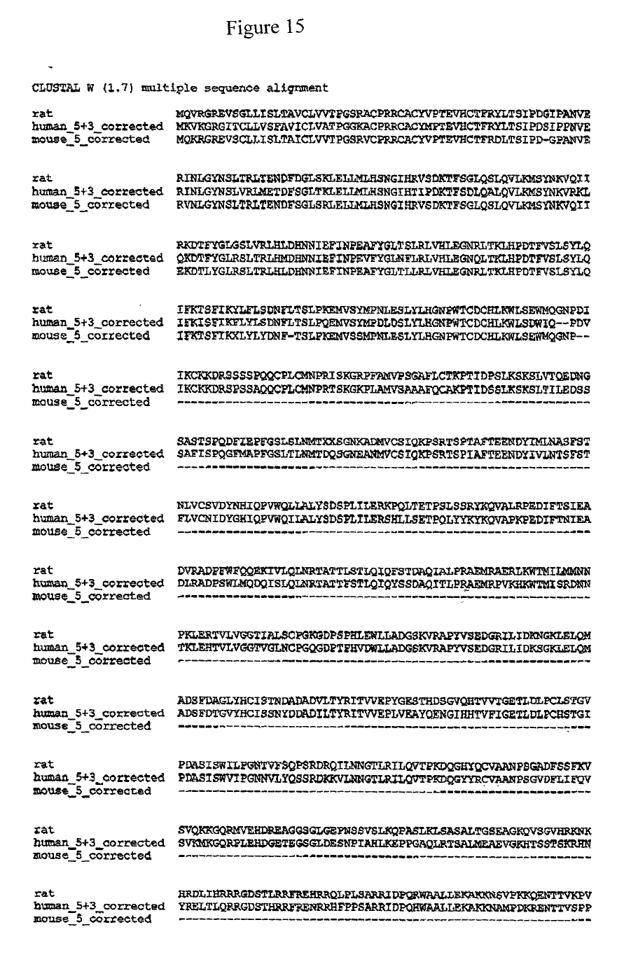

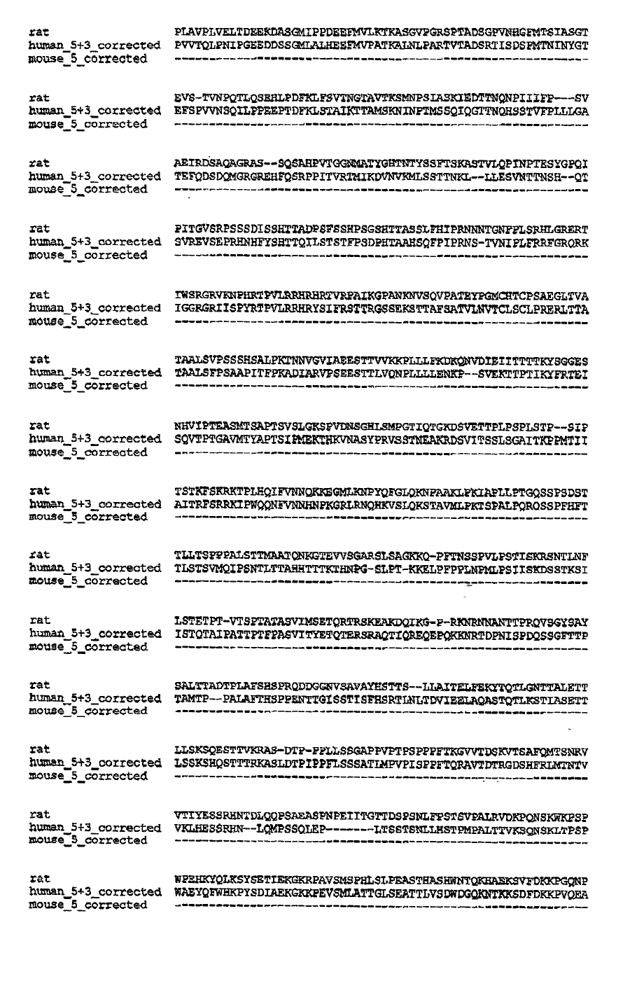

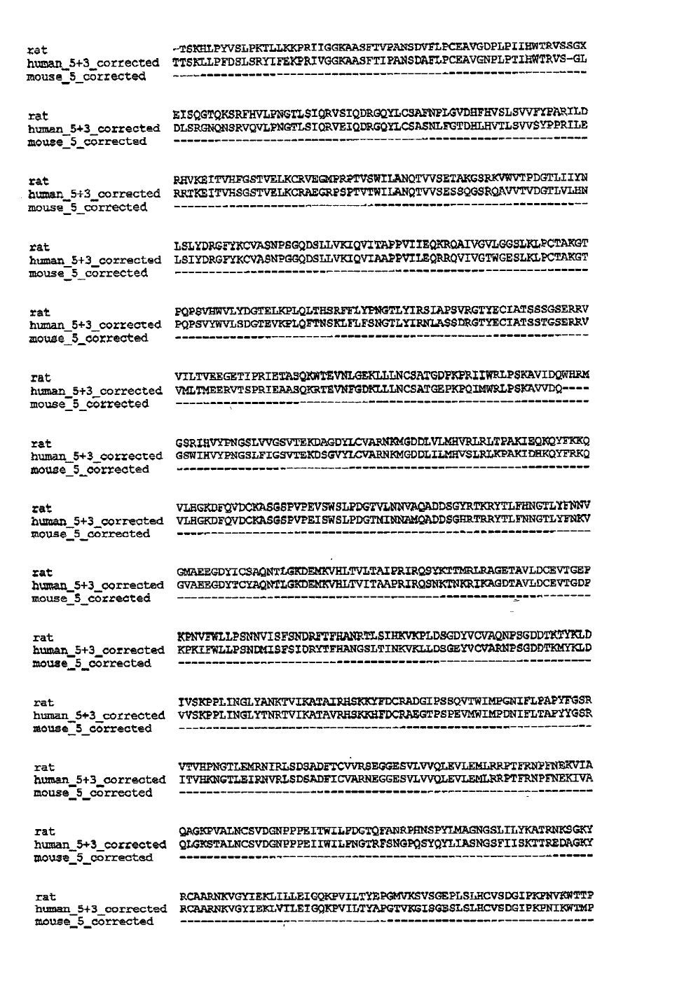

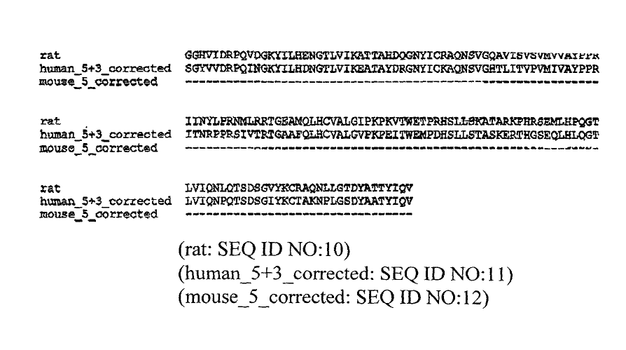

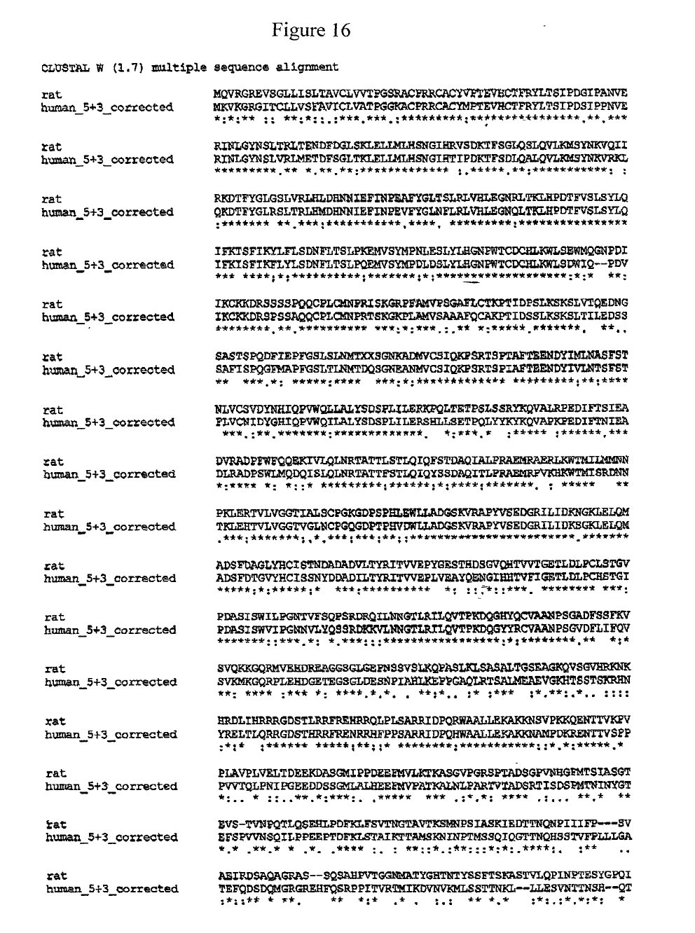

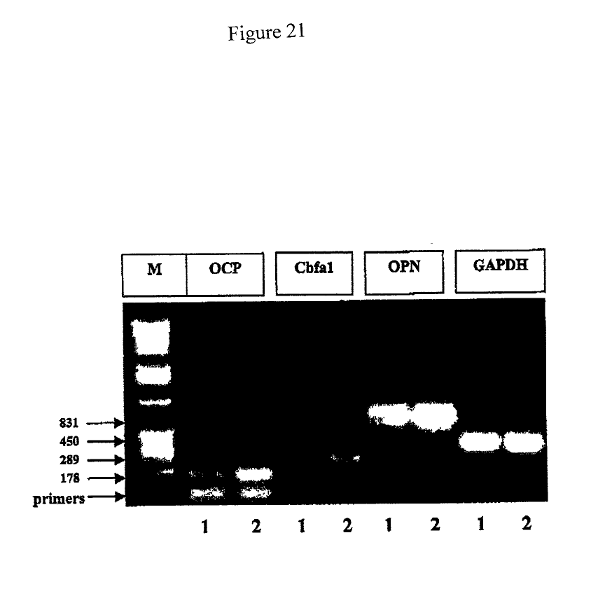

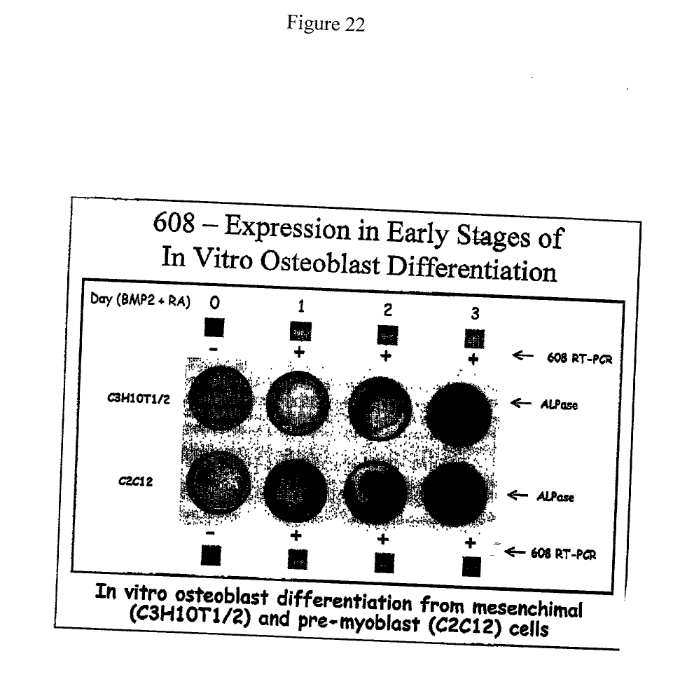

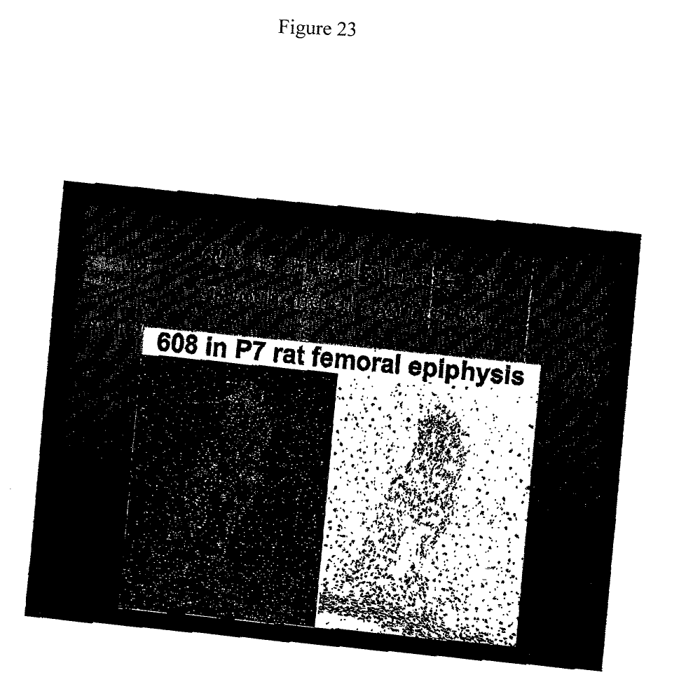

The deduced OCP protein was generated following the alignment (shown in FIGS. 14-16) of the rat, mouse and human cDNA sequences (FIGS. 1, 7 and 12, respectively) and the equivalent rat, mouse and human amino acid sequences (FIGS. 3, 21 and 22, respectively).

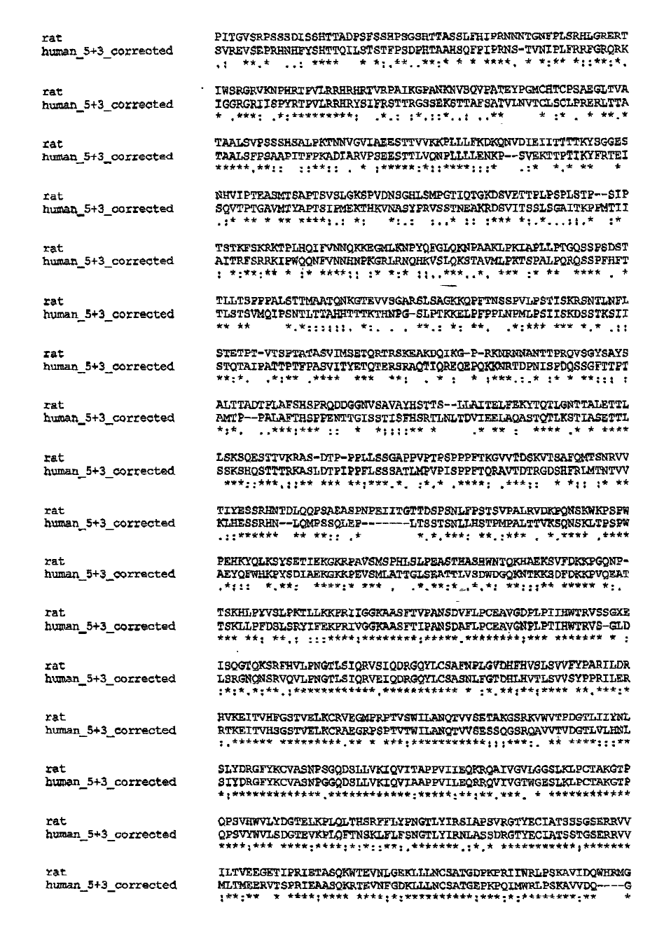

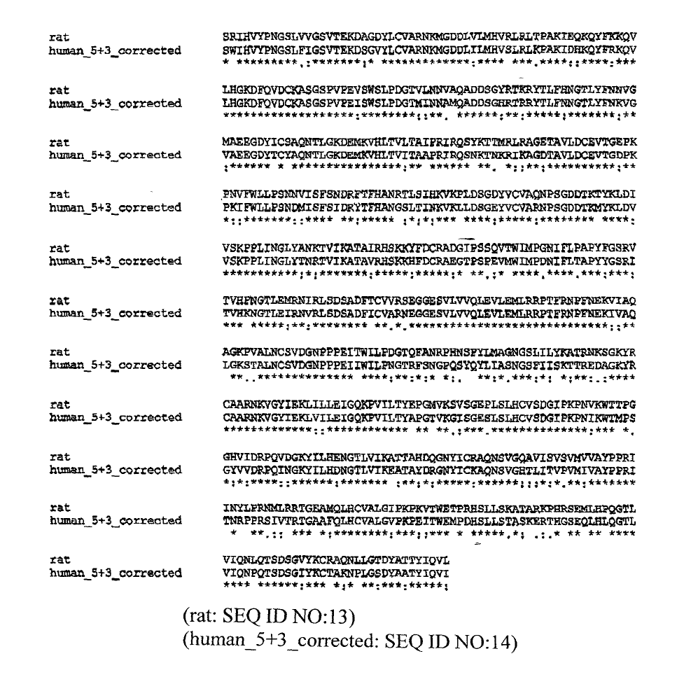

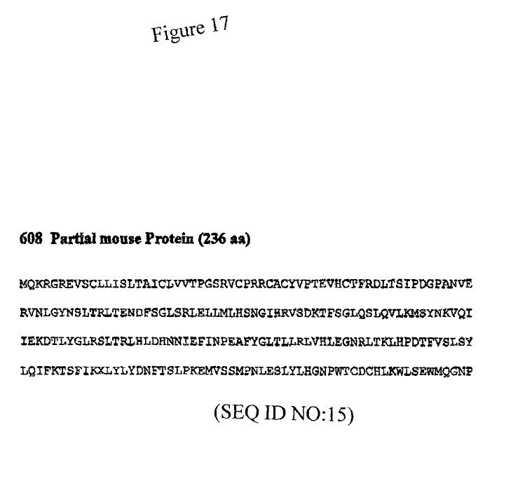

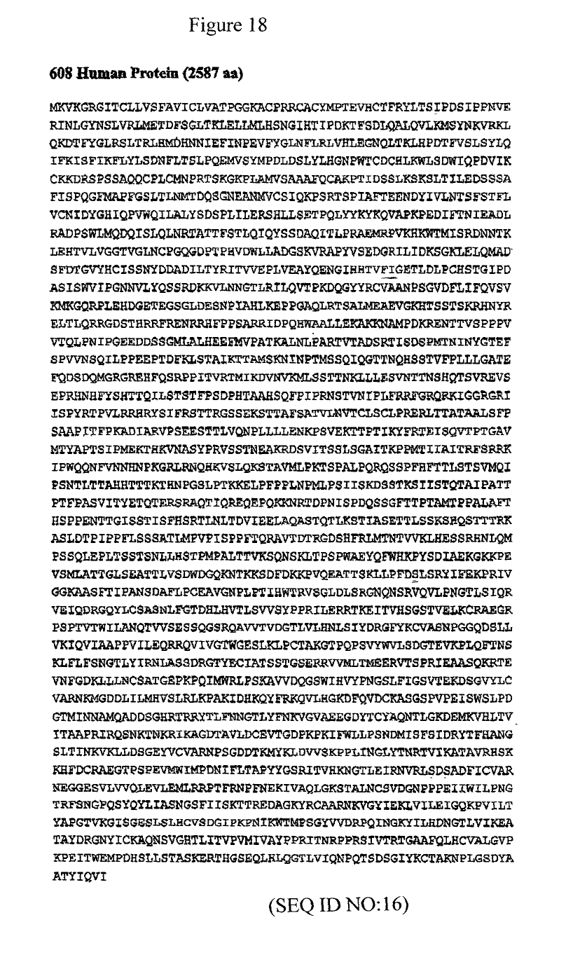

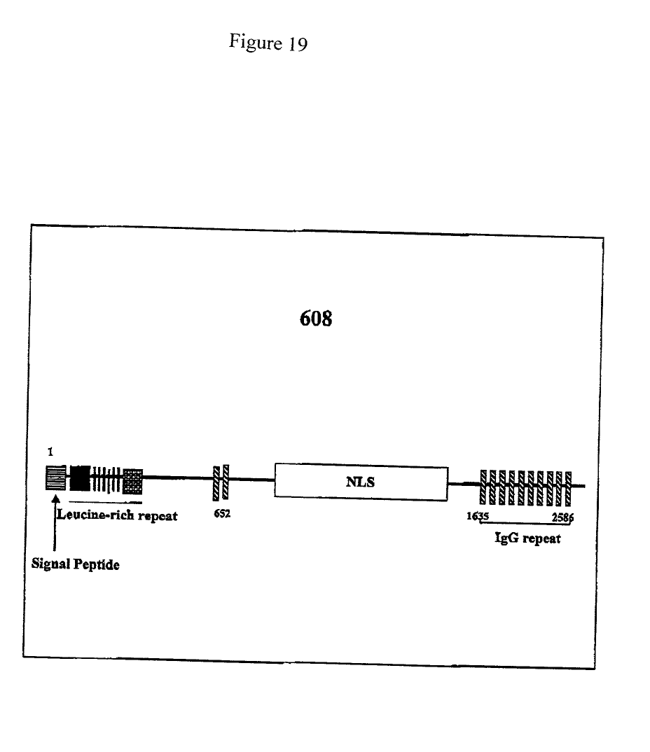

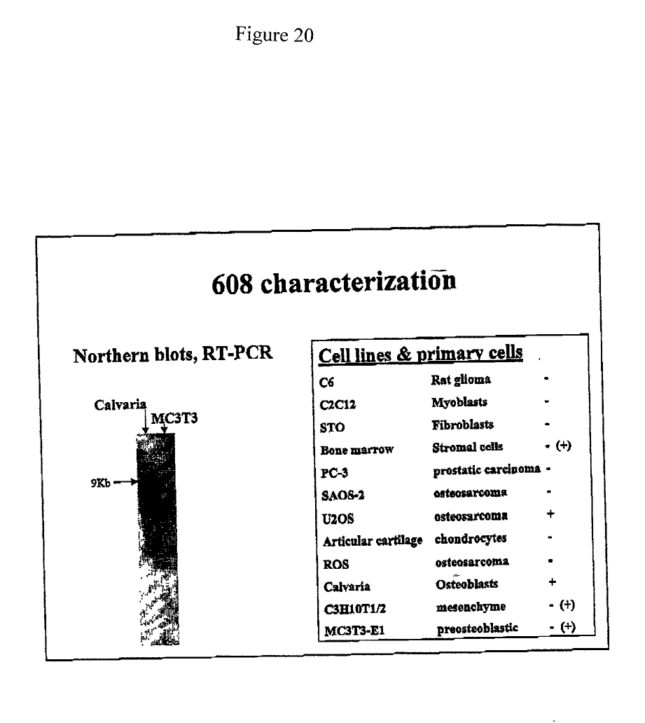

The deduced OCP protein contains the following features (as presented also in FIG. 18):

a. a cleavable, well-defined N-terminal signal peptide (aa 1-28);

b. a leucine-rich repeats region (aa 28-280). This region can be divided into N-terminal and C-terminal domains of leucine-rich repeats (aa 28-61 and 219-280, respectively). Between them, there are six leucine-rich repeats outliers (aa 74-96, 98-120, 122-144, 146-168, 178-200, 202-224). Leucine rich repeats are usually found in extracellular portions of a number of proteins with diverse functions. These repeats are thought to be involved in protein-protein interactions. Each leucine-rich repeat is composed of β-sheet and α-helix. Such units form elongated non-globular structures;

c. twelve immunoglobulin C-2 type repeats at amino acid positions 488-558, 586-652, 1635- 1704, 1732-1801, 1829-1898, 1928-1997, 2025-2100, 2128-2194, 2233-2294, 2324-2392, 2419-2487, 2515-2586. Thus, two Ig-like repeats are found immediately downstream to a leucine-rich region, while the remaining 10 repeats are clustered at the protein's C-terminus. Immunoglobulin C-2 type repeats are involved in protein - protein interaction and are usually found in extracellular protein portions;

d. no transmembrane domain.

e. Five nuclear localization domains (NLS) at positions: 724, 747, 1026, 1346 & 1618.

Overall, OCP probably belongs to the Ig superfamily. It is a serine rich protein (10.3% versus av. 6.3%), with a central nuclear prediction domain and an N terminal extracellular prediction domain.

### EXAMPLE 7

### Bone Fracture Healing

We have previously shown that expression of 608 RNA is bone-specific. Moreover, it seems to be specific to bone progenitors (as judged by their location in bone and involvement in normal bone modeling and remodeling processes - see our previous report) that do not yet express the known bone-specific markers. To further prove the relevance of 608-expressing cells to osteogenic lineage we have studied the patterns of 608 expression in the animal model of bone fracture healing that implies the activation of bone formation processes.

The sequence of physiological events following bone fracture is now relatively well understood. Healing takes place in three phases - inflammatory, reparative and remodeling. In each phase certain cells predominate and specific histological and biochemical events are observed. Although these phases are referred to separately, it is well known that events described in one phase persist into the next and events apparent in a subsequent phase begin before this particular phase predominates. These events have been described over the years in investigative reports and review articles (Ham, A. W. 1969. Repair of simple fracture, in Histology, sixth ed, Philadelphia, Lippincott, p 441 & Urist, M. R., Johnson, R. W. 1943. Calcification and ossification; healing of fractures in man under clinical conditions. J. Bone Joint Surg. 25: 375).

During the first phase immediately following fracture (the inflammatory phase), wide-spread vasodilatation and exudation of plasma lead to the acute edema visible in the region of a fresh fracture. Acute inflammatory cells migrate to the region, as do polymorphonuclear leukocytes and then macrophages. The cells that participate directly in fracture repair during the second phase (the reparative phase), are of mesenchymal origin and are pluripotent. These cells form collagen, cartilage and bone. Some cells are derived from the cambium layer of the periosteum and form the earliest bone. Endosteal cells also participate. However, the majority of cells directly taking part in fracture healing enter the fracture site with the granulation tissue that invades the region from surrounding vessels (Trueta, J. 1963. Role of vessels in osteogenesis. J. Bone Joint Surg. 45: 402). Note that the entire vascular bed of an extremity enlarges shortly after the fracture has occurred but the osteogenic response is limited largely to the zones surrounding the fracture itself (Wray, J. B. 1963. Vascular regeneration in healing fracture. Experimental study. Angiology 14: 134).

The invading cells produce tissue known as “callus” tissue (made up of fibrous tissue, cartilage, and young, immature fibrous bone), rapidly enveloping the ends of the bone, with a resulting gradual increase in stability of the fracture fragments. Cartilage thus formed will eventually be resorbed by a process that is indistinguishable except for its lack of organization from endochondral bone formation. Bone will be formed by those cells having an adequate oxygen supply and subjected to the relevant mechanical stimuli.

Early in the repair process, cartilage formation predominates and glycosaminoglycans are found in high concentrations. Later, bone formation is more obvious. As this phase of repair takes place, the bone ends graually become enveloped in a mass of callus containing increasing amounts of bone. In the middle of the reparative phase the remodeling phase begins, with resorption of portions of the callus and the laying down of trabecular bone along lines of stress. Finally, exercise increases the rate of bone repair (Heikkinen, E., Vihersaari, P., Penttinen, R. 1970. Effect of previous exercise on development of experimental fractures callus. Scand J. Clin. Lab. Invest. 25 (suppl 113): 32). In situ hybridization results have shown that OCP expression is confined to very specific regions where bone and cartilage formation is initiated.

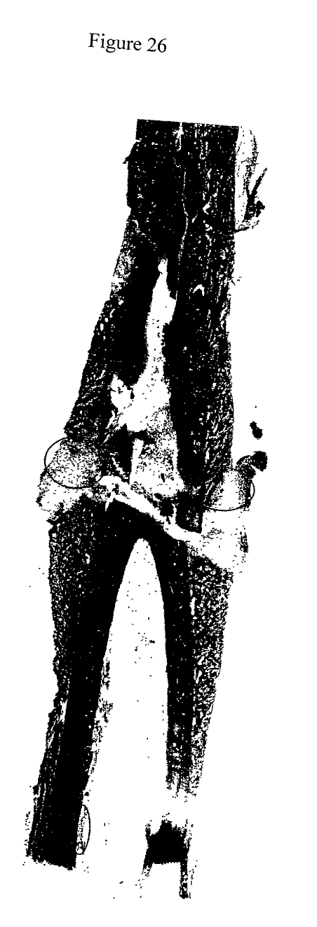

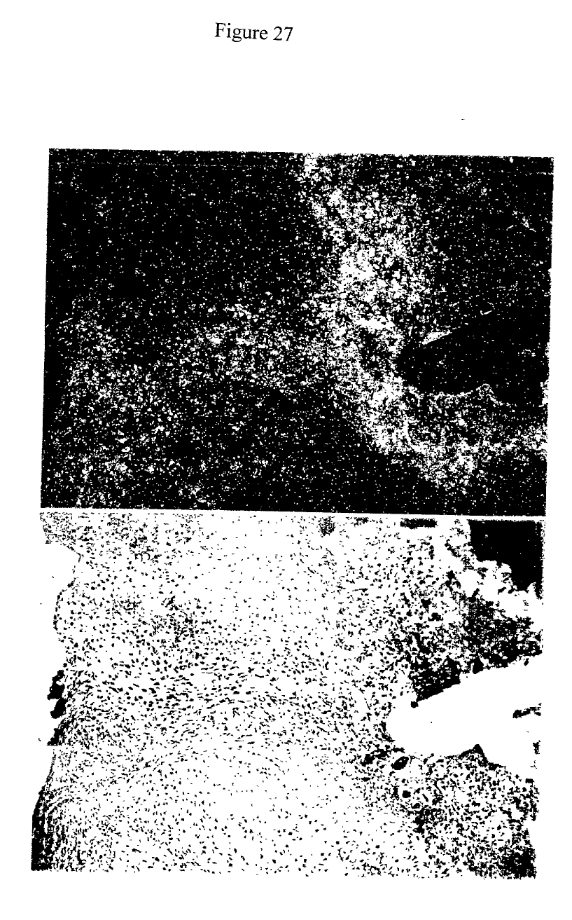

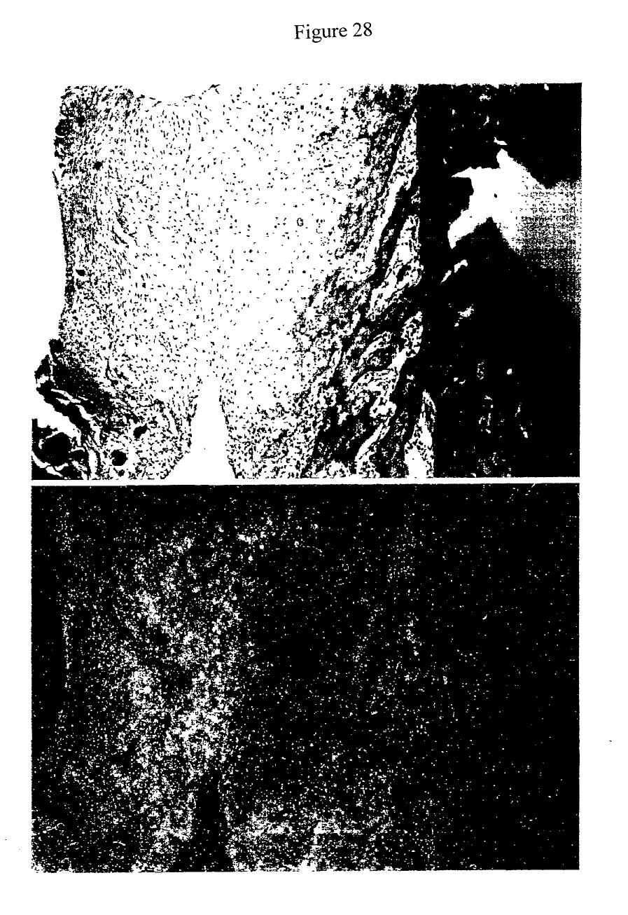

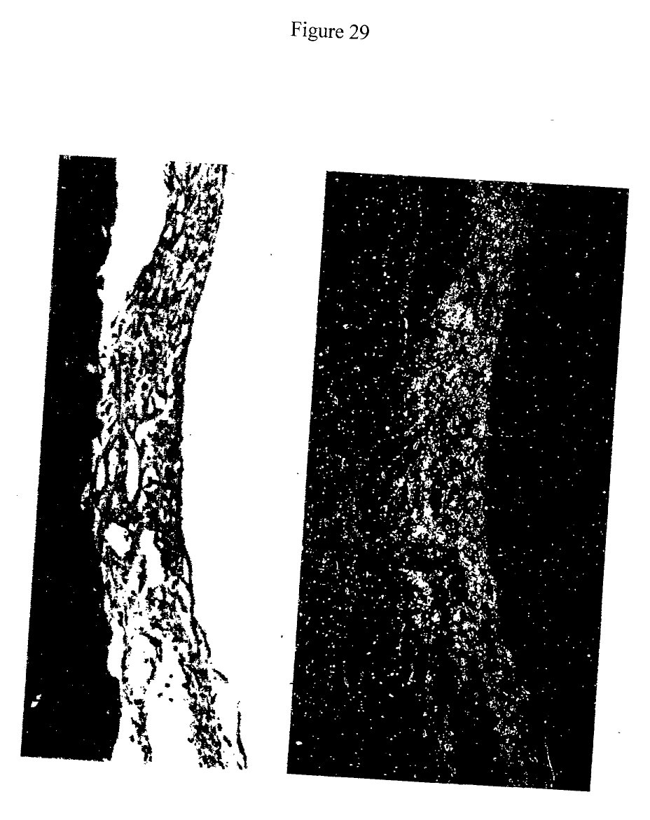

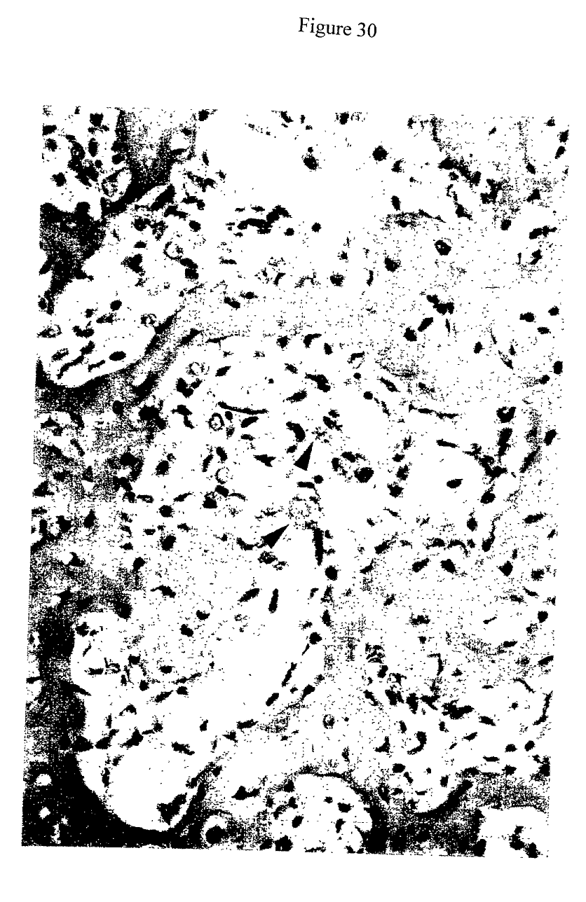

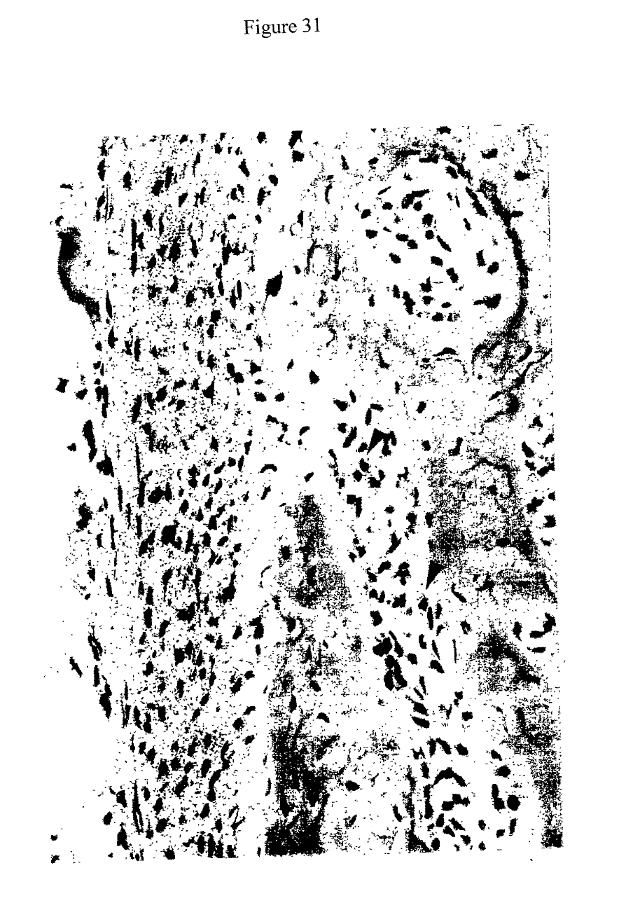

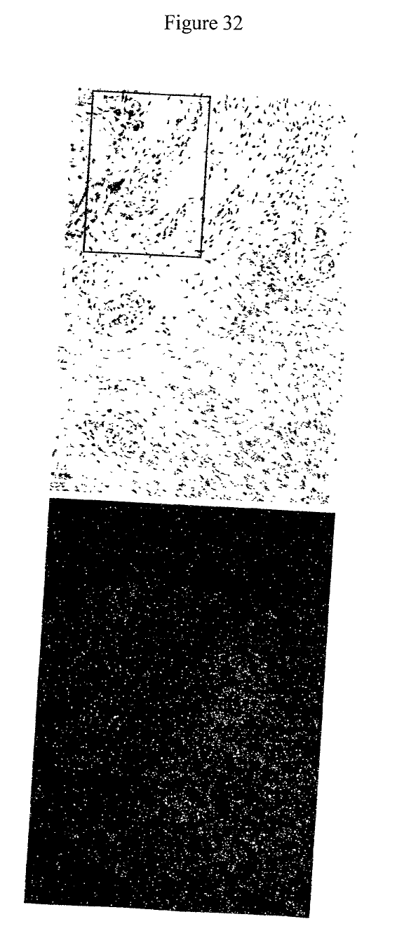

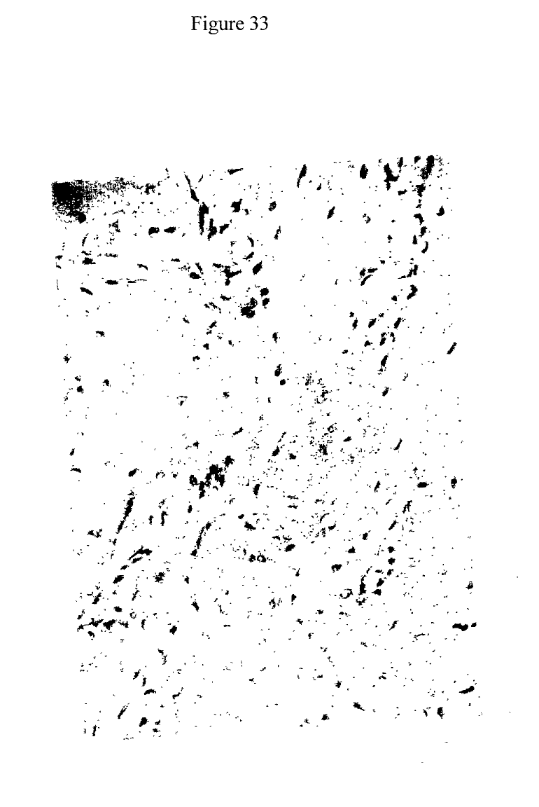

In order to find out if OCP expression is induced in an animal model of bone fracture healing, a standard midshaft fracture was created in rat femur by means of a blunt guillotine, driven by a dropped weight (Bonnarens et al. 1984. Orthop. Res. 2:97-101). 1, 2, 3 and 4 week-fractured bones were excised, fixed in buffered formalin, decalcified in EDTA solution and embedded in paraffin. All sections were hybridized with the OCP probe. The in-situ hybridization results show that a strong hybridization signal was apparent during the first and second weeks of fracture healing in the highly vascularized areas of the connective tissue within the callus (FIGS. 26-28), the endosteum (FIG. 29), the woven bone (FIG. 30) and the periosteum (FIG. 31). It may be noted that the periosteum is regarded as a source of undifferentiated progenitors participating in callus formation at the site of bone fracture. The hybridization signal disappeared slowly during further differentiation stages of fracture healing (three and four weeks) and was retained only in the vascularized connective tissue. FIG. 32 displays brightfield (left) and darkfield (right) microphotographs of a section of fractured bone healed for 4 weeks. In these later healing stages, the mature callus tissue was found to be comprised mainly by cancellous bone undergoing remodeling into compact bone, with little if any cartilage or woven bone present. The volume of the vascularized periosteal tissue is decreased but multiple cells in the periosteal tissue area of active remodeling of the cancellous bone covering the callus, show hybridization signal. This tissue covers the center of the callus and is also entrapped within the bone. (See FIGS. 32 and 33. The box in FIG. 32 is enlarged in FIG. 33). As in the earlier stages, no hybridization signal was found in chondrocytes and osteoblasts (FIGS. 27 and 33). Several OCP expressing cells are concentrated in the vascular tissue that fills the cavities resulting from osteoclast activity (marked by asterisks).

Fractures in the young heal rapidly, while adult bone fractures heal slowly. The cause is a slower recruitment of specific chondro-/osteo-progenitors for the reparative process. Denervation retards fracture healing by diminishing the stress across the fracture site, while mechanical stress increases the rate of repair probably by increasing the proliferation and differentiation of specific bone progenitor cells and as a result, accelerates the rate of bone formation. The above results confirm our conclusions (see also hereunder) that OCP is most probably involved in induction of cortical and trabecular bone formation and remodeling, endochondral bone growth during development, and bone repair processes. In addition, there is strong evidence that OCP expression is tightly regulated, and induced during the earliest stages of bone fracture repair when osteo-/chondro-progenitor cells are recruited. This observation suggests that OCP plays a role in this process.

Taking into account the pattern of 608 expression during the process of bone fracture healing, it is tempting to suggest that 608-positive precursor cells are involved not only in remodeling of intact bone but also in the repair processes of the fractured bone as well.

### EXAMPLE 8

### Transcriptional Regulation

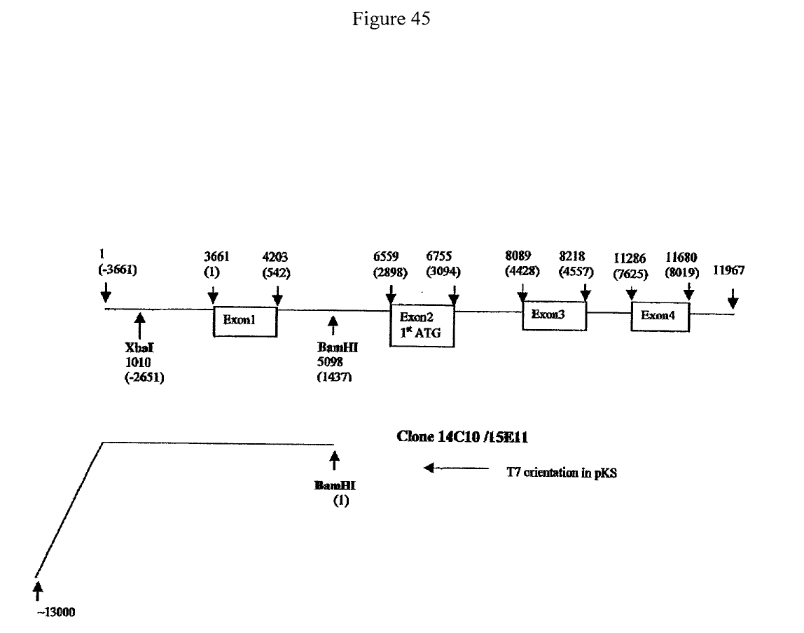

In order to clone the longest possible fragment which will contain the OCP regulatory region/s, bacs L4 and H7 were restricted with three different enzymes: BamHI, Bgl II and SauIIIA. The resulting fragments were cloned into the BamHI site of pKS. Ligation mixes were transformed into bacteria (E. coli - dH5□) and 1720 colonies were plated onto nitrocellulose filters which were screened with ³²P-labeled PCR fragment spanning the mouse-OCP-exon1. Positive colonies were isolated. Two identical clones, 14C10 and 15E11, contained the largest inserts (BamHI derived˜13Kb inserts). The structure of the insert compared to the “Lexicon” clone previously mentioned is illustrated in FIG. 45. The 14C 10 clone is longer than the OCP “Lexicon” clone by˜8Kb at the 5′ end.

### a) Cloning of Mouse OCP Promoter & UTR Upstream to the Reporter Gene - EGFP

The 1.4Kb genomic region of the mouse OCP gene, flanked by BamHI site (nuc 5098 of the “Lexicon” clone which is the start site of clone p14C10) and the first ATG codon (first nucleotide of exon 2), was synthesized by genomic PCR using the “Lexicon” clone as template and pre-designed primers: 5′ primer (For1) located upstream to the BamHI site (nucleotides 4587-4611 of the Lexicon clone) and 3′ primer (Rev 2) located immediately upstream to the first ATG (nucleotides 6560-6540 of the Lexicon clone) and tailed by a NotI site. The PCR product was cut by BamHI and NotI and the resulting 1.4Kb fragment was ligated to pMCSIE into BamHI/NotI sites upstream to the EGFP reporter gene. The resulting clone was designated pMCSIEm608prm1.4.

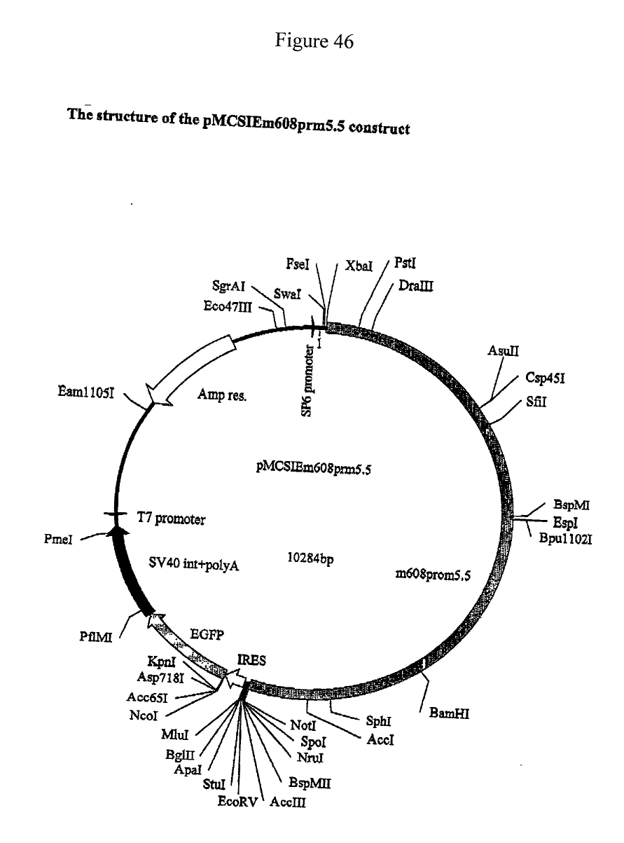

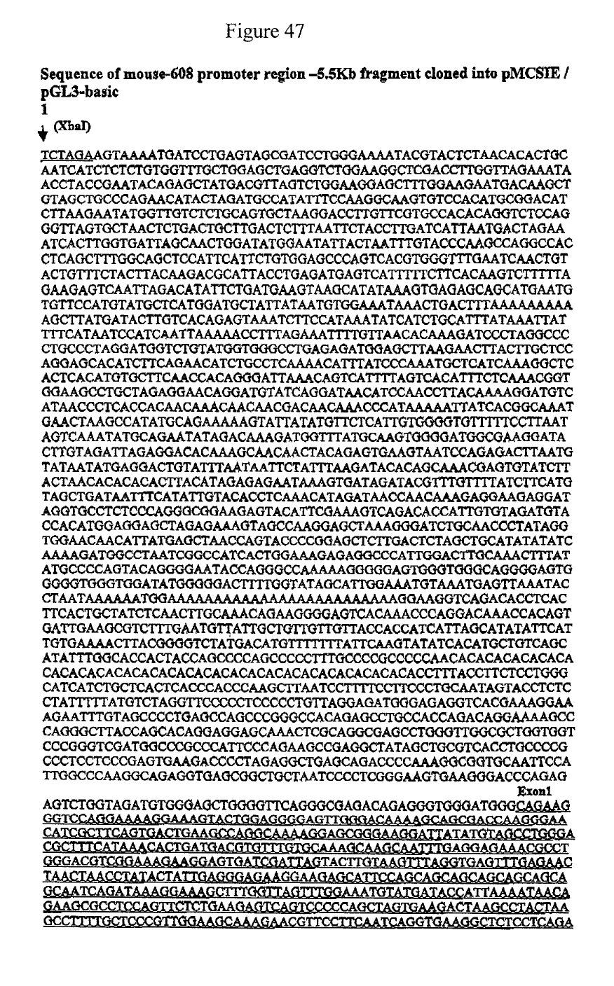

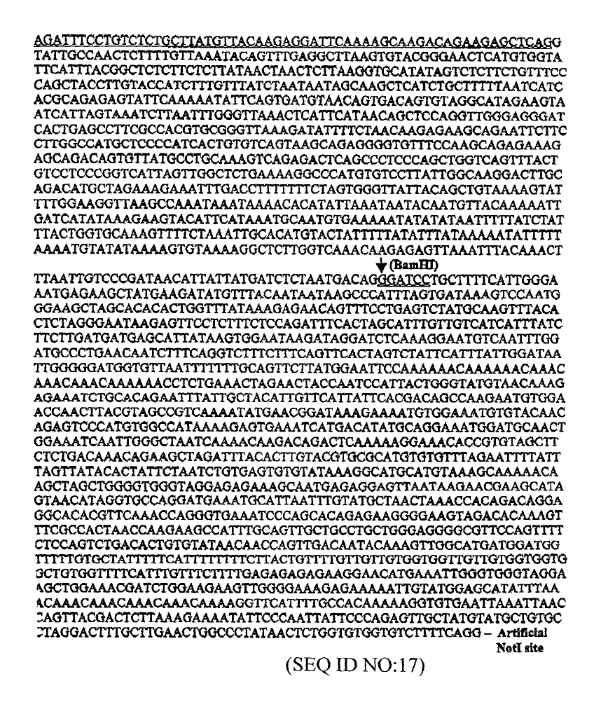

Clone p14C10 was cut by XbaI and BamHI and the excised 4.088Kb fragment was ligated into the BamHI and XbaI sites of pMCSIEm608prm1.4, upstream to the 1.4Kb insert. The resulting clone (shown in FIG. 46) was designated pMCSIEm608prm5.5 and contains 5552 nucleotides of the mouse 608 promoter and UTR upstream to EGFP. The insert of pMCSIEm608prm5.5 clone was completely sequenced, as may be seen in FIG. 47.

The whole 13Kb insert of p14C10 was excised by BamHI and ligated upstream to the 1.4Kb insert of of pMCSIEm608prm14 into the BamHI site. The resulting construct, pMCSIEm608prm14.5 contains a 14.5Kb fragment of the mouse-OCP promoter and UTR upstream to EGFP.

### b) Transient Transfection Results

The two constructs, pMCSIEm608prm5.5 and pMCSIEm608prm14.5, were injected to fertilized mouse eggs and two weeks old transgenic and control mice were sacrificed for the detection of GFP activity in calvaria and long bones. No specific fluorescence was detected, partly because of background fluorescence from various tissues and partly because of the cellular specificity of OCP expression. Therefore, the inventors decided to use the more sensitive luciferase gene as the reporter gene.

### c) Cloning of Mouse OCP Promoter & UTR Opstream to the Reporter Gene-Luciferase

Both inserts of pMCSIEm608prm5.5 and of pMCSIEm608prm14.5 were also cloned upstream to luciferase, in Promega's pGL3-Basic vector. The 5.5Kb insert of pMCSIEm608prm5.5 was excised by EcoRV and XbaI and ligated to SmaI and NheI sites of pGL3-Basic vector. The resulting clone is designated pGL3basicm608prm5.5.

Plasmid pMCSIEm608prm14.5 was restricted by NotI and the cohesive ends of the linearized plasmid were filled and turned into blunt ends. The 14.5Kb insert was then excised by cutting the linear plasmid by SalI. The purified 14.5Kb fragment was ligated to the XhoI and HindIIl (filled in) sites of pGL3-basic upstream to the luciferase gene to create the construct designated pGL3basicm608prm14.5.

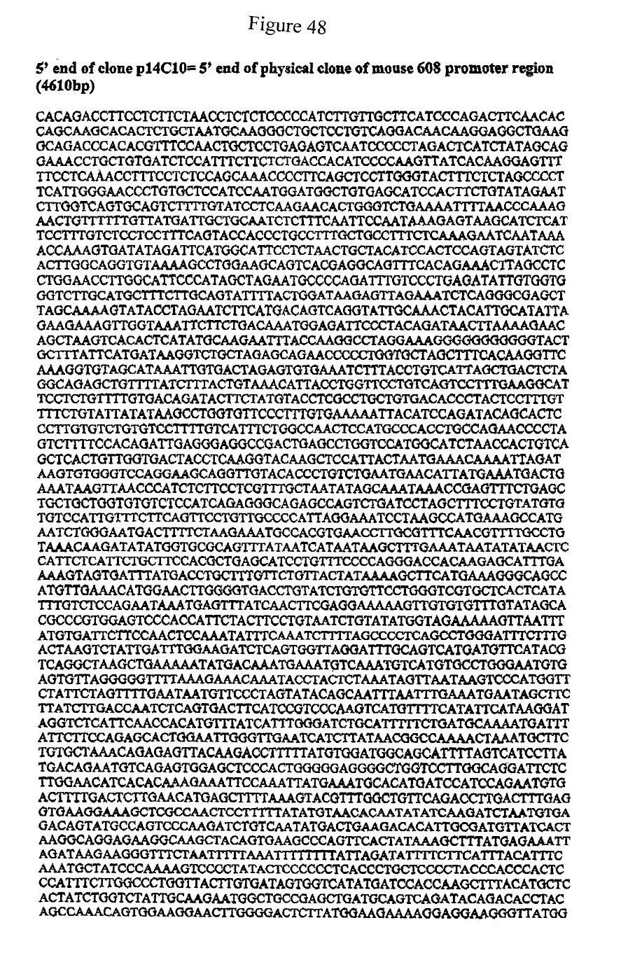

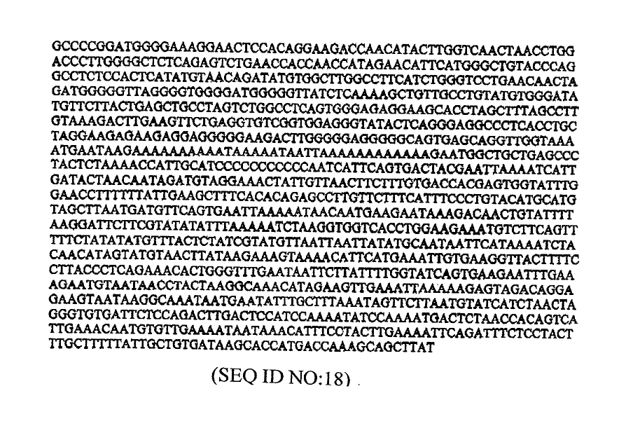

Sequence analysis of the 5′ end of the 13Kb insert of clone p14C10 is in process. Currently, 4610 bp have been sequenced at this end (FIG. 48).

### d) Transient Transfection Results

At this stage transient transfection of both constructs to primary calvaria cells, resulted in 10-fold expression only upon pMCSIEm608prm14.5 transfection. No enhanced promoter activity was observed upon pMCSIEm608prm5.5 transfection. These observations suggest that the region between the 5′ end of pMCSIEm608prm14.5 and the 5′ end of pMCSIEm608prm5.5 is necessary for full promoter activity. Further analysis is in process to detect all the sequences that are necessary and sufficient for maximal promoter activity and tissue specific OCP induction or repression in various cell systems.

### e) Analysis of TF (Transcription Factor) Binding DNA Rlements Common to Mouse and Human OCP

The inventors searched for known DNA binding elements of similarity upstream of the human and mouse OCP ATG using the DiAlign program of Genomatix GmbH. The genomic pieces used are the proprietary mouse genomic OCP and reverse complement of AC024886 92001 to 111090. The locations of the ATG in these DNA pieces are:

575 on rat CDNA

6521 on mouse genomic

3381 on the piece extracted from human genomic DNA AC0024886

14 elements were extracted in this procedure and analyzed for transcription binding motifs using the MatInspector.

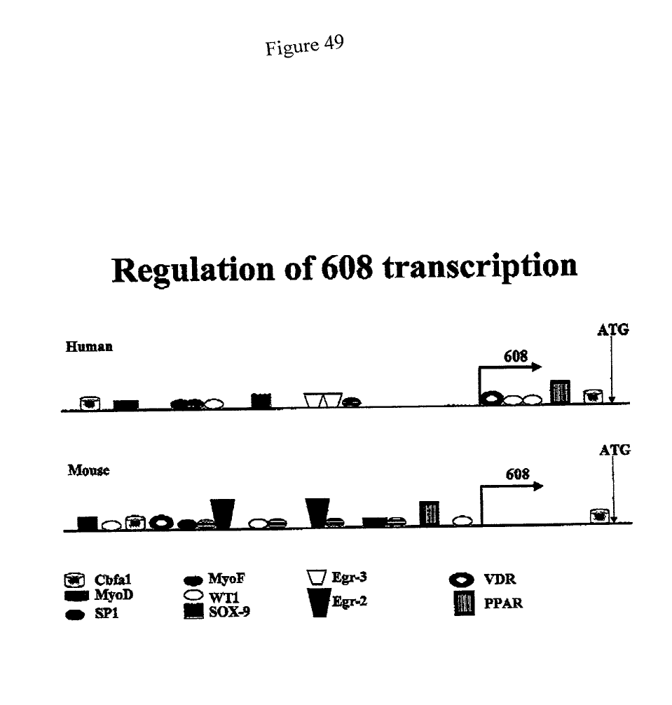

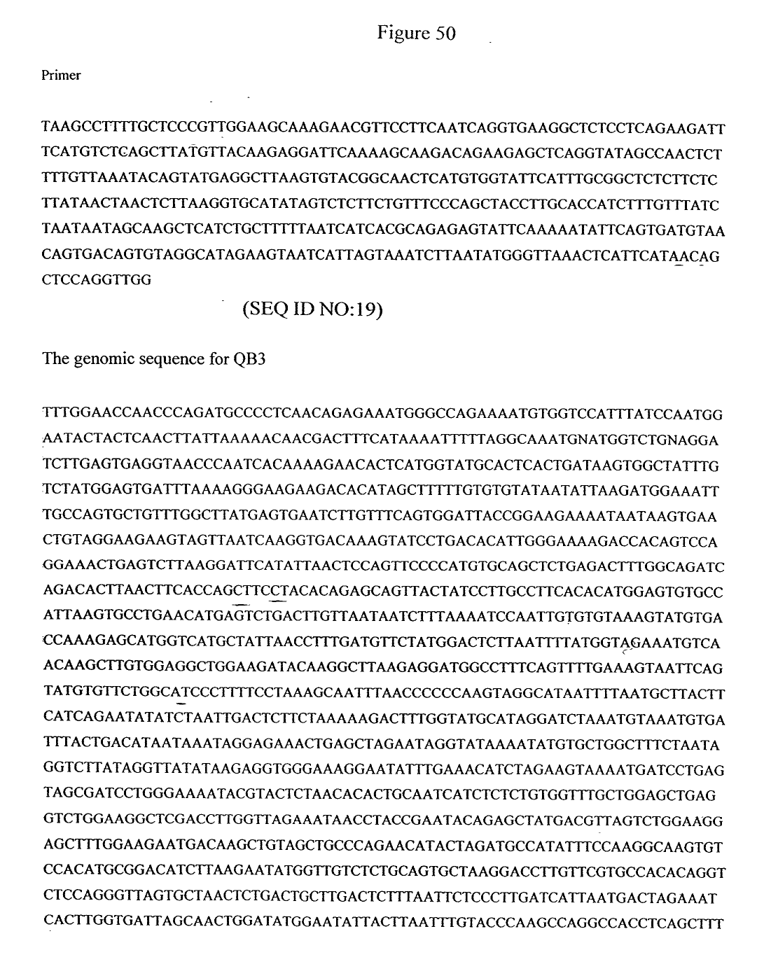

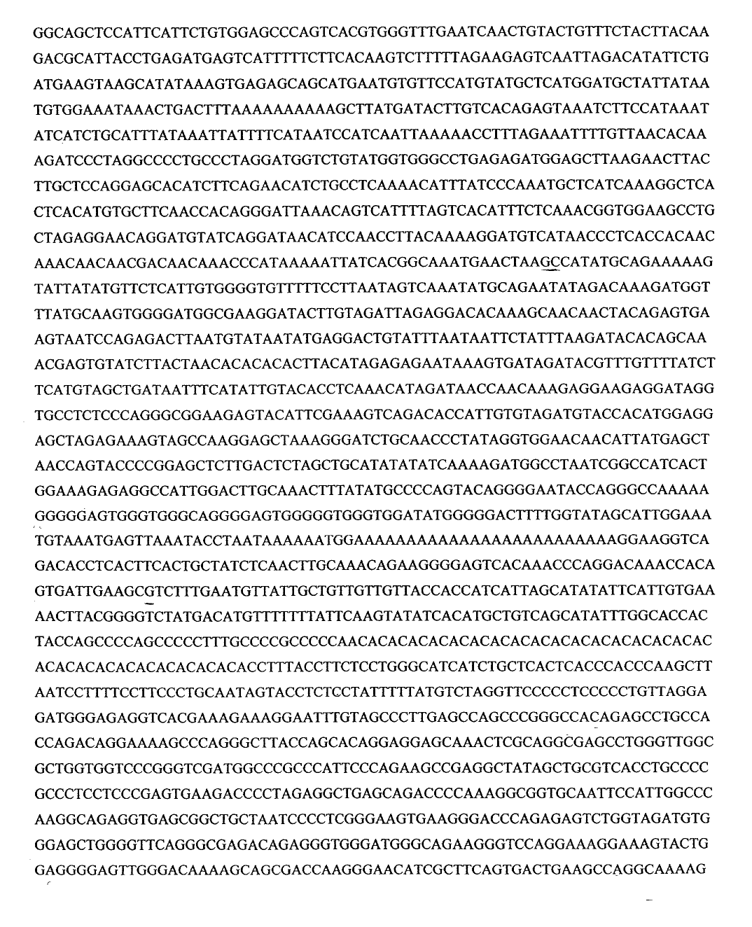

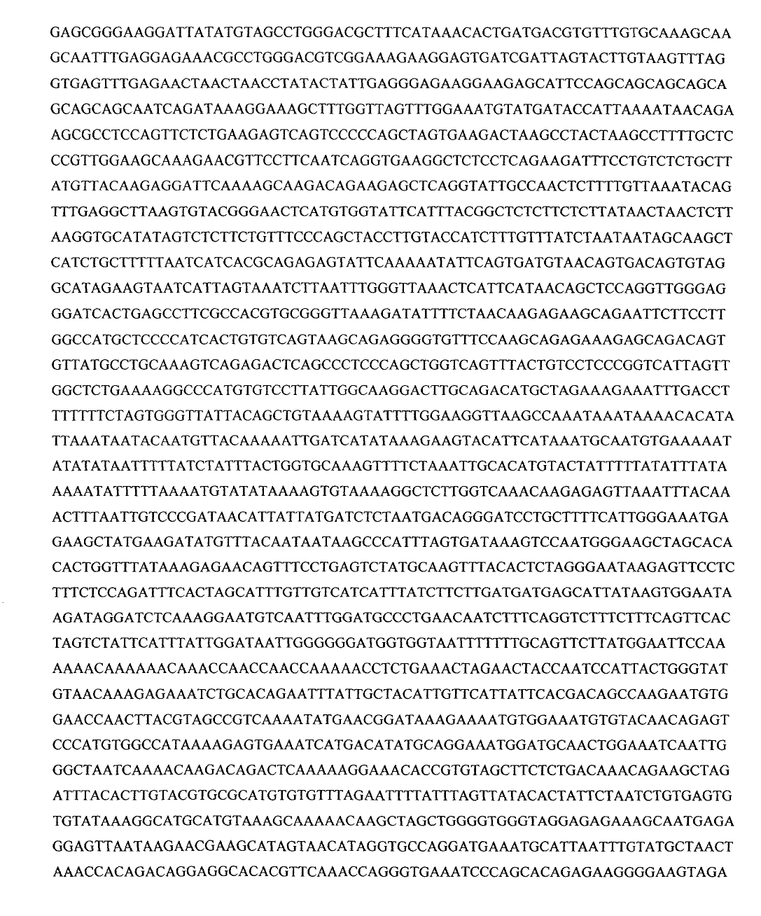

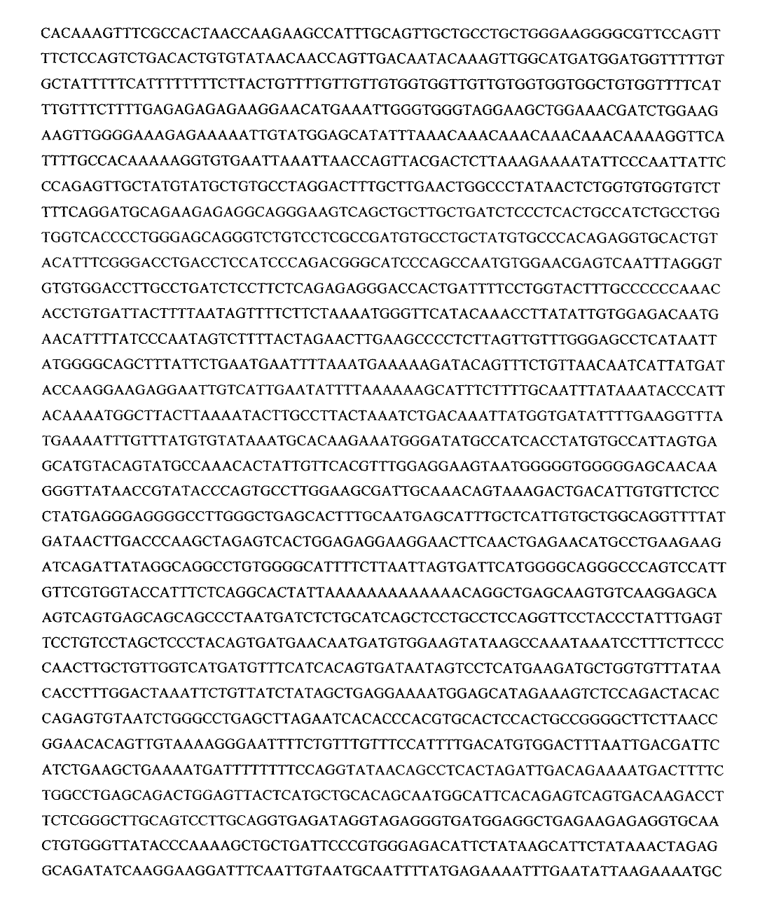

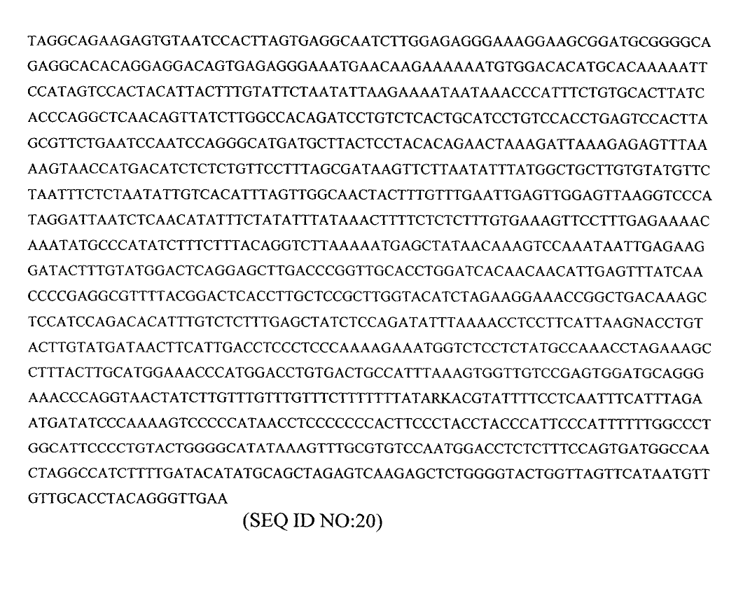

Some of the main “master gene” binding sites are illustrated in FIG. 49. Among them are the osteoblast-/chondrocyte-specific Cbfal factor, the chondrocyte-specific SOX 9 factor, the myoblast-specific Myo-D and Myo-F factors, the brain- and bone-specific WT1, Egr 3 and Egr 2 factors (Egr superfamily), the vitamin D-responsive (VDR) factor, the adipocyte-specific PPAR factor and the ubiquitous activator SP1.

### EXAMPLE 9

### Expression Pattern and Regulation of Gene 608

Expression of gene 608 in regard to other osteogenic lineage markers: Expression of gene 608 was tested in primary cells and in cell lines with regard to expression of various markers of osteogenic and chondrogenic lineages. The results of this analysis are summarized in Table 2.

TABLE 2

| | | | | Alkaline | | | |
|---|---|---|---|---|---|---|---|
| Cells | 608 | Collagen I | Collagen II | phosphatase | Osteocalcin | Cbfal | Osteopontin |
| STO (fibroblasts) | − | − | + | − | + | + | + |
| ROS | − | − | − | + | + | +/− | + |
| (osteosarcoma) | | | | | | | |
| MC3T3 (pre- | + | − | − | + | + | + | + |
| osteoblasts) | | | | | | | |
| C2C12 (pre- | − | − | − | − | + | − | + |
| myoblasts) | | | | | | | |
| C6 (glioma) | − | | | − | | | |
| Calvaria mouse | + | | | + | | | |
| Calvaria rat | + | | | + | | | |
| C3H10T1/2 | − | − | + | − | + | − | + |
| (mesenchymal | | | | | | | |
| stem cells) | | | | | | | |

Expression of 608 is restricted to committed early osteoprogenitor cells.

Expression of 608 is restricted to committed early osteoprogenitor cells.

### EXAMPLE 10

### OCP Expression is Mechanically Induced in MC3T3 E1 Cells

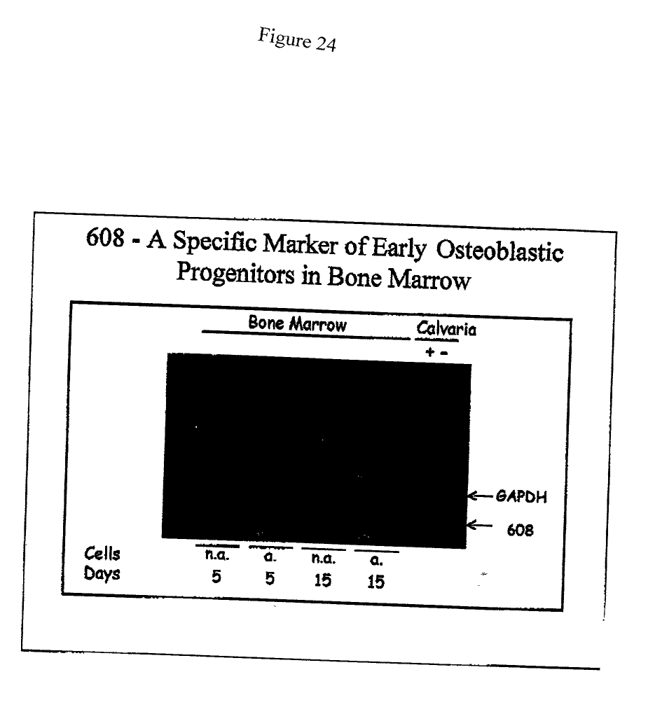

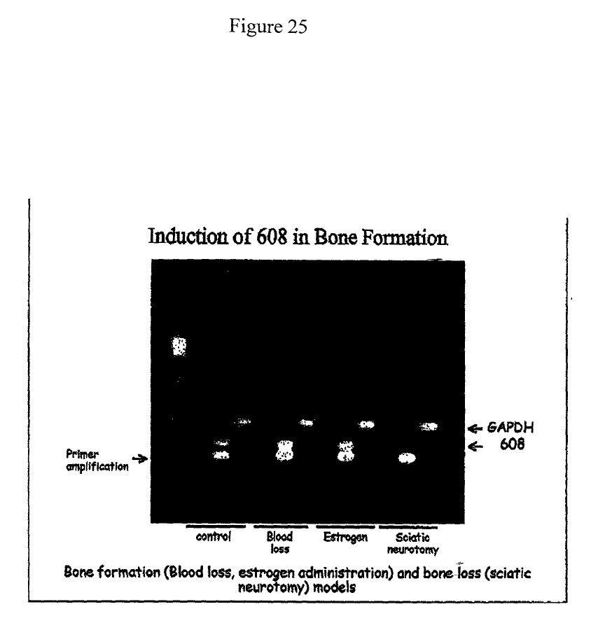

OCP transcription was detected by RT-PCR in mouse calvaria cells, U2OS cells (human osteosarcoma cell line), and human embryonal bone (FIG. 24). OCP was initially discovered as being upregulated during mechanical stress in calvaria cells. In the present invention, we demonstrate that the influence of mechanical stress on OCP expression can be reproduced in another cell system using a different type of mechanical stimulation. In serum-deprived MC3T3-E1 pre-osteoblastic cells, mechanical stimulation caused by mild (287x g) centrifugation markedly induced OCP mRNA accumulation (FIG. 25). We have also noticed that other osteoblastic marker genes (osteopontin, ALP (staining -not shown)& Cbfal) were transcriptionally augmented by this procedure (FIG. 25). The RT-PCR product of a non-osteoblastic marker gene (GAP-DH) was used as a control to compare RNA levels between samples. No increased expression was noticed when the latter primers were used. It may be noted that no expression was detected in non-osteoblastic cells (FIG. 24), suggesting that OCP expression is specifically induced in osteogenesis.

### EXAMPLE 11

### OCP Induction During Endochondral Growth - In Situ Hybridization Analysis

Our previous results demonstrated that OCP is expressed during adult mice bone modeling and remodeling. The expression was restricted to the following regions:

1 perichondrium

2 periosteum

3 active remodeling and modeling regions

4 perivascular connective tissue

5 articular cartilage covering cells

6 embryo-condensed mesenchymal cells—head, vertebrae & trunk

7 ectopic bone formation

No previous observations suggest any role for OCP in bone development or initiation of endochondral ossification (longitudinal growth of long bones). Thus, the inventors decided to study the expression pattern of OCP by in situ hybridization on sections of bones from 1 week old mice. At this stage of bone development, osteogenesis starts within the epiphysis (secondary ossification center). The hind limb skeleton of 1 week old rat pups (femur together with tibia) was fixed in buffered formalin and longitudinal sections of decalcified tissue were processed for in situ hybridization according to standard in-house protocol. Autoradiographs were developed, stained with hematoxylin-eosin and studied under microscope using brightfield and darkfield illumination.

A strong fluorescence signal was observed all over the second ossification center using OCP probes (FIG. 27). In addition, the hybridization signal delineates periosteal and perichondrial tissue in a way similar to that found earlier in adult bones. Surrounding mature chondrocytes displayed no signal. A very faint signal was observed using the osteocalcin probe which is a marker of mature osteoblasts (not shown).

We can conclude that OCP is expressed in osteoprogenitor cells that initiate endochondral ossification during bone development.

### EXAMPLE 12

### In Vivo Regulation by Stimuli Either Promoting or Suppressing Bone Formation: Estrogen Administration, Blood Loss and Sciatic Neurotomy

Osteogenic cells are believed to derive from precursor cells present in the marrow stroma and along the bone surface. Blood loss, a condition that stimulates hemopoietic stem cells, activates osteoprogenitor cells in the bone marrow and initiates a systemic osteogenic response. High-dose estrogen administration also increases de novo medullary bone formation possibly via stimulation of generation of osteoblasts from bone marrow osteoprogenitor cells. In contrast, skeletal unweighting, whether due to space-flight, prolonged bed-rest, paralysis or cast immobilization leads to bone loss in humans and laboratory animal models. To detect alteration in OCP expression pattern following the above procedures, the following experiments were performed on two month old mice:

estrogen administration (500 μg/animal/week),

bleeding (withdrawing approximately 1.6% body weight),

unilateral (right limb) sciatic neurotomy,

control groups for each treatment

Total RNA was extracted from long bones after two-day treatment and RT-PCR using OCP-specific primers was performed. The results demonstrate that OCP expression was highly enhanced following blood loss and estrogen administration, while down-regulation was observed following sciatic neurotomy (FIG. 29).

By having a unique cell marker (OCP) we can show that the above procedures induce or reduce bone formation by increasing ordecreasing the number of osteoprogenitor cells. The above results suggest once more that OCP is a major member of a group of “bone specific genes” that regulate the accumulation of bone specific precursor cells.

### EXAMPLE 13

### OCP Induction During Osteoblastic Differentiation of Bone Marrow Stroma Cells

Bone formation should be augmented in trabecular bone and cortical bone in osteoporotic patients. We have previously detected OCP expression in periosteum and endosteum (surrounding the cortical bone) but no signal was apparent in bone marrow cells. The latter cells normally differentiate to mature osteoblasts embedded in the trabecular and cortical bone matrix.

To further assess OCP expression in bone marrow progenitor cells, the inventors extracted total RNA from mouse and rat bone marrow immediately after obtaining it and after cultivation for up to 15 days in culture. No OCP-specific RT-PCR product was detected with RNA from freshly obtained bone marrow (both in adherent and non-adherent) cells. However, a faint signal was found after 5 days in culture, and it was further enhanced when RNA from cells grown for 15 days in culture was used. ALP (alkaline phosphatase) expression (an osteoblastic marker) was also found to be enhanced after 15 days. At both time points, adherent and non-adherent cells were reseeded, and RNA extractions were prepared 5 and 15 days later. A stronger RT-PCR product was observed with RNA extracted from originally adherent cells, suggesting the existence of less mature progenitors in the non-adherent population of bone marrow cells. The RT-PCR product of a non-osteoblastic marker gene (GAP-DH) was used as a control to compare RNA levels between samples.

In conclusion, bone marrow progenitor cells do not express OCP, but differentiate to more committed cells that do express this gene.

### EXAMPLE 14

### OCP Induction During Mesenchymal Cell Differentiation Towards Osteogenesis

Mesenchymal stem cells (MSC) are multipotent, self-renewing cell populations which undergo differentiation and commitment to give rise to monopotent cells of specified lineages, such as osteoblasts. The mechanisms of commitment and self-renewal are not fully understood, but may be regulated by factors such as Bone Morphogenetic Proteins (BMPs), differentiation factors such as retinoic acid (RA) and steroid hormones such as glucocorticoids. Furthermore, BMP and RA act synergistically to stimulate osteoblastic commitment and cell proliferation.

In order to find out if OCP expression is induced upon osteoblastic commitment, quiescent C3H10T1/2 murine MSC cultures were stimulated with BMP and RA for 24 hours and cultured in full medium for further 3 days. RNA was extracted from non-treated cells as well as from cells harvested at 24 hrs, 48 hrs and 72 hrs after the beginning of the treatment, and used for RT-PCR analysis with OCP-specific primers. In parallel, cells were stained for ALP to determine osteoblastic commitment. While ALP staining was apparent only on day 3 (72 hrs), OCP expression was augmented by day 1 (24 hrs), being undetectable in non-treated cultures. Further experiments have shown that even stronger ALP staining and OCP expression were observed on day 6 following proliferation and further differentiation of osteoprogenitor cells (FIG. 26).

These results demonstrate that upon osteoblastic commitment of MSCs, OCP expression is “switched-on” before the commencement of ALP expression, suggesting that this candidate is the earliest marker gene of osteoprogeniter cells found to date. It should be noted that our previous in situ hybridization results also demonstrated the presence of OCP transcripts only in very early chondro-/osteo-progenitor cells. These cells did not express ALP, and more mature ALP positive cells (in the trabecular bone) were OCP negative.

### EXAMPLE 15

### OCP Induction During Differentiation Switch of Pre-Myoblasts To Osteoblasts

Pre-myoblastic cells (C2C12) give rise to mature myoblasts. As with C3H10T1/2, the administration of BMP and RA to these cells can induce osteoblastic differentiation. To investigate the expression pattern of OCP during this differential switch we introduced BMP and RA to C2C12 cells and analyzed cell fate and expression pattern as above (for C3H10T1/2 cells). As expected OCP and ALP expression were induced 24 hrs post-BMP introduction (FIG. 26).

These assays once more demonstrate the involvement of OCP in the early stages of osteogenesis.

### EXAMPLE 16

### OCP Role in Osteogenesis

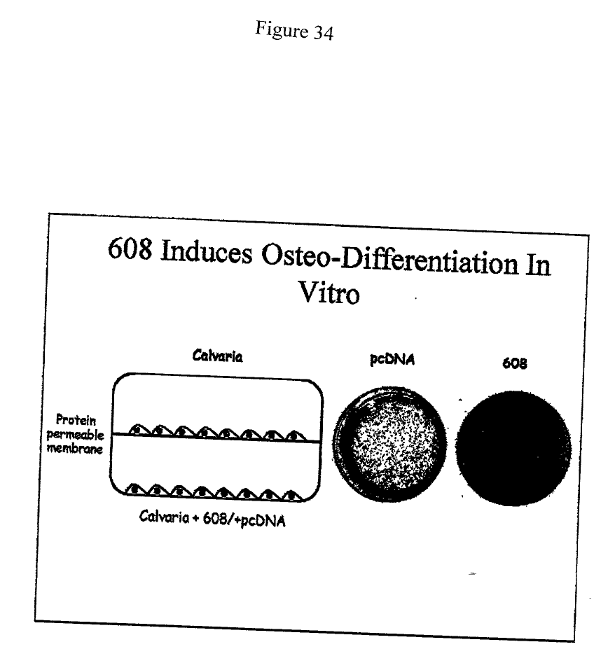

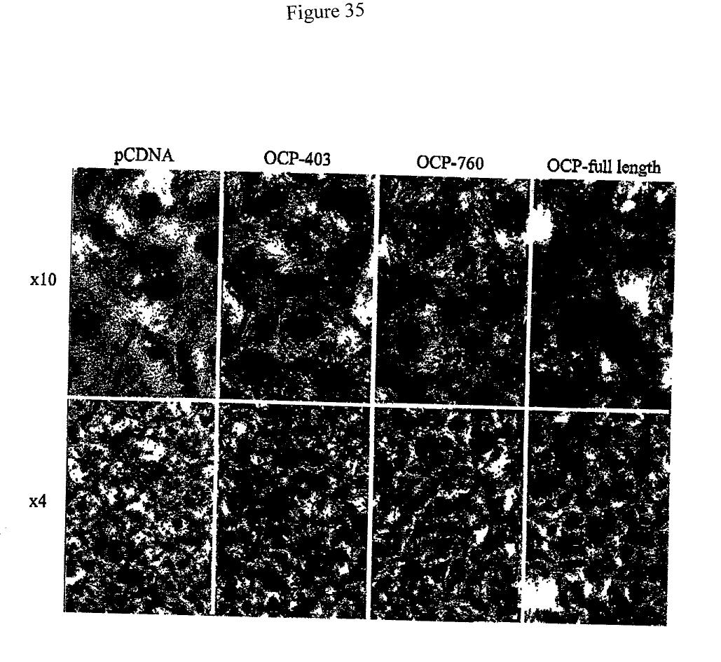

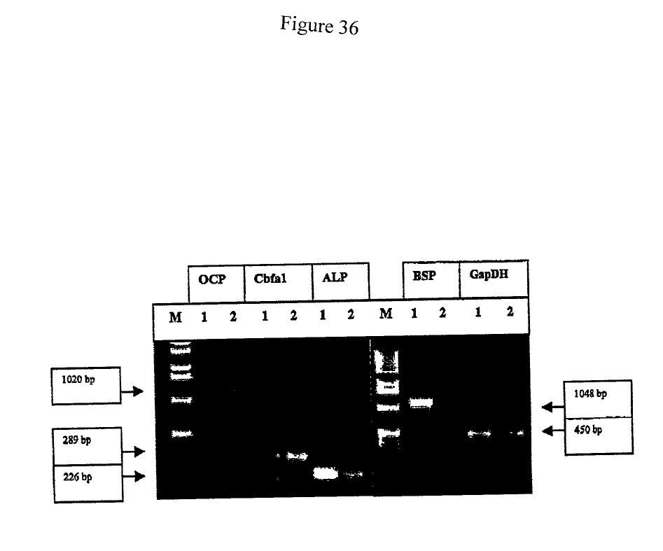

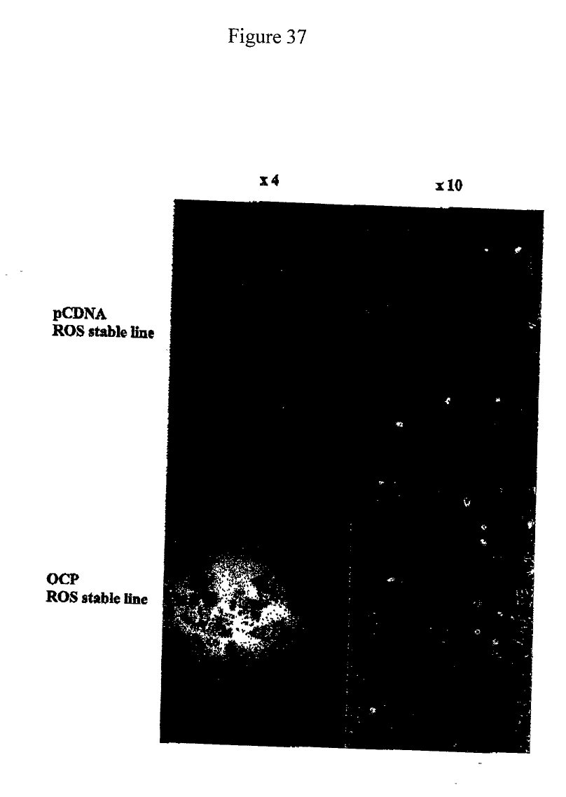

The ultimate test for the role of OCP as a crucial factor that induces osteoblast-related genes is its ability to up-regulate these genes in pre-osteoblastic and osteoblastic cells. In primary calvaria cells, transient transfection with a CMV promoter-driven OCP construct significantly up-regulated the expression of the osteogenic lineage marker ALP (FIG. 34 illustrates the induction in ALP staining). Transient transfections of two smaller deletion constructs of the OCP gene also gave the same induction (FIG. 35), suggesting that the N′ Terminal 403 amino acid protein stretch (which holds a signal peptide) is necessary and sufficient to augment osteoblastic proliferation and differentiation. In addition, stable transfection of OCP to ROS 17/2.8 (differentiating osteoblast cell line) cells, also substantially upregulated ALP & BSP expression, while repressing Cbfal transcription (Cbfal is known to be expressed early in the osteoblast lineage and to be transcriptionally downregulated during cellular aging of osteoblasts) (FIG. 36). In addition, marked increase in osteoblastic proliferation was observed (FIG. 37).

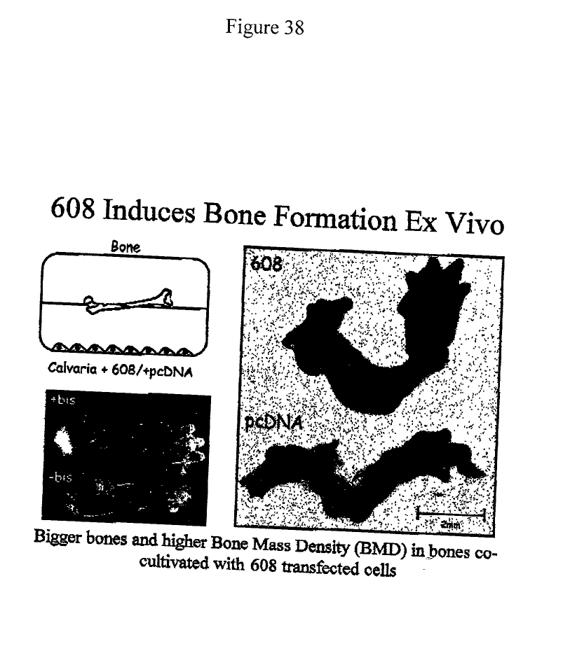

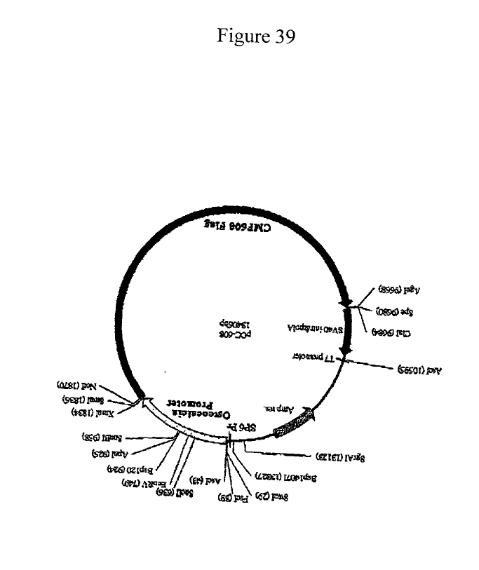

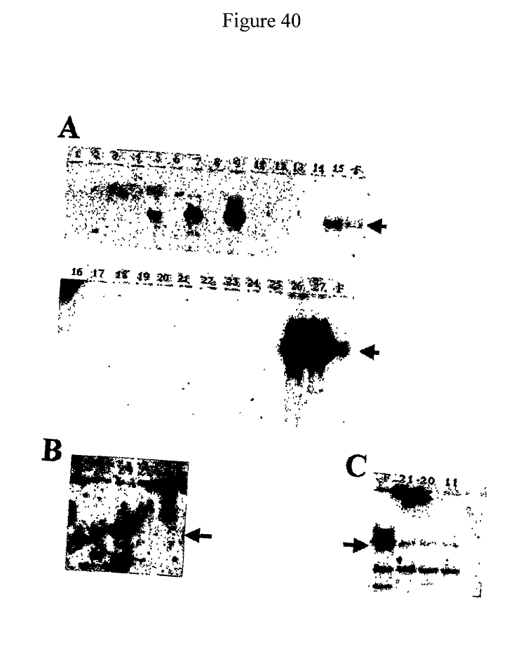

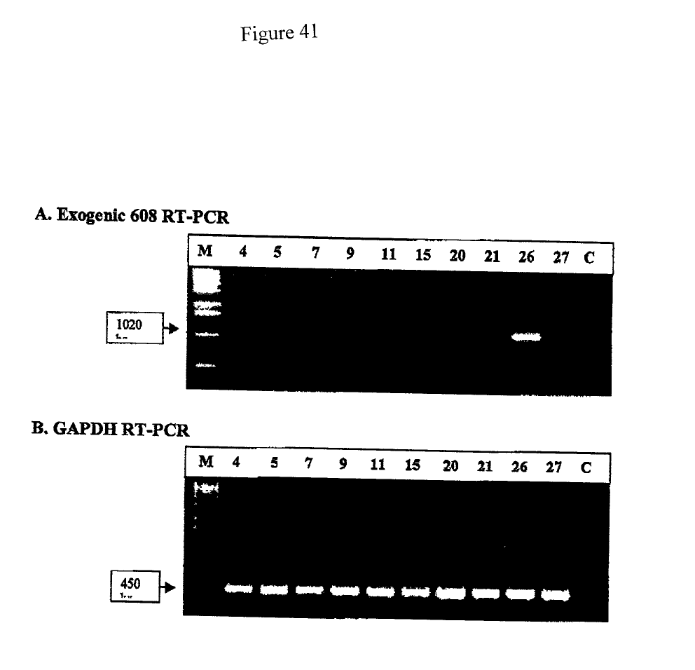

Further experiments have shown that the osteogenic effect of OCP expression in calvaria cells is non-cellautonomous. In a co-cultivation assay where OCP-transfected calvaria cells were cultured in the presence of nontransfected calvaria cells (that were grown on a millipore filter), the osteogenic induction effect was also evident as was illustrated in FIG. 38. The non-transfected cells that were cultured in the presence of OCP-transfected cells retained elevated ALP activity compared to control assays. No similar effects were observed upon transfection to the pluripotent progenitor C3H10T1/2 cells that can differentiate to myoblasts, osteoblasts, adipocytes or chondrocytes or to C2C12 pre-myoblast cells.

These results provide compelling evidence that OCP is an essential factor required for the initiation of the signaling cascade that leads to sequential expression of other phenotype-specific genes committed to the osteogenic lineage. In addition, these results support accumulation of an OCP-dependent osteogenesis factor that seems to act as a secreted factor. We have no data yet as to whether this factor is the OCP product or an OCP-induced factor.

### EXAMPLE 17

### Bone Culture Assays

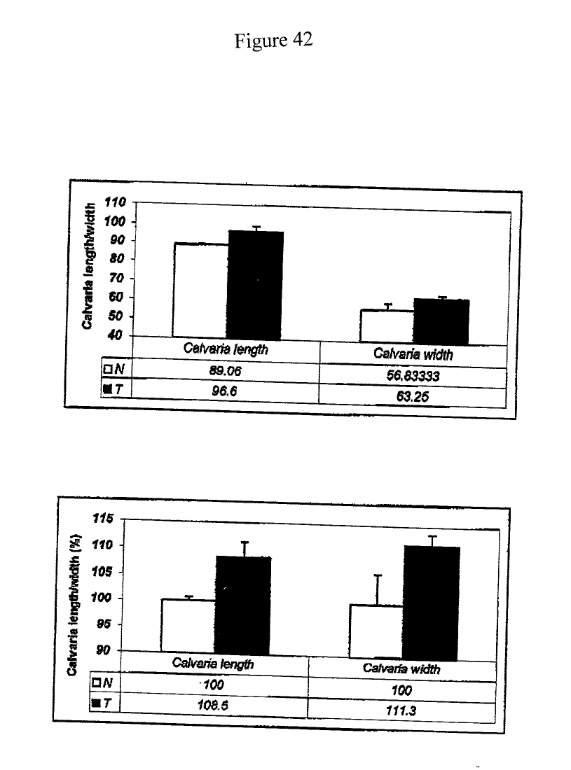

To further confirm the involvement of OCP in bone formation, we performed organ culture of E16 mouse embryonal limbs. The limb bones were stained with Alizarin Red following 6 days of culture to compare bone calcification rate. When the E16 mouse embryonal limbs were cocultivated with OCP-transfected calvaria cells, both endochondral and membranous ossification were enhanced as illustrated in FIG. 42. In contrast to the control limbs (cocultivated with vector-transfected calvaria cells), the OCP transfection to calvaria cells resulted in the formation of bones that are longer and wider in their proximal and distal extremities. Thus, we have shown that the osteogenic inducing effect of OCP that was observed in vitro, can be also demonstrated ex vivo by the induction of bone formation in cartilage bone rudiments. The role of OCP in bone rudiments probably mimics its role in endochondral ossification and bone development of mouse fetuses.

### EXAMPLE 18

### Oc-OCP Transgenic Mice

To verify the results presented in the present invention, the inventors generated transgenic mice in which 608 expression is induced in mature osteoblasts by coupling the OCP cDNA to the osteocalcin promoter.

Construction of the pOC-608 vector.

The Osteocalcin promoter was amplified using primers according to the literature. The promoter was taken from plasmid pSROCAT (Lian, J. et al. (1989) Structure of the rat osteocalcin gene and regulation of vitamin D-dependent expression. Proc. Natl. Acad. Sci. U.S.A. 86, 1143-1147) using SmaI and HindIII (blunted) and sub-cloned into the blunted BamHI and XbaI sites of the vector pMCS-SV producing the vector pOC-NSV.

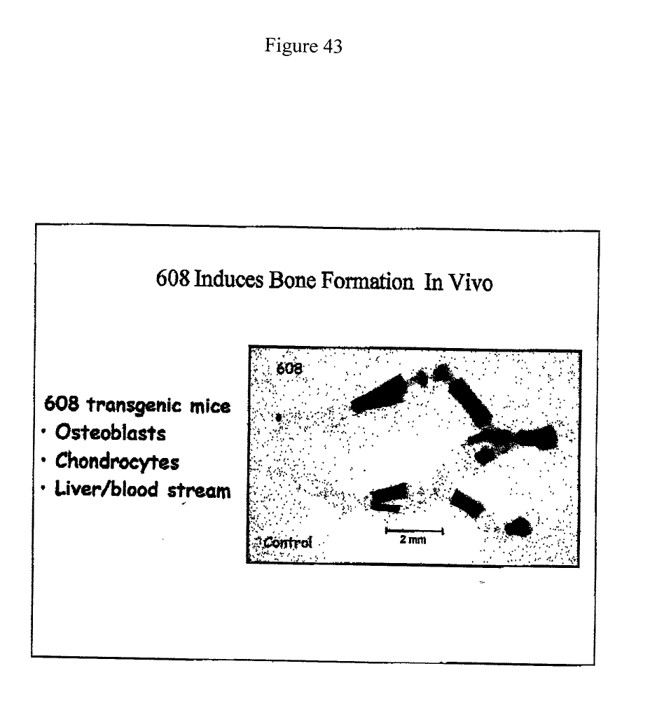

The CMF608 Flag fragment was isolated from the pCDA3.1-608 construct (FIG. 2) after NotI and SpeI digest. The fragment was sub-cloned into the NotI-SpeI sites of the pOC-MCS vector. The construct was verified by extensive sequencing (FIG. 43).

Preparation of DNA for microinjection

For the preparation of the DNA insert for microinjection the plasmid was digested with AscI (cuts at bp 43 and bp 10595). The ˜10.6Kb fragment was isolated from agarose gel using the Qiaex II kit (Qiagen cat No. 20021) and then purified over an Elutip-D column (Schleicher & Schuell cat. No. NA010/1).

Derivation of transgenic mice

The DNA was dissolved in a pure Tris/EDTA microinjection solution and adjusted to a concentration of 2 ng/μl. Standard pronuclear microinjection into fertilized eggs from the FVB/N strain and embryo transfer into ICR foster mothers was performed as described in the literature (see Manipulating the mouse embryo by Hogan, Beddington, Constantini & Lacy Cold Spring Harbor Laboratory Press).

Recovery of embryos

Foster mothers were sacrificed by cervical dislocation at day 18 post-embryo transfer. Embryos were recovered and placentas were taken for DNA preparation and analysis of the presence of the injected OC608-Flag DNA in the mouse genome.

Analysis of genomic DNA

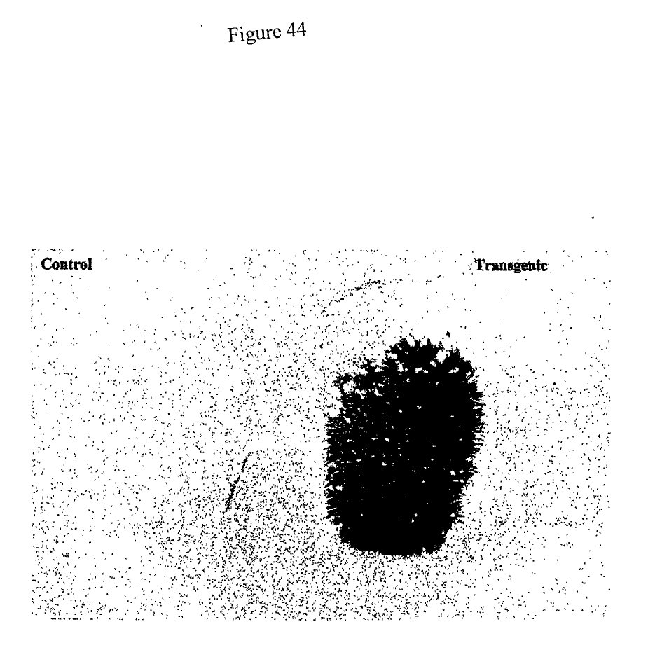

Mouse genomic DNA was recovered from the placenta using standard procedures (Laired P W et al Simplified mammalian DNA isolation procedure. Nucleic acid Research 19: 4293 (1991)). Genomic DNA was digested with EcoRV, separated on 1% agarose gel and blotted onto Nytran nylon membranes (Schleicher & Schuell). The blots were hybridized with a SV40 intr&polA labeled probe (see map) overnight and washed the following day. Membranes were exposed to X-ray films and developed after 24 and 48 hours (FIG. 44).

Analysis of OCP exogenic RNA expression

To determine which of the transgenic embryos expresses the exogenic OCP, total RNA was isolated from the hind legs as described (EZ-RNA, total RNA isolation kit, Biological industries). 5 μg of total RNA was assayed by RT-PCR as described (GIBCOBRL-SuperScript™ II). As a negative control, RT was omitted. PCR was performed for 30 cycles (1 min at 94° C., 1 min at 59° C.,and 2 min at 72° C.), using Taq polymerase (Promega) and either exogenic OCP or GapDH primers that amplify cDNA products of 1020 bp and 450 bp, respectively. The following primers were used for the detection of exogenic OCP:

Forward: 5′- GCACTGAACTGCTCTGTGGAT- 3′ (SEQ ID NO:21) &

Reverse

5′- CCACAGAAGTAAGGTTCCTTCAC - 3′ (SEQ ID NO:22)

Reaction products (5 μl per lane) were electrophoresed in 1.5% agarose and stained in ethidium bromide. As illustrated in FIG. 45, similar amounts of GapDH transcripts were detected in all RNA samples from all the embryos that were tested, indicating that differences in OCP transcript abundance did not reflect variation in the efficiency of the RT reaction. In addition, no GapDH PCR products were detected in any RNA samples when RT was omitted (data not shown). The results show that OCP was expressed by osteoblasts under the osteocalcin promoter transcriptional regulation only in embryos nos. 5, 7, 9, 11, 15, 21, 26 & 27 (FIG. 45).

Characterization of bone growth in osteocalcin promoter - 608 transgenic embryos

The results that are illustrated in FIGS. 46-48, suggest that over-expression of OCP during mice embryonal development (E17) results in increased endochondral (longitudinal) and membranous ossification of long bones and increased membranous ossification of calvaria flat bones. By summarizing all the above results we can conclude that this phenotype is caused primarily because of a profound increase in osteoblastic proliferation, differentiation and finally osteoblast activity. Further histological analysis of embryonic and adult transgenic mice is in process.

### EXAMPLE 19

### Creation of A Readout System

A readout system is created to identify small molecules that can either activate or inactivate 608 bone-precursor-specific promoter.

### EXAMPLE 20

### Bioinformatic Analysis of Human 608

Human genomic piece: AC024886.

Notice this is a newly submitted sequence from Sep. 6 2000. The sequence is found in htgs database but not in nt. There is no other genomic DNA corresponding to the rat cDNA. Alignment of this genomic piece against the rat cDNA using BLAST shows two areas of long alignments (and lots of smaller pieces):

| | |
|---|---|
| 1. | cDNA: 6462-8186 |
| | Genomic: 89228-90952 |
| | plus/plus orientation: 81% identity |
| 2. | cDNA: 5581-6451 |
| | Genomic: 107710-106840 |
| | plus/minus orientation: 80% identity |

Thus the genomic DNA was wrongly assembled in the region upstream
of position 6462 (according to the rat cDNA) it is flipped.
The information that was found in the Genbank report is as follows:
LOCUS AC024886 175319 bp DNA
HTG 06-SEP-2000
DEFINITION Homo sapiens chromosome 3 clone RP11-25K24,
WORKING DRAFT
SEQUENCE, 9 unordered pieces.
ACCESSION AC024886
VERSION AC024886.10 GI:9438330
KEYWORDS HTG; HTGS_PHASE1; HTGS_DRAFT.
SOURCE human.
*NOTE: This is a 'working draft' sequence. It currently consists of 9
contigs. The true order of the pieces is not known and their order in this
sequence record is arbitrary. Gaps between the contigs are represented as
runs of N, but the exact sizes of the gaps are unknown. This record will be
updated with the finished sequence as soon as it is available and the
accession number will be preserved.

| | | |
|---|---|---|
| * | 1 | 62523: contig of 62523 bp in length |
| * | 62524 | 62623: gap of unknown length |
| * | 62624 | 85445: contig of 22822 bp in length |
| * | 85446 | 85545: gap of unknown length |
| * | 85546 | 106059: contig of 20514 bp in length |
| * | 106060 | 106159: gap of unknown length |
| * | 106160 | 127908: contig of 21749 bp in length |
| * | 127909 | 128008: gap of unknown Length |
| * | 128009 | 143068: contig of 15060 bp in length |
| * | 143069 | 143168: gap of unknown length |
| * | 143169 | 158734: contig of 15566 bp in length |
| * | 158735 | 158834: gap of unknown length |
| * | 158835 | 170042: contig of 11208 bp in length |
| * | 170043 | 170142: gap of unknown length |
| * | 170143 | 173715: contig of 3573 bp in length |
| * | 173716 | 173815: gap of unknown length |
| * | 173816 | 175319: contig of 1504 bp in length. |

### a. Mapping the Human Genomic 608 Exons

Ten exons were mapped on the rat cDNA sequence from base 107 to 6451. This means that we probably lack the first exon on the human genomic piece. The human genomic piece used from the public genomic entry (AC024886) upstream (19090 bases) of base 6462 of cDNA (reverse complement from base of AC024886 92001 to 111090) was run along with the rat cDNA using the program ExonMapper of Genomatix. In table xxx base 1 is actually 1131 in the genomic piece used so that the actual genomic location starts at 91870.

Two additional exons were mapped on the rat cDNA sequence from base 6462 to 8883. This means that we lack bases 6452-6461. The human genomic piece used is from base 165,337 to 175667 (10, 341 bases). Same type of program was run on the QBI genomic mouse 608.

1. Connecting the exons/introns borders from the genomic sequence yielded the predicted human and mouse cDNAs. The mouse and human predicted cDNAs were modified in order to allow frame shifts that will produce a good multiple alignment of the human, mouse and rat proteins. Alignment was done using CLUSTALX and Pretty.

The modifications of the cDNA after the alignment of human cDNA to rat protein by Gene Wise were as follows:

| Position | Change |
|---|---|
| 1111 | −g |
| 4154 | −c |
| 4538 | +g |
| 4730 | −a |
| 4744-5 | −aa |
| 4830 | +c |
| 4852 | −g |
| 4902 | +t |
| 4942 | +c |
| 5370 | +t |
| 5387 | −a |
| 5395 | +c |

The corrections of frame-shifts in the mouse sequence of 608 were as follows:

| Position | Change |
|---|---|
| 678 | −c |
| 1106 | −a |

### Changes Glossary

−x deletion of nucleotide x in the cDNA sequence

+x insertion of nucleotide x in the cDNA sequence

NOTE: all changes positions are in relation to the original sequence

Chromosomal Location on the human chromosome:
Two different types of data exist.
a. Genomic piece AC024886 has identity to the following fragment:
LOCUS HUMHHIRE 5856 bp DNA PRI 23-JUN-1999
DEFINITION Human gene for histamine H1-receptor, complete cds.
ACCESSION D14436
VERSION D14436.1 GI:506335
KEYWORDS G-protein associated; histamine H1 receptor.
SOURCE Homo sapiens leukocyte DNA.
ORGANISM Homo sapiens

Eukaryota; Metazoa; Chordata; Craniata; Vertebrata; Mammalia;
Eutheria; Primates; Catarrhini; Hominidae; Homo.

REFERENCE 1 (bases 1 to 5856)
AUTHORS Fukui, H., Fujimoto, K., Mizuguchi, H., Sakamoto, K.,

Horio, Y., Takai, S., Yamada, K. and Ito, S.

TITLE Molecular cloning of the human histamine H1 receptor gene
JOURNAL Biochem. Biophys. Res. Commun. 201 (2), 894-901 (1994)
MEDLINE 94271250
REFERENCE 2 (bases 1 to 5856)
AUTHORS Fukui, H.
TITLE Direct Submission
JOURNAL Submitted (10-FEB-1993) to the DDBJ/EMBL/GenBank
databases.

| | Hiroyuki Fukui, Osaka University Faculty of Medicine, Dept. | |
|---|---|---|
| | Pharmacol. II.; 2-2 Yamadaoka, Suita, Osaka 565, Japan | |
| | (E-mail:a62520a@center.osaka-u.ac.jp, Tel: 06-875-7366, | |
| | Fax: 06-875-7368) | |
| FEATURES | | Location/Qualifiers |
| | source | 1..5856 |

/organism=“Homo sapiens”
/db_xref=“taxon:9606”
/cell_type=“leukocyte”
5'UTR 1..2164

| | |
|---|---|
| CDS | 2165..3628 |

Alignment information:

Identities = 315/335 (94%),
hrhl : 4 −338
AC024886: 41662 - 41328

This still raises questions whether hrhl is located on this genomic piece.
Hrhl is mapped to chromosome 3 (like our genomic piece) and to 3p25!
b. Identity to STS at 3q:
>gb|G54348.1|G54348 25A23sp6 Human BAC clone Homo sapiens STS
genomic, sequence tagged site

Length = 433

Score = 646 bits (326), Expect = 0.0
Identities = 397/414 (95%), Gaps = 6/414 (1%)
Strand = Plus/Plus
AC024886: 30130-30538
STS: 20-432
More information on the STS:
LOCUS G54370 616 bp DNA STS 16-MAR-2000
DEFINITION 564F23t7 Human BAC clone Homo sapiens STS genomic,
sequence tagged site.
ACCESSION G54370
VERSION G54370.1 GI:7259639
KEYWORDS STS.
SOURCE human.
ORGANISM Homo sapiens

Eukaryota; Metazoa; Chordata; Craniata; Vertebrata; Euteleostomi;
Mammalia; Eutheria; Primates; Catarrhini; Hominidae; Homo.

REFERENCE 1 (bases 1 to 616)
AUTHORS Joensuu, T., Hamalainen, R., Lehesjoki, A. E.,
de la Chapelle, A. and Sankila, E. M.
TITLE A sequence-ready map of the Usher syndrome type III
critical region on chromosome 3q
JOURNAL Genomics 63 (3), 409-416 (2000)
An additional simillar STS:
>gb|G54348.1|G54348 25A23sp6 Human BAC clone Homo sapiens STS
genomic, sequence tagged site
Length = 433
Score = 646 bits (326), Expect = 0.0
Identities = 397/414 (95%), Gaps = 6/414 (1%)
Strand = Plus/Plus
AC024886: 30130-30538
STS: 20-432

### EXAMPLE 21

### Preparation of Polyclonal Antibodies

Polyclonal antibodies covering the whole 608 putative protein are prepared for the identification of the active form of this protein by methods well-known in the art (the structure of 608 resembles that of growth factor precursors). Polyclonal antibodies are identified and the recombinant active form of 608 is prepared. The activities of the polyclonal antibodies are tested in vivo in mice. The active fragment of the 608 protein is likely to constitute a fraction of the 608 protein.

### EXAMPLE 22

### Stretch of Basic Amino Acids Found at the Boundary of The Rat and Human 608 Proteins, and its Implications

We have noted that the homology between the rat and human N-terminal portions of the 608 protein is especially significant within the first 250 amino acids.

At the boundary of this conserved region, we have noticed a completely conserved stretch of basic amino acids: KCKKDR (aa 242-247 and 240-245, in rat and human proteins, respectively). The stretches of basic amino acids frequently serve as protease cleavage sites. The fact that such a stretch is found on the boundary of more or less conserved sequences and the fact that it occurs within the C-terminal LRR, a generally conserved domain, may suggest an underlying biological significance.

Accordingly, we hypothesized that the 608 protein may undergo post-translational processing through the cleavage of its highly conserved N-terminal portion and that this portion is an active part of the 608 protein or possesses at least part of its biological activities. Since the resulting˜25 kD protein preserves the signal peptide, it is supposed to be secreted out of cells regardless of whether the cleavage itself occurs inside the cell or outside of it.

To test whether the hypothetical 25 kD cleavage product of the 608 protein is responsible for the observed osteogenic activity of medium conditioned by 608-transfected calvaria cells, we constructed a pCDNA vector that contained the N-terminal portion of rat 608 cDNA coding for amino acids 1- 241 (not including the KCKKDR stretch) and transiently expressed it in rat calvaria cells. The transfected cells were assayed for their ability to induce bone formation both in co-cultured non-transfected calvaria cells and in ex vivo cultured E16 mouse embryo (as described above for the full-length 608 cDNA). The results clearly indicated that the secreted N-terminal portion of 608 protein was sufficient to stimulate osteogenesis in co-cultured cells and embryo bones. The biologically active 25 kD N-terminal cleavage product of 608 can be used for treatment and/or prevention of osteoporosis, fracture healing, bone elongation and periodontosis. As an indirect product (inhibition by either chemicals or by neutralizing monoclonal antibodies) can be used for treatment and/or prevention of osteoarthritis, osteopetrosis, and osteosclerosis

Having thus described in detail preferred embodiments of the present invention, it is to be understood that the invention defined by the appended claims is not to be limited by particular details set forth in the above description as many apparent variations thereof are possible without departing from the spirit or scope thereof.

### REFERENCES

Reddi, A. H. (1981) Collagen Rel. Res., 1:209-226;

Melton, L. J. III. (1995) Bone Miner. Res., Vol. 10, page 175.

Consensus Development Conference: Prophylaxis and Treatment of Osteoporosis: Am. J. Med. 94:646-650, 1993;

Parfitt A M: Quantum concept of bone remodeling and turnover: Implications for the pathogenesis of osteoporosis. Calcif Tissue Int 28:1-5, 1979

Parfitt A M: Bone remodeling in the pathogenesis of osteoporosis. Res Staff Physician, December:60-72, 1981

Parfitt A M: Bone remodeling: Relationship to the amount and structure of bone, and the pathogenesis and prevention of fractures. In Osteoporosis: Etiology, Diagnosis, and Management (Riggs B L, Melton L J, eds.). Raven Press, New York, 1988, pp 74-93

Garnero et al.: Genetic influence on bone turnover in postmenopausal twins. J Clin Endocrinol Metab. 81(1):140-46, 1996.

Johnston et al.: Pathogenesis of osteoporosis. Bone 17(2 Suppl): 19S-22S, 1995.

Rosen, C J: Beyond one gene-one disease: Alternative strategies for deciphering genetic determinants of osteoporosis. Calcif Tissue Int 60(3):225-28, 1997.

Keen et al.: Genetic factors in osteoporosis. What are the implications for prevention and treatment? Drugs Aging (New Zealand) 11(5):333-7, 1997.

Jouanny et al.: Environmental and genetic factors affecting bone mass. Similarity of bone density among members of healthy families. Arthritis Rheum 38(1):61-67, 1995.

Gong et al.: Osteoporosis-pseudoglioma syndrome, a disorder affecting skeletal strength and vision, is assigned to chromosome region 11q12-13. Am J Hum Genet 59(1):146-51, 1996.

Cummings: Treatable and untreatable risk factors for hip fracture. Bone 18(3 Suppl):165S-167S, 1996.

Wasnich: Vertebral fracture epidemiology. Bone 18(3 Suppl):179S-183S, 1996.

Wark: Osteoporotic fractures: background and prevention strategies. Maturitas (Ireland) 23(2):193-207, 1996.

Livshits et al.: Population biology of human aging: segregation analysis of bone age characteristics. Hum Biol 68:540-54 1996.

Lonzer et al.: Effects of heredity, age, weight, puberty, activity and calcium intake on bone mineral density in children. Clin Pediatr 35(4):185-9, 1996.

Gilboa, E, Eglitis, M A, Kantoff, P W, Anderson, W F: Transfer and expression of cloned genes using retroviral vectors. BioTechniques 4(6):SO4-512, 1986.

Culver, 1998. Site-Directed recombination for repair of mutations in the human ADA gene. (Abstract) Antisense DNA & RNA based therapeutics, February, 1998, Coronado, Calif.

Huston et al, 1991 “Protein engineering of single-chain Fv analogs and fusion proteins” in Methods in Enzymology (ii Langone, ed.; Academic Press, New York, N.Y.) 203:46-88.

Johnson and Bird, 1991 “Construction of single-chain Fvb derivatives of monoclonal antibodies and their production in Escherichia coli in Methods in Enzymology (J J Langone, ed.; Academic Press, New York, N.Y.) 203:88-99.

Mernaugh and Mernaugh, 1995 “An overview of phage-displayed recombinant antibodies” in Molecular Methods In Plant Pathology (RP Singh and US Singh, eds.; CRC Press Inc., Boca Raton, Fla.) pp. 359-365. Cold Springs Harbor Laboratory Press, Cold Spring Harbor, N.Y., 1989

What is claimed is:

1. An isolated nucleic acid molecule comprising nucleotides having a sequence set forth in SEQ ID NO:1, SEQ ID NO:3, SEQ ID NO:6 or SEQ ID NO:20, complements thereof and a polynucleotide having a sequence that differs from SEQ ID NO:1, SEQ ID NO:3, SEQ ID NO:6 or SEQ ID NO:20 due to the degeneracy of the genetic code or a functional portion thereof or a polynucleotide which is at least substantially homologous or identical thereto.

2. The isolated nucleic acid molecule of claim 1, wherein the nucleic acid molecule comprises a polynucleotide having at least 15 nucleotides from SEQ ID NO:1, SEQ ID NO:3, SEQ ID NO:6 or SEQ ID NO:20.

3. A composition comprising the isolated nucleic acid molecule of claim 1.

4. A vector comprising the isolated nucleic acid molecule of claim 1.

5. A composition comprising the vector of claim 4.

6. A method for preventing, treating or controlling osteoporosis, or for fracture healing, bone enlongation or osteopenia, periodontosis, or low bone density or or other conditions involving mechanical stress or a lack thereof in a subject, comprising administering to the subject an effective amount of a composition as claimed in claim 3.

7. A method for preventing, treating or controlling osteoporosis, or for fracture healing, bone enlongation or osteopenia, periodontosis, bone fractures or low bone density or other factors causing or contributing to osteoporosis or symptoms thereof or other conditions involving mechanical stress or a lack thereof in a subject, comprising administering the vector to the subject as claimed in claim 4.

8. A method for preparing a polypeptide comprising expressing the isolated nucleic ?

17. The isolated polypeptide of claim 15, wherein the the functional portion comprises a polypeptide having a molecular weight of about 25 kD.

18. A composition comprising one or more isolated polypeptides of claims 13.

19. An antibody elicited by a polypeptide of claim 13 or a functional portion thereof.

20. A composition comprising the antibody of claim 19 or a functional portion thereof.

21. A method for treating or preventing osteoporosis, or for fracture healing, bone enlongation, or periodontosis in a subject, comprising administering to the subject an effective amount of the isolated polypeptide of claim 16.

22. A method of treating or preventing osteoarthritis, osteopetrosis, or osteosclerosis, comprising administering to a subject an effective amount of a chemical or a neutralizing monoclonal antibodies which inhibit the activity of the polypeptide of claim 16.
